(19) United States
(12) Patent Application Publication
(10) Pub. No.: US 2011/0190511 A1
(43) Pub. Date: Aug. 4, 2011

(54) Methods For Preparing Fluoroalkyl Arylsulfinyl Compounds And Fluorinated Compounds Thereto

(75) Inventors: Teruo Umemoto, Westminster, CO (US); Rajendra P. Singh, Broomfield, CO (US)

(73) Assignee: IM&T RESEARCH, INC., Denver, CO (US)

(21) Appl. No.: 12/663,605

(22) PCT Filed: Aug. 17, 2009
(86) PCT No.: PCT/US09/54067

§ 371 Date: Jan. 14, 2010

Related U.S. Application Data
(60) Provisional application No. 61/089,801, filed on Aug. 18, 2008.
(60) Provisional application No. 61/152,439, filed on Feb. 13, 2009.
(60) Provisional application No. 61/155,410, filed on Feb. 25, 2009.

(51) Int. Cl. C07D 207/48; C07C 313/04; C07C 313/06

(52) U.S. Cl. 548/531; 558/61; 564/101; 548/542

(57) ABSTRACT

Novel preparative methods for fluoroalkyl arylsulfinyl compounds are disclosed. Fluorinated compounds as useful fluorinated compounds, intermediates, or builing blocks are disclosed. Useful applications of the fluoroalkyl arylsulfinyl compounds are shown.

### TECHNICAL FIELD

The invention relates to preparative method for fluoroalkyl arylsulfinyl compounds and to useful fluorinated compounds thereto.

### BACKGROUND OF THE INVENTION

Fluorine-containing compounds have found wide use in medical, agricultural, and electronic materials as well as in other like industries [see Chemical & Engineering News, June 5, pp. 15-32 (2006); Angew. Chem. Ind. Ed., Vol. 39, pp. 4216-4235 (2000)]. These compounds show specific biologic activity or physical properties based on the presence of one or more fluorine atoms. A particular drawback in their usefulness is the scarcity of natural fluorine-containing compounds, requiring most such compounds to be prepared through organic synthesis. Therefore, there has been extensive study and research on synthetic methodologies for the preparation of fluorine-containing compounds [see, for example, Chem, Rev., Vol. 108, pp. PR1-PR43 (2008); Chem. Rev., Vol. 105, pp. 827-856 (2005); Tetrahedron, Vol. 52, pp. 8619-8683 (1996); Chem. Rev., Vol. 92, pp. 505-519 (1992)].

Examples of widely known methods of preparation of fluorine-containing compounds include a method of direct fluorination with fluorine gas (F₂); halogen exchange reaction by action of hydrogen fluoride (HF) or an alkali metal salt of fluorine such as KF; Schiemann reaction in which an aryldiazonium tetrafluoroborate derived from an arylamine is tranformed to an aryl fluoride; a method using a mixture of HF and a base such as pyridine or triethylamine; a method using an interhalogen fluoride or a hypervalent fluoride such as ClF, BrF, IF, XeF₂, BrF₃, IF₅, and ArIF₂; a method using a specific nucleophilic fluorinating agent such as SF₄, DAST, DeoxoFluor™ reagent; FAR (fluoroalkylamino reagent) such as Yarovenko-Raksha reagent, Ishikawa reagent, 2,2-difluoro-1,3-dimethylimidazolidine, N,N-diethyl-α,α-difluoro-(m-methylbenzyl)amine, and N,N-dimethyl-1,1,2-trifluoroethylamine; a method using a specific electrophilic fluorinating agent such as N-fluoropyridinium salts, N-chloromethyl-N-fluorodiazoniabicyclo[2.2.2]octane salt, and N-fluorobis(phenylsulfonyl)amide; and a method of electrolytic fluorination [see, for example, Chem. Rev., Vol. 92, pp. 505-519 (1992); Chem. Rev., Vol. 86, pp. 997-1018 (1986); Chemistry and Industry, Vol. 21, pp. 56-63 (January 1978); Israel Journal of Chemistry, Vol. 17, pp. 71-79 (1978); Organic Reactions, Vol. 5, pp. 193-228 (1968); J. Fluorine Chem., Vol. 109, pp. 25-31 (2001); Chem. Rev., Vol. 96, pp. 1737-1755 (1996)].

However, conventional methods that utilize F₂, HF, SF₄, and DAST have safety problems because of their highly toxic, corrosive, or explosive nature. In addition, Deoxo-Fluor™ reagent has low thermal stability. The method using a mixture of HF and a base usually requires a large excess of the mixture to obtain a high yield. The FAR reagents have low reactivity, so the scope of their application is narrow. Methods using an electrophilic fluorinating agent are limited to the fluorination of a nucleophilic substrate such as a carbanion, an electron-rich aromatic compound, and a trimethylsilyl enol ether. The electrolytic fluorination is also limited to a specific substrate because selectivity is low in the fluorination.

Examples of methods of fluorinating a diol or an amino alcohol include fluorination with DAST, Deoxo-Fluor™ reagent, N,N-diethyl-α,α-difluoro-(m-methylbenzyl)amine, or a cyclic acetal of N,N-diethyl-4-methoxybenzamide [J. Org. Chem., Vol. 40, pp. 574-578 (1975); J. Fluorine Chem., Vol. 125, pp. 1869-1872 (2004); Chem. Commun., 2005, pp. 3589-3590; Synlett, 2006 (11), pp. 1744-1746; J. Fluorine Chem., Vol. 128, pp. 1121-1125 (2007)]. The fluorination of a diol or an amino alcohol with DAST or Deoxo-Fluor™ reagent produces a corresponding or rearranged difluoro compound. The fluorination of a diol or an amino alcohol with N,N-diethyl-α,α-difluoro-(m-methylbenzyl)amine or a cyclic acetal of N,N-diethyl-4-methoxybenzamide produces a fluoroalkyl arylcarboxylate or a fluoroalkyl arylamide. The fluoroalkyl arylcarboxylate or arylamide is derived to a fluoro alcohol or a fluoro amine.

However, DAST and Deoxo-Fluor™ reagent have the same problems as described above. N,N-Diethyl-α,α-difluoro-(m-methylbenzyl)amine and a cyclic acetal of N,N-diethyl-4-methoxybenzamide require microwave irradiation or high reaction temperature because of low reactivity.

From an alternative point of view, there are basically two methodologies to prepare a fluoro organic compound: (1) a fluorination methodology includes an available non-fluoro compound fluorinated with a fluorinating agent to produce a desired fluoro compound, or the fluorination is conducted at the final or almost final stage in the preparation processes to give a desired fluoro compound; and (2) a fluorinated building block (fluorinated synthon) methodology where a desired fluoro compound is constructed by multi-step preparation processes starting from a fluorinated building block (a fluorinated synthon), “a fluorinated molecule possessing a reactive site” [see, for example, Chemical & Engineering News, June 5, pp. 15-32 (2006): Fluorine-containing Synthons; ed by V. V. Soloshonok; ACS Symposium Series 911, American Chemical Society, Washington, D.C., 2005, pp. 119-134]. Thus, new developments of useful fluorination techniques, fluorinated intermediate compounds (fluorinated building blocks), and synthetic methods using the fluorinated intermediates are particularly important for synthesizing desired fluoro organic compounds which possess a fluorine atom(s) at a specific position in a stereo structure. The position and stereo chemistry of a fluorine atom in a molecule is very important for appropriate biologic and/or physical activity.

However, there are still many problems in conventional methodologies, for example, selectivity in fluorination is not sufficient, so regio and/or stereo isomeric fluoro compounds are produced, which are difficult to separate/isolate; also, there is a lack of safe, ease-to-handling, cost-effectiveness, and selectivity for preparing a desired fluoro compound; and many preparative steps are required to prepare a desired fluoro product due to the lack of suitable fluorinated intermediates or building blocks (fluorinated synthons), so total yield is low.

Therefore, problems with the production methods for fluoro organic compounds have made it difficult to prepare fluoro materials in which a fluorine atom is located in a correct position and stereo chemistry, especially in a safe, cost effective and timely fashion.

The present invention is directed toward overcoming one or more of the problems discussed above.

### SUMMARY OF THE INVENTION

The present invention provides a new and useful method for preparing a fluoroalkyl arylsulfinyl compound having a formula (I):

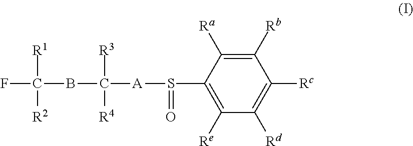

by reacting an oxygen-containing compound having a formula (II) with arylsulfur trifluoride having a formula (III):

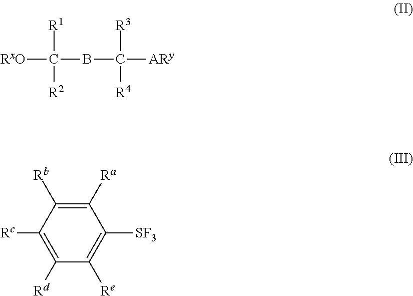

Embodiments of the present invention also include methods wherein the arylsulfur trifluoride of formula (III) is prepared by a method comprising reacting arylsulfur halotetrafluoride of formula (IV) with a reducing substance.

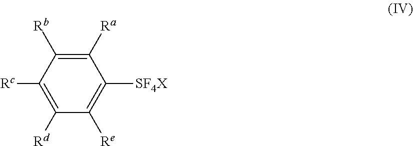

The present invention also provides a useful method(s) for preparing a fluoroalkyl arylsulfinyl compound having a formula (I) by reacting an oxygen-containing compound having a formula (II) with arylsulfur halotetrafluoride having a formula (IV) in the presence of a reducing substance.

The present invention also provides novel fluoroalkyl arylsulfinyl compounds of formulas (Ia), (Ib), (Ic), and (Id):

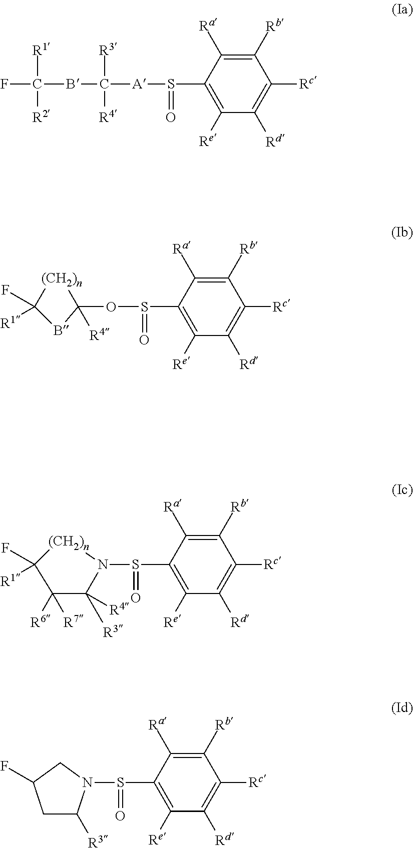

In addition, the present invention provides useful methods for preparing fluoroalkyl arylsulfinyl compounds of formulas (Ie) and (If).

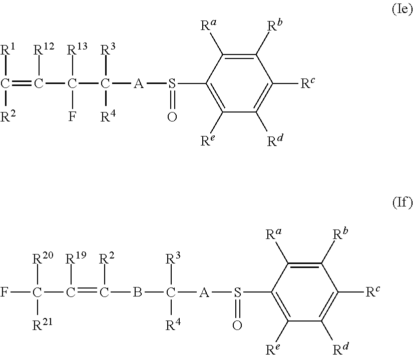

These and various other features as well as advantages which characterize embodiments of the invention will be apparent from a reading of the following detailed description and a review of the appended claims.

### DETAILED DESCRIPTION OF THE INVENTION

The invention provides novel processes for preparing fluoroalkyl arylsulfinyl compounds, as well as provides useful fluoroalkyl sulfinyl compounds thereto.

The present invention provides a method (Scheme 1, Process I) for preparing a fluoroalkyl arylsulfinyl compound having a formula (I), which comprises reacting an oxygen-containing compound having a formula (II) with an arylsulfur trifluoride having a formula (III):

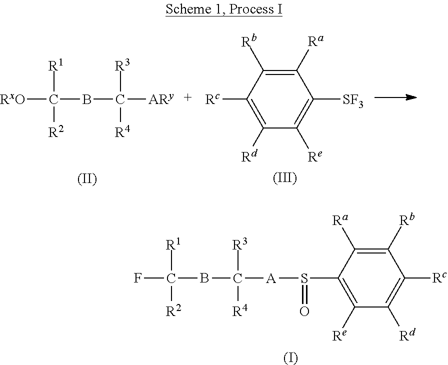

in which:

R¹, R², R³, and R⁴ each is independently a hydrogen atom, a substituted or unsubstituted alkyl group having 1 to 20 carbon atoms, preferably 1 to 14 carbon atoms, a substituted or unsubstituted alkenyl group having 2 to 20 carbon atoms, preferably 2 to 14 carbon atoms, a substituted or unsubstituted alkynyl group having 2 to 20 carbon atoms, preferably 2 to 14 carbon atoms, a substituted or unsubstituted aryl group having 6 to 20 carbon atoms, preferably 6 to 14 carbon atoms, a substituted or unsubstituted heterocyclyl group having 1 to 20 carbon atoms, a substituted or unsubstituted alkoxy group having 1 to 20 carbon atoms, preferably 1 to 14 carbon atoms, a substituted or unsubstituted acyl group having 1 to 20 carbon atoms, preferably 1 to 14 carbon atoms, a substituted or unsubstituted alkoxycarbonyl group having 2 to 20 carbon atoms, preferably 2 to 14 carbon atoms, a substituted or unsubstituted aryloxycarbonyl group having 7 to 20 carbon atoms, preferably 7 to 14 carbon atoms, a substituted or unsubstituted (heterocyclyl)oxycarbonyl group having 2 to 20 carbon atoms, preferably 2 to 14 carbon atoms, or a cyano group;
R^{a}, R^{b}, R^{e}, R^{d}, and R^{e} each is independently a hydrogen atom, a halogen atom, a substituted or unsubstituted alkyl group having 1 to 10 carbon atoms, preferably 1 to 4 carbon atoms, a nitro group, or a cyano group;
A is an oxygen atom or NR⁵ in which R⁵ is a hydrogen atom, a substituted or unsubstituted alkyl group having 1 to 20 carbon atoms, preferably 1 to 14 carbon atoms, a substituted or unsubstituted aryl group having 6 to 20 carbon atoms, preferably 6 to 14 carbon atoms, a substituted or unsubstituted heterocyclyl group having 1 to 20 carbon atoms, preferably 1 to 14 carbon atoms, a substituted or unsubstituted acyl group having 1 to 20 carbon atoms, preferably 1 to 14 carbon atoms, a substituted or unsubstituted alkoxycarbonyl group having 2 to 20 carbon atoms, a substituted or unsubstituted aryloxycarbonyl group having 7 to 20 carbon atoms, preferably 7 to 14 carbon atoms, a substituted or unsubstituted (heterocyclyl)oxycarbonyl group having 2 to 20 carbon atoms, preferably 2 to 14 carbon atoms, a substituted or unsubstituted alkylsulfonyl group having 1 to 20 carbon atoms, preferably 1 to 14 carbon atoms, a substituted or unsubstituted arylsulfonyl group having 6 to 20 carbon atoms, preferably 6 to 14 carbon atoms, a substituted or unsubstituted (heterocyclyl)sulfonyl group having 1 to 20 carbon atoms, preferably 1 to 14 carbon atoms, or a R¹⁴R¹⁵R¹⁶Si group in which R¹⁴, R¹⁵, and R¹⁶ each is independently an alkyl group having 1 to 10 carbon atoms, preferably 1 to 4 carbon atoms, an aralkyl group having 7 to 10 carbon atoms, or an aryl group having 6 to 10 carbon atoms;
B is

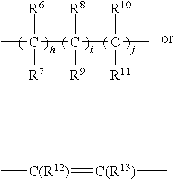

in which:
R⁶, R⁷, R⁸, R⁹, R¹⁰, R¹¹, R¹² and R¹³ each is independently a hydrogen atom, a halogen atom, a substituted or unsubstituted alkyl group having 1 to 20 carbon atoms, preferably 1 to 14 carbon atoms, a substituted or unsubstituted alkenyl group having 2 to 20 carbon atoms, preferably 2 to 14 carbon atoms, a substituted or unsubstituted alkynyl group having 2 to 20 carbon atoms, preferably 2 to 14 carbon atoms, a substituted or unsubstituted aryl group having 6 to 20 carbon atoms, preferably 6 to 14 carbon atoms, a substituted or unsubstituted heterocyclyl group having 1 to 20 carbon atoms, preferably 1 to 14 carbon atoms, a substituted or unsubstituted alkoxy group having 1 to 20 carbon atoms, preferably 1 to 14 carbon atoms, a substituted or unsubstituted aryloxy group having 6 to 20 carbon atoms, preferably 6 to 14 carbon atoms, a substituted or unsubstituted (heterocyclyl)oxy group having 1 to 20 carbon atoms, preferably 1 to 14 carbon atoms, a substituted or unsubstituted acyl group having 1 to 20 carbon atoms, preferably 1 to 14 carbon atoms, a substituted or unsubstituted acyloxy group having 1 to 20 carbon atoms, preferably 1 to 14 carbon atoms, a substituted or unsubstituted alkoxycarbonyl group having 2 to 20 carbon atoms, preferably 2 to 14 carbon atoms, a substituted or unsubstituted aryloxycarbonyl group having 7 to 20 carbon atoms, preferably 7 to 14 carbon atoms, a substituted or unsubstituted (heterocyclyl)oxycarbonyl group having 2 to 20 carbon atoms, preferably 2 to 14 carbon atoms, a substituted or unsubstituted amino group having 20 or less carbon atoms, preferably 14 or less carbon atoms, a substituted or unsubstituted carbamoyl group having 1 to 20 carbon atoms, preferably 1 to 14 carbon atoms, a substituted or unsubstituted alkylthio group having 1 to 20 carbon atoms, preferably 1 to 14 carbon atoms, a substituted or unsubstituted arylthio group having 6 to 20 carbon atoms, preferably 6 to 14 carbon atoms, a substituted or unsubstituted (heterocyclyl)thio group having 1 to 20 carbon atoms, preferably 1 to 14 carbon atoms, a substituted or unsubstituted alkylsulfinyl group having 1 to 20 carbon atoms, preferably 1 to 14 carbon atoms, a substituted or unsubstituted arylsulfinyl group having 6 to 20 carbon atoms, preferably 6 to 14 carbon atoms, a substituted or unsubstituted (heterocyclyl)sulfinyl group having 1 to 20 carbon atoms, preferably 1 to 14 carbon atoms, a substituted or unsubstituted alkylsulfonyl group having 1 to 20 carbon atoms, preferably 1 to 14 carbon atoms, or a substituted or unsubstituted arylsulfonyl group having 6 to 20 carbon atoms, preferably 6 to 14 carbon atoms, a substituted or unsubstituted (heterocyclyl)sulfonyl group having 1 to 20 carbon atoms, preferably 1 to 14 carbon atoms, a nitro group, or a cyano group;
h, i, and j each is independently 0 or 1;
R^{x} and R^{y} each is independently a hydrogen atom, a silyl group, a metal atom, an ammonium moiety, or a phosphonium moiety, or R^{x} and R^{y} combine to form a metal atom or a silyl group; and
two or more of R¹, R², R³, R⁴, R⁵, R⁶, R⁷, R⁸, R⁹, R¹⁰, and R¹¹, or two or more of R¹, R², R³, R⁴, R⁵, R¹², and R¹³ may be linked with or without a heteroatom(s) to form any ring structure.

A halogen atom herein is a fluorine atom, a chlorine atom, a bromine atom, or an iodine atom.

The silyl group(s) for R^{x} and/or R^{y} above can include a R^{14′}R^{15′}R^{16′}Si group and other like silyl groups, in which R^{14′}, R^{15′}, and R^{16′} each is independently an alkyl group having 1 to 10 carbon atoms, preferably 1 to 4 carbon atoms, an aralkyl group having 7 to 10 carbon atoms, or an aryl group having 6 to 10 carbon atoms. The silyl group that R^{x} and R^{y} combine to form can include a —Si(R¹⁷)(R¹⁸)— group and other like silyl groups, in which R¹⁷ and R¹⁸ each is independently an alkyl group having 1 to 10 carbon atoms, preferably 1 to 4 carbon atoms, an aralkyl group having 7 to 10 carbon atoms, or an aryl group having 6 to 10 carbon atoms.

The metal atom(s) above can include an alkali metal atom, alkali earth metal atom, and/or transition metal atom. Among them, alkali metal atoms such as Li, Na, K, Rb, and Cs are preferable. Preferably when R^{x} and R^{y} combine to form a metal atom an alkali earth or transition metal atom such as Mg, Ca, Cu, and Zn atom is used.

The ammonium moieties above can include tetraalkylammonium such as tetramethylammonium, tetraethylammonium, tetrabutylammonium, and so on. The phosphonium moieties above can include tetraalkylphosphonium such as tetramethylphosphonium, tetraethylphosphonium, and so on, and tetraarylphosphonium such as tetraphenylphosphonium, tetra(tolyl)phosphonium, and so on.

The term “alkyl” as used herein refers to a linear, branched, or cyclic alkyl. The alkyl part of alkoxy, alkoxycarbonyl, alkylthio, alkylsulfinyl, or alkylsulfonyl group as used herein also refers to a linear, branched, or cyclic alkyl part. When an acyl or acyloxy group contains an alkyl part, the alkyl part is also linear, branched, or cyclic.

The term “substituted” as used in, for example, “substituted alkyl”, “substituted alkenyl”, “substituted alkynyl”, “substituted aryl”, “substituted heterocycly”, “substituted acyl”, “substituted alkoxycarbonyl”, “substituted aryloxycarbonyl”, “substituted (heterocyclyl)oxycarbonyl”, “substituted alkoxy”, “substituted aryloxy”, “substituted (heterocyclyl)oxy”, “substituted acyloxy”, “substituted amino”, “substituted carbamoyl”, “substituted alkylthio”, “substituted arylthio”, “substituted (heterocyclyl)thio”, “substituted alkylsulfinyl”, “substituted arylsulfinyl”, “substituted (heterocyclyl)sulfinyl”, “substituted alkylsulfonyl”, “substituted arylsulfonyl”, and “substituted (heterocyclyl)sulfonyl” herein means each basic moiety (alkyl, alkenyl, alkynyl, etc.) having one or more substituents such as a halogen atom, a substituted or unsubstituted alkyl group, a substituted or unsubstituted alkenyl group, a substituted or unsubstituted alkynyl group, a substituted or unsubstituted aryl group, a substituted or unsubstituted heterocyclyl group, and/or any other group with or without a heteroatom(s) such as an oxygen atom(s), a nitrogen atom(s), and/or a sulfur atom(s), wherein the substitution does not substantially limit reactions of this invention.

The term “heterocyclyl group” or “heterocyclyl” as used herein refers to univalent groups formed by removing a hydrogen atom from any ring atom of a heterocyclic compound which includes saturated as well as unsaturated heterocyclic compounds [see, Glossary of class names of organic compounds and reactive intermediates based on structure (IUPAC Recommendations 1995); Pure & Appl. Chem., Vol. 67 (Nos 8/9), pp 1307-1375 (1995)], incorporated herein by reference.

### Process I

The starting materials, i.e., oxygen-containing compounds having formula (II), are commercially available or can be prepared in accordance with understood principles of synthetic chemistry.

Illustrative oxygen-containing compounds of Process I, as presented by formula (II), include: diols, amino alcohols, silyl derivatives of diols and amino alcohols, and metal, ammonium, or phosphonium salts of diols and amino alcohols. These oxygen-containing compounds used for Process I include any stereoisomers such as diastereoisomers, enantioisomers, and racemates. Illustrative diols and their salts include, but are not limited to: ethylene glycol (1,2-ethanediol), LiOCH₂CH₂OH, NaOCH₂CH₂OH, KOCH₂CH₂OH, LiOCH₂CH₂OLi, NaOCH₂CH₂ONa, KOCH₂CH₂OK, (CH₃)₄NOCH₂CH₂OH, (C₄H₉)₄NOCH₂CH₂OH, (C₆H₅)₄POCH₂CH₂OH, 1,2-propanediol, LiOCH₂CH(OLi)CH₃, 1,3-propanediol, LiOCH₂CH₂CH₂OLi, NaOCH₂CH₂CH₂ONa, (C₄H₉)₄NOCH₂CH₂CH₂ON(C₄H₉)₄, 1,2-butanediol, 1,3-butanediol, 1,4-butanediol, 2,3-butanediol, 1,2-pentanediol, 1,3-pentandiol, 1,4-pentanediol, 1,5-pentanediol, 2,3-pentanediol, 2,4-pentanediol, 3-pentene-1,2-diol, 4-pentene-1,2-diol, 1,2-hexanediol, 1,2-heptanediol, 1,2-octanediol, 1,2-nonanediol, 1,2-decanediol, 1,2-undecanediol, 1,2-dodecanediol, 1,2-pentadecanediol, 1,2-hexadecanediol, 1,2-icosanediol, 1,2-docosanediol, 1-(cyclopropyl)-1,2-ethanediol, 1-(cyclopentyl)-1,2-ethanediol, 1-(cyclohexyl)-1,2-ethanediol, 1-phenyl-1,2-ethanediol, 1,2-diphenyl-1,2-ethanediol, 1-(α- or β-naphthyl)-1,2-ethanediol, 1-(α-, β- or γ-pyridyl)-1,2-ethanediol, 1-(α- or β-furyl)-1,2-ethanediol, 1-(α- or β-thienyl)-1,2-ethanediol, 1-(α- or β-pyrrolyl)-1,2-ethanediol, N-benzyl-2-(1′,2′-dihydroxyethyl)pyrrole, 1-(2′-quinolyl)-1,2-ethanediol, 3,4-epoxy-1,2-butandiol [(1′,2′-dihydroxylethyl)oxirane], 2-(1′,2′-dihydroxyethyl)aziridine, 2-(1′,2′-dihydroxyethyl)azetidine, 1-(imidazol-2′-yl)-1,2-ethanediol, 1-(1′-methylimidazol-2′-yl)-1,2-ethanediol, 1-(oxazol-2′-yl)-1,2-ethanediol, 1-(thiazol-2′-yl)-1,2-ethanediol, 1-(2′-, 4′-, or 5′-pyrimidyl)-1,2-ethanediol, 1-(2′-, 3′-, or 4′-piperidinyl)-1,2-ethanediol, 1-(2′- or 3′-pyrrolidinyl)-1,2-ethanediol, 1-(2′- or 3′-tetrahydrofuryl)-1,2-ethanediol, 1-(2′- or 3′-tetrahydrothienyl)-1,2-ethanediol, 1-(2′-, 3′-, or 4′-tetrahydropyranyl)-1,2-ethanediol, 1-(2′-, 3′-, or 4′-tetrahydrothiopyranyl)-1,2-ethanediol, 1-(2′- or 3′-morpholinyl)-1,2-ethanediol, 1-(2′-decahydroquinolinyl)-1,2-ethanediol, 1-phenyl-1,2-propanediol, 3-chloro-1,2-propanediol, 3-phenyl-1,2-propanediol, 3-methoxy-1,2-propanediol, 3-benzyloxy-1,2-propanediol, 3-phenoxy-1,2-propanediol, 3-acetyloxy-1,2-propanediol, 3-methylthio-1,2-propanediol, 3-phenylthio-1,2-propanediol, 3-methylsulfinyl-1,2-propanediol, 3-phenylsulfinyl-1,2-propanediol, 3-methylsulfonyl-1,2-propanediol, 3-phenylsulfonyl-1,2-propanediol, 2-methyl-1,3-propanediol, 2,2-dimethyl-1,3-propanediol, 2-cyclopentyl-1,3-propanediol, 2-allyl-1,3-propanediol, 2-phenyl-1,3-propanediol, 2-methoxy-1,3-propanediol, 2-benzyloxy-1,3-propanediol, 2-phenoxy-1,3-propanediol, 2-chloro-1,3-propanediol, 2-cyano-1,3-propanediol, 2-vinyl-1,3-propanediol, 2-ethynyl-1,3-propanediol, 2-bromo-2-nitro-1,3-propanediol, 2-methylthio-1,3-propanediol, 2-phenylthio-1,3-propanediol, 2-phenylsulfinyl-1,3-propanediol, 2-phenylsulfonyl-1,3-propanediol, 2-(2′-tetrahydrofuranyl)-1,3-propanediol, methyl 2,3-dihydroxypropanoate, ethyl 2,3-dihydroxybutanoate, dimethyl tartrate, di-tert-butyl tartrate, 2,3-dihydroxypropionitrile, 1,2-cyclopentanediol, 1,3-cyclopentanediol, 1,2-dihydroxy-4-cyclopentene, 1,2-cylcohexanediol, 1,3-cyclohexanediol, 1,4-cyclohexanediol, 1-phenylcyclohexane-1,2-diol, 2-cyclohexene-1,4-diol, 3-cyclohexene-1,2-diol, 4-cyclohexene-1,2-diol, 1,2-cycloheptanediol, 1,3-cycloheptanediol, 1,4-cycloheptanediol, 1,2-cyclooctanediol, 1,4-cyclooctanediol, 1,2-cyclononanediol, 1,2-cyclodecanediol, 1,2-cycloundenanediol, 1,2-cyclododecanediol, 3,4-dihydroxy-1-butene, 1,4-dihydroxy-2-butene, 3,4-dihydroxy-1-butyne, 1,2-dihydroxy-4-phenyl-3-betene, 1,2-dihydroxy-4-pentene, 3,5-dihydroxy-1-pentene, 1,2-dihydroxy-4-methyl-3-pentene, 1,2-dihydroxy-4-phenyl-3-pentene, 3,6-dihydroxy-1-hexene, 3,7-dihydroxy-1-heptene, 1,2-dihydroxy-1-vinyl-cyclopentane, 1,2-dihydroxy-1-vinyl-cyclohexane, 1,2-bis(hydroxymethyl)benzene, N-(tert-butoxycarbonyl)-3-hydroxy-4-(hydroxymethyl)pyrrolidine, N-(benzyloxycarbonyl)-3-hydroxy-4-(hydroxymethyl)pyrrolidine, N-(9-fluorenylmethyl)-3-hydroxy-4-(hydroxymethyl)pyrrolidine, cis-1,2-dihydro-1,2-naphthalenediol, 1,4-dihydro-1,4-naphthalenediol, estriol, dianhydro-D-glucitol, dianhydro-D-mannitol, and other like compounds.

Illustrative silyl derivatives of diols are exemplified by silyl derivatives of the diols listed above. Illustrative silyl derivatives of diols include, but are not limited to: (CH₃)₃SiOCH₂CH₂OSi(CH₃)₃, (C₂H₅)₃SiOCH₂CH₂OSi(C₂H₅)₃, ((CH₃)₃C)(CH₃)₂SiOCH₂CH₂OSi(CH₃)₂(C(CH₃)₃), (C₆H₅)(CH₃)₂SiOCH₂CH₂OSi(CH₃)₂(C₆H₅), (C₆H₅CH₂)(CH₃)₂SiOCH₂CH₂OSi(CH₃)₂(CH₂C₆H₅), (CH₃)₃SiOCH₂CH₂CH₂OSi(CH₃)₃, (CH₃)₃SiOCH₂CH₂CH₂OSi(CH₃)₃, (CH₃)₃SiOCH₂CH₂CH₂CH₂OSi(CH₃)₃, (CH₃)₃SiOCH₂CH₂CH₂CH₂CH₂OSi(CH₃)₃, CH₃CH(OSi(CH₃)₃)CH₂OSi(CH₃)₃, CH₃CH₂CH(OSi(CH₃)₃)CH₂OSi(CH₃)₃, C₆H₅CH(OSi(CH₃)₃)CH₂OSi(CH₃)₃, C₆H₅CH(OSi(CH₃)₃)CH(OSi(CH₃)₃)C₆H₅, CH₂═CHCH(OSi(CH₃)₃)CH₂OSi(CH₃)₃, CH≡CHCH(OSi(CH₃)₃)CH₂OSi(CH₃)₃,

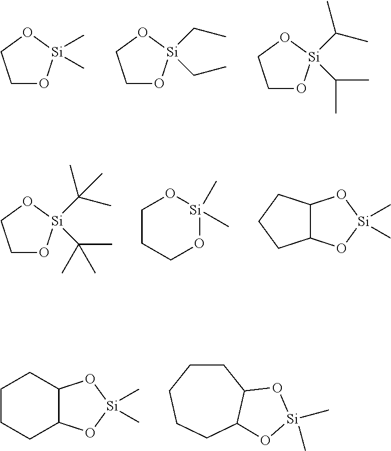

and other like compounds.

Illustrative amino alcohols and their salts include, but are not limited to: 2-amino-1-ethanol, LiOCH₂CH₂NH₂, NaOCH₂CH₂NH₂, KOCH₂CH₂NH₂, 2-methylamino-1-ethanol, LiOCH₂CH₂N(Li)CH₃, 2-ethylamino-1-ethanol, 2-propylamino-1-ethanol, 2-butylamino-1-ethanol, 2-phenylamino-1-ethanol, 2-benzylamino-1-ethanol, 2-acetylamino-1-ethanol, 2-benzoylamino-1-ethanol, 2-(methoxycarbonyl)amino-1-ethanol, 2-(tert-butoxycarbonylamino)-1-ethanol, 2-(9′-fluorenylmethoxycarbonyl)amino-2-ethanol, 2-(phenyloxycarbonyl)amino-1-ethanol, 2-(methylsulfonyl)amino-1-ethanaol, 2-(phenylsulfonyl)amino-1-ethanol, 2-benzylamino-1-phenyl-1-ethanol, 2-benzylamino-2-phenyl-1-ethanol, 1-amino-2-propanol, 1-benzylamino-2-propanol, 2-methylamino-1-propanol, 2-benzylamino-1-propanol, 3-benzyloxy-2-(tert-butoxycarbonylamino)-1-propanol, 2-benzylamino-1-butanol, 2-methylamino-1-butanol, 2-benzylamino-2-butanol, 3-benzylamino-1-butanol, 4-benzylamino-1-butanol, 4-methylamino-2-buten-1-ol, 4-benzylamino-2-buten-1-ol, 1-methylamino-2-hydroxy-3-butene, 1-benzylamino-2-hydroxy-3-butene, 1-methylamino-2-hydroxy-4-phenyl-3-butene, 2-amino-1-pentanol, 2-methylamino-1-pentanol, 2-benzylamino-1-pentanol, 1-methylamino-2-penten-4-ol, 1-ethylamino-2-hydroxy-3-pentene, 1-benzylamino-2-hydroxy-4-methyl-3-pentene, 2-benzylamino-1-hexanol, 2-benzylamino-heptanol, 2-benzylamino-1-octanol, 2-benzylamino-1-nonanol, 2-benzylamino-1-decanol, 2-methylamino-1-phenyl-1-ethanol, 1-cyclopentyl-2-methylamino-1-ethanol, 1-cyclohexyl-2-methylamino-1-ethanol, 1-vinyl-2-methyl amino-1-ethanol, 1-(N,N-dimethylcarbamonyl)-2-methylamino-1-ethanol, 2-benzylamino-1-cyclopentanol, 2-phenylamino-1-cyclopentanol, 3-benzylamino-1-cyclopentanol, 2-methylamino-1-cyclopentanol, 2-benzylamino-1-hydroxymethyl-cyclopentane, 4-methylamino-2-cyclopenten-1-ol, 2-methylamino-1-cyclohexanol, 2-benzylamino-1-cyclohexanol, 2-phenylamino-1-hydroxymethyl-cyclohexane, 4-ethylamino-2-cyclohexne-1-ol, 2-benzylamino-1-cycloheptane, 2-hydroxyazetidine, 3-pyrrolidinol, 2-pyrrolidinemethanol, 3-hydroxypiperidine, 4-hydroxypiperidine, 2-piperidinemethanol, 3-piperidinemethanol, 4-piperidinemethanol, 2-piperidineethanol, 3-piperidineethanol, 4-piperidineethanol, 1-piperazinepropanol, 1-(2-hydroxyethyl)piperazine, phenylephrine, and other like compounds.

Illustrative silyl derivatives of amino alcohols are exemplified by silyl derivatives of the amino alcohols listed above. Illustrative silyl derivatives of amino alcohols include, but are not limited to: (CH₃)₃SiNHCH₂CH₂OSi(CH₃)₃, ((CH₃)₃Si)₂NCH₂CH₂OSi(CH₃)₃, NH₂CH₂CH₂OSi(CH₃)₃, CH₃N(Si(CH₃)₃)CH₂CH₂OSi(CH₃)₃, CH₃N(Si(CH₂CH₃)₃)CH₂CH₂OSi(CH₂CH₃)₃, CH₃N(Si(CH₃)₂(tert-C₄H₉))CH₂CH₂OSi((CH₃)₂(tert-C₄H₉)), CH₃N(Si(CH₃)₂(C₆H₅))CH₂CH₂OSi((CH₃)₂(C₆H₅)), CH₃NHCH₂CH₂OSi(CH₃)₃, C₆H₅CH₂N(Si(CH₃)₃)CH₂CH₂OSi(CH₃)₃, CH₃N(Si(CH₃)₃)CH(CH₃)CH₂OSi(CH₃)₃, CH₃CH(OSi(CH₃)₃)CH₂N(Si(CH₃)₃)CH₃, C₆H₅CH₂N(Si(CH₃)₃)CH(CH₂CH₃)CH₂OSi(CH₃)₃, CH₃CH₂CH(OSi(CH₃)₃)CH₂N(Si(CH₃)₃)CH₂C₆H₅,

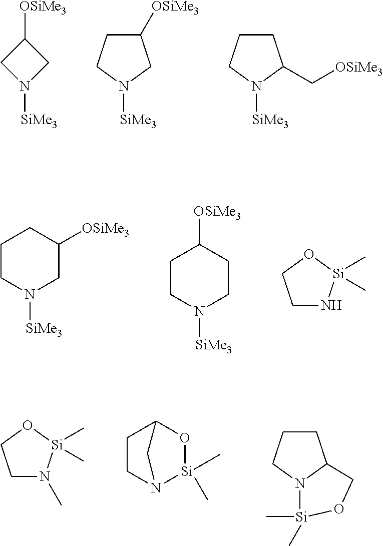

and other like compounds.

For each of R¹⁻⁴, a hydrogen atom, a cyano group, or a substituted or unsubstituted alkyl, alkenyl, alkynyl, aryl, alkoxy, acyl, alkoxycarbonyl, or aryloxycarbonyl group is preferable because of availability. For R⁵, a hydrogen atom or a substituted or unsubstituted alkyl, aryl, acyl, alkoxycarbonyl, or aryloxycarbonyl group is preferable because of availability. For each of R⁶⁻¹³, a hydrogen atom, a halogen atom, a nitro group, a cyano group, or a substituted or unsubstituted alkyl, alkenyl, alkynyl, aryl, alkoxy, aryloxy, acyl, acyloxy, alkoxycarbonyl, or aryloxycarbonyl group is preferable because of availability.

Illustrative arylsulfur trifluorides of Process I, as represented by formula (III), can be prepared as described in the literature [see J. Am. Chem. Soc., Vol. 82 (1962), pp. 3064-3072; Synthetic Communications, Vol. 33 (2003), pp. 2505-2509; and U.S. Pat. No. 7,265,247 B1 and U.S. Pat. No. 7,381,846 B2, each of which is incorporated by reference herein for all purposes]. Arylsulfur trifluorides have high thermal stability (see U.S. Pat. No. 7,381,846 B2). The arylsulfur trifluorides are exemplified, but are not limited by, phenylsulfur trifluoride, each isomer (o, m, and p-isomers) of methylphenylsulfur trifluoride, each isomer of dimethylphenylsulfur trifluoride, each isomer of trimethylphenylsulfur trifluoride, each isomer of ethylphenylsulfur trifluoride, each isomer of n-propylphenylsulfur trifluoride, each isomer of isopropylphenylsulfur trifluoride, each isomer of n-butylphenylsulfur trifluoride, each isomer of isobutylphenylsulfur trifluoride, each isomer of sec-butylphenylsulfur trifluoride, each isomer of (tert-butyl)phenylsulfur trifluoride, each isomer of di(isopropyl)phenylsulfur trifluoride, each isomer of tri(isopropyl)phenylsulfur trifluoride, each isomer of (tert-butyl)dimethylphenylsulfur trifluoride, each isomer of (methoxymethyl)phenylsulfur trifluoride, each isomer of bis(methoxymethyl)phenylsulfur trifluoride, each isomer of bis(methoxymethyl)-(tert-butyl)phenylsulfur trifluoride, each isomer of bis(ethoxymethyl)-(tert-butyl)phenylsulfur trifluoride, each isomer of bis(isopropoxymethyl)-(tert-butyl)phenylsulfur trifluoride, each isomer of fluorophenylsulfur trifluoride, each isomer of chlorophenylsulfur trifluoride, each isomer of bromophenylsulfur trifluoride, each isomer of iodophenylsulfur trifluorode, each isomer of difluorophenylsulfur trifluoride, each isomer of trifluorophenylsulfur trifluoride, each isomer of tetrafluorophenylsulfur trifluoride, pentafluorophenylsulfur trifluoride, each isomer of dichlorophenylsulfur trifluoride, each isomer of dibromophenylsulfur trifluoride, each isomer of chlorofluorophenylsulfur trifluoride, each isomer of bromofluorophenylsulfur trifluoride, each isomer of chloro(methyl)phenylsulfur trifluoride, each isomer of chloro(dimethyl)phenylsulfur trifluoride, each isomer of nitrophenylsulfur trifluoride, each isomer of dinitrophenylsulfur trifluoride, each isomer of cyanophenylsulfur trifluoride, and other like compounds. Among them, phenylsulfur trifluoride, 4-(tert-butyl)-2,6-dimethylphenylsulfur trifluoride, p-methylphenylsulfur trifluoride, p-fluorophenylsulfur trifluoride, p-chlorophenylsulfur trifluoride, p-bromophenylsulfur trifluoride, o- and p-nitrophenylsulfur trifluoride, p-(tert-butyl)phenylsulfur trifluoride, 2,6-bis(methoxymethyl)phenylsulfur trifluoride, 2,6-bis(methoxymethyl)-4-(tert-butyl)phenylsulfur trifluoride, and 2,4,6-tri(isopropyl)phenylsulfur trifluoride are preferable because of availability and cost considerations.

The reaction of an oxygen-containing compound of formula (II), in which at least one of R^{x} and R^{y} is a hydrogen atom, with an arylsulfur trifluoride of formula (III) may be conducted in the presence of a base, which may increase the yield of the product. In particular, when an oxygen-containing compound of formula (II), in which A=NR⁵ and R^{x}=H and/or R^{y}=H, is used, a base is preferable. Preferable bases are exemplified by amines such as trimethylamine, triethylamine, tripropylamine, isopropyldimethylamine, N,N-diisopropylethylamine, tributylamine, 1,8-diazobicyclo[5.4.0]undec-7-ene, 1,4-diazobicyclo[4.3.0]non-5-ene, 1,4-diazobicyclo[2.2.2]octane, pyridine, methylpyridine, dimethylpyridine, trimethylpyridine, (N,N-dimethylamino)pyridine, quinoline, isoquinoline, and other like compounds; carbonates such as sodium carbonate, sodium bicarbonate, potassium carbonate, potassium bicarbonate, lithium carbonate, lithium bicarbonate, and other like compounds; fluorides such as lithium fluoride, sodium fluoride, potassium fluoride, cesium fluoride, tetramethylammonium fluoride, tetraethyammonium fluoride, tetrabutylammonium fluoride, and other like compounds. The amount of base used for a reaction of this type can preferably be selected from about 0.5 mol base to about 5 mol base per 1 mol of an oxygen-containing compound of formula (II).

When an oxygen-containing compound of formula (II), in which at least one of R^{x} and R^{y} is a silyl group, or R^{x} and R^{y} combine to form a silyl group, is used, the reaction of the oxygen-containing compound with an arylsulfur trifluoride of formula (III) can preferably be conducted in the presence of a silicon atom-activating agent. Illustrative silicon atom-activating agents include: fluorides containing a fluoride anion such as potassium fluoride, cesium fluoride, tetramethylammonium fluoride, tetraethylammonium fluoride, tetrabutylammonium fluoride, and other like fluorides; oxide salts such as sodium methoxide, potassium methoxide, sodium ethoxide, potassium ethoxide, potassium tert-butoxide, and other like oxides; cyanide salts such as sodium cyanide, potassium cyanide, tetraethylammonium cyanide, tetrabutylammonium cyanide, and other like cyanides. Among these, fluorides are preferable because of high yield of product(s). The amount of silicon atom-activating agent used for a reaction can preferably be selected from a catalytic amount to an amount in excess. A catalytic amount is preferable because of cost and yield.

The reaction of an oxygen-containing compound of formula (II), in which A is an oxygen atom, with an arylsulfur trifluoride of formula (III) may be conducted in the presence of hydrogen fluoride or a mixture of hydrogen fluoride and an amine compound(s), which may increase the yield of the product. The hydrogen fluoride may be in situ generated by addition of a necessary amount of water, an alcohol such as methanol, ethanol, propanol, isopropanol, butanol, sec-butanol, isobutanol, tert-butanol and so on, or a carboxylic acid such as acetic acid, propionic acid, and so on. The water, alcohol, or carboxylic acid is added into the reaction mixture, since an arylsulfur trifluoride (ArSF₃) reacts with water, an alcohol, or a carboxylic acid to generate hydrogen fluoride, as shown in the following reaction equations, however, this in situ generation method of hydrogen fluoride requires ArSF₃ be consumed at equimolar amounts of water, an alcohol, or a carboxylic acid.

ArSF₃+H₂O→2HF+ArSOF;

or

ArSF₃+CₘH₂ₘ₊₁OH_{(m=1˜4)}→HF+CₘH₂ₘ₊₁F_{(m=1˜4)}+ArSOF;

or

ArSF₃+CₘH₂ₘ₊₁COOH_{(m=1˜4)}→HF+CₘH₂ₘ₊₁COF_{(m=1˜4)}+ArSOF.

The mixture of hydrogen fluoride and amine compound(s) is preferably exemplified by a mixture of hydrogen fluoride and pyridine (for example, a mixture of about 70 wt % HF and about 30 wt % pyridine) or a mixture of hydrogen fluoride and triethylamine [for example, a 3:1 (molar ratio) mixture of hydrogen fluoride and triethylamine, Et₃N(HF)₃]. The amount of hydrogen fluoride or a mixture of hydrogen fluoride and an amine compound(s) may be a catalytic amount to an amount in excess for the reaction of this invention, dependent on reaction conditions.

The reaction of an oxygen-containing compound of formula (II), in which A is an oxygen atom, with an arylsulfur trifluoride of formula (III) can also be conducted in the presence of a tetraalkylammonium fluoride-hydrogen fluoride such as tetrabutylammonium fluoride-hydrogen fluoride [for example, tetrabutylammonium dihydrogentrifluoride, (C₄H₉)₄NH₂F₃]. The amount of a tetraalkylammonium fluoride-hydrogen fluoride may be a catalytic amount to an amount in excess for the reaction of this invention, dependent on reaction conditions.

Process I can be carried out in the presence of one or more solvents or in the absence of solvent. The use of solvent is preferable for mild and efficient reactions. When solvent is used, a preferable solvent will not substantially react with the starting material(s) and/or reagents, the intermediates, and/or the final product(s). Suitable solvents include, but are not limited to: alkanes, halocarbons, ethers, nitriles, aromatics, nitro compounds, esters, and so on, and mixtures thereof. Example alkanes include normal, branched, cyclic isomers of pentane, hexane, heptane, octane, nonane, decane, dodecane, undecane, and other like compounds. Illustrative halocarbons include: dichloromethane, chloroform, carbon tetrachloride, dichloroethane, trichloroethane, terachloroethane, trichlorotrifluoroethane, chlorobenzene, dichlorobenzene, trichlorobenzene, hexafluorobenzene, benzotrifluoride, and bis(trifluoromethyl)benzene; normal, branched, cyclic isomers of perfluoropentane, perfluorohexane, perfluoroheptane, perfluorooctane, perfluorononane, and perfluorodecane; perfluorodecalin; and other like compounds. Illustrative ethers include: diethyl ether, dipropyl ether, di(isopropyl)ether, dibutyl ether, tert-butyl methyl ether, dioxane, glyme (1,2-dimethoxyethane), diglyme, triglyme, and other like compounds. Illustrative nitriles include: acetonitrile, propionitrile, benzonitrile, and other like compounds. Illustrative aromatics include: benzene, toluene, xylene, and other like compounds. Illustrative nitro compounds include: nitromethane, nitroethane, nitrobenzene, and other like compounds. Illustrative esters include: methyl acetate, methyl propionate, ethyl acetate, ethyl propionate, propyl acetate, isopropyl acetate, butyl acetate, isobutyl acetate, sec-butyl acetate, and other like compounds.

In order to obtain good yield of product in Process I, the reaction temperature can preferably be selected in the range of about −100° C.˜+200° C.; more preferably, the reaction temperature can be selected in the range of about −80° C.˜+150° C.; and furthermore preferably, the reaction temperature can be selected in the range of about −80° C.˜+120° C.

Reaction conditions of Process I are optimized to obtain economically good yields of product. In one illustrative embodiment, from about 0.5 mol to about 2 mol, more preferably, from about 0.8 mol to about 1.5 mol, furthermore preferably about 0.9 to about 1.2 mol of arylsulfur trifluoride (formula III) are combined with 1 mol of oxygen-containing compound (formula II) to obtain a good yield of fluoroalkyl arylsulfinyl compound (formula I).

Note that the reaction time for Process I varies dependent upon reaction temperature, and the types and amounts of substrates, reagents, and solvents. As such, reaction time is generally determined as the amount of time required to complete a particular reaction, but can be from about 1 minute to about several days, preferably, within a few days.

R¹, R², R³, R⁴, R⁵, R⁶, R⁷, R⁸, R⁹, R¹⁰, R¹¹, R¹² and/or R¹³ of the products represented by the formula (I) may be different from R¹, R², R³, R⁴, R⁵, R⁶, R⁷, R⁸, R⁹, R¹⁰, R¹¹, R¹² and/or R¹³ of the starting materials represented by the formula (II). Thus, embodiments of this invention include transformation of the R¹, R², R³, R⁴, R⁵, R⁶, R⁷, R⁸, R⁹, R¹⁰, R¹¹, R¹² and/or R¹³ to different R¹, R², R³, R⁴, R⁵, R⁶, R⁷, R⁸, R⁹, R¹⁰, R¹¹, R¹² and/or R¹³ which may take place during the reaction of the present invention or under the reaction conditions as long as both the fluorination and the arylsulfinylation take place.

The present invention also includes a method wherein the arylsulfur trifluoride of formula (III) used in Process 1 (Scheme 1) is prepared by the method comprising reacting arylsulfur halotetrafluoride of formula (IV) with a reducing substance. Thus, embodiments of the present invention provide a method (Scheme 2, Processes II and Ia) for preparing a fluoroalkyl arylsulfinyl compound having a formula (I), which comprises (Process II) reacting an arylsulfur halotetrafluoride having a formula (IV) with a reducing substance that reduces the arylsulfur halotetrafluoride and (Process Ia) reacting a resulting arylsulfur trifluoride having a formula (III) with an oxygen-containing compound having a formula (II):

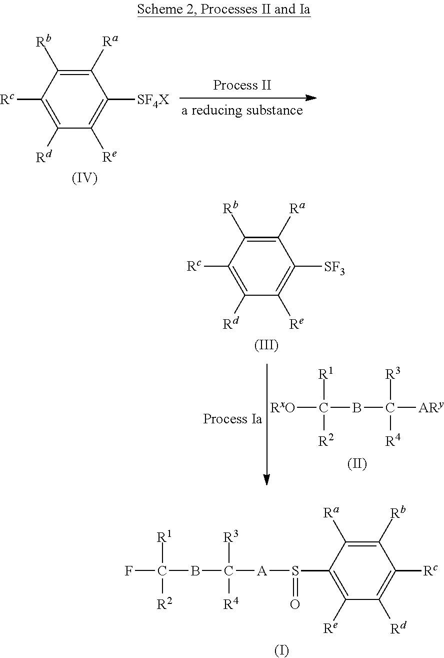

For the compounds represented by formulas (I), (II), (III), and (IV), R¹, R², R³, R⁴, R^{x}, R^{y}, A, B, R^{a}, R^{b}, R^{c}, R^{d}, and R^{e} are the same as described above. X is a chlorine atom, a bromine atom, or an iodine atom. Preferably X is a chlorine atom to minimize cost.

### Process II

The starting materials, arylsulfur halotetrafluorides used for Process II, may be prepared according to the method shown in the literature (see Can. J. Chem., Vol. 75, pp. 1878-1884, incorporated herein by reference for all purposes). Arylsulfur halotetrafluorides can be prepared industrially at low cost using the methods shown in Examples 47˜62 (as well as methodologies provided throughout the disclosure).

Note that according to the nomenclature of Chemical Abstract Index Name, and in accordance with the present disclosure, for example, C₆H₅—SF₄Cl is named sulfur, chlorotetrafluorophenyl-; p-CH₃—C₆H₄—SF₄Cl is named sulfur, chlorotetrafluoro(4-methylphenyl)-; and p-NO₂—C₆H₄—SF₄Cl is named sulfur, chlorotetrafluoro(4-nitrophenyl)-.

Arylsulfur halotetrafluoride compounds of formula (IV) include isomers such as trans-isomers and cis-isomers as shown below; arylsulfur halotetrafluoride is represented by ArSF₄X:

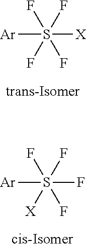

Illustrative arylsulfur halotetrafluorides include: phenylsulfur chlorotetrafluoride, each isomer (o, m, and p-isomers) of methylphenylsulfur chlorotetrafluoride, each isomer of dimethylphenylsulfur chlorotetrafluoride, each isomer of ethylphenylsulfur chlorotetrafluoride, each isomer of n-propylphenylsulfur chlorotetrafluoride, each isomer of isopropylphenylsulfur chlorotetrafluoride, each isomer of n-butylphenylsulfur chlorotetrafluoride, each isomer of sec-butylphenylsulfur chlorotetrafluoride, each isomer of isobutylphenylsulfur chlorotetrafluoride, each isomer of (tert-butyl)phenylsulfur chlorotetrafluoride, each isomer of fluorophenylsulfur chloroteterafluoride, each isomer of chlorophenylsulfur chlorotetrafluoride, each isomer of bromophenylsulfur chlorotetrafluoride, each isomer of iodophenylsulfur chlorotetrafluoride, each isomer of difluorophenylsulfur chlorotetrafluoride, each isomer of trifluorophenylsulfur chlorotetrafluoride, pentafluorophenylsulfur chlorotetrafluoride, each isomer of nitrophenylsulfur chlorotetrafluoride, and so on. Among them, phenylsulfur chlorotetrafluoride, p-methylphenylsulfur chlorotetrafluoride, p-(tert-butyl)phenylsulfur chlorotetrafluoride, p-chlorophenylsulfur chlorotetrafluoride, p-fluorophenylsulfur chlorotetrafluoride, p-bromophenylsulfur chlorotetrafluoride, and p-nitrophenylsulfur chlorotetrafluoride are preferable so as to minimize cost.

A reducing substance used in Process II is: 1) an element or an organic or inorganic compound which reduces an arylsulfur halotetrafluoride of the formula (I) used in the reaction; or 2) of which reduction potential is lower than that of arylsulfur halotetrafluoride of the formula (I) used in the reaction. One or more reducing substances can be used in a reaction, including mixtures thereof.

Reducing substances herein include elements such as: metals such as alkali metals (elements in Group 1 of the Periodic Table), alkali earth metals (elements in Group 2 of the Periodic Table), transition metals and inner transition metals (elements in Groups 3˜12 of the Perioidic Table), and metals in Groups 13˜15 of the Periodic Table such as Al, Ga, In, Tl, Sn, Pb, and Bi; semi-metals such as B, Si, Ge, As, Sb, Te, Po, and At; nonmetal elements in Groups 13˜17 of the Periodical Table (C, P, S, Se, I, and so on). Among these, preferred elements are alkali metals, alkali earth metals, transition metals, metals in Groups 13˜15 of the Periodic Table, semi-metals, and nonmetals.

Reducing substances herein also include inorganic compounds such as: hydrogen, metal compounds, semi-metal compounds, and nonmetal compounds. Among these, preferred inorganic compounds include: metal salts, semi-metal salts, nonmetal salts, inorganic chloride salts, inorganic bromide salts, inorganic iodide salts, ammonia (NH₃), inorganic sulfur compounds, and so on.

Preferred inorganic chloride salts are exemplified with metal chlorides (LiCl, NaCl, KCl, RbCl, CsCl, MgCl₂, MgClF, CaCl₂, TiCl₂, VCl₂, CrCl₂, FeCl₂, CuCl, SnCl₂, and other metal salts containing chloride anions), ammonium chloride, and other inorganic salts containing chloride anions. Preferred inorganic bromide salts are exemplified with metal bromides (LiBr, NaBr, KBr, RbBr, CsBr, MgBr₂, MgBrCl, MgBrF, CaBr₂, FeBr₂, CuBr, SnBr₂, and other metal salts containing bromide anions), ammonium bromide, and other inorganic salts containing bromide anions. Preferred inorganic iodide salts are exemplified with metal iodides (LiI, NaI, KI, RbL, CsI, MgI₂, MgBrI, MgClI, MgFI, CaI₂, FeI₂, CuI, SnI₂, and other metal salts containing iodide anions), ammonium iodide, and other inorganic salts containing iodide anions. Preferred inorganic sulfur compounds are exemplified with hydrogen sulfide, salts of hydrogen sulfide, salts of sulfide, salts of hydrogen sulfite, salts of sulfite, salts of thiosulfate, salts of thiocyanate, and other inorganic compounds containing sulfur (valence state II or IV).

Among these, more preferred inorganic compounds include: inorganic chloride salts, inorganic bromide salts, and inorganic iodide salts.

Preferred reducing substances also include organic compounds such as: organic chloride salts, organic bromide salts, organic iodide salts, substituted and unsubstituted arenes, substituted and unsubstituted heteroarenes, substituted and unsubstituted unsaturated aliphatic hydrocarbons, substituted and unsubstituted nitrogen-containing aliphatic hydrocarbons, salts or complexes of substituted or unsubstituted heteroarenes and hydrogen fluoride (HF), salts or complexes of substituted or unsubstituted nitrogen-containing aliphatic hydrocarbons and hydrogen fluoride (HF), organic sulfur compounds, organic selenium compounds, organic phosphorous compounds, and so on.

Preferred organic chloride salts are exemplified with methylammonium chloride, dimethylammonium chloride, trimethylammonium chloride, tetramethylammonium chloride, ethylammonium chloride, diethylammonium chloride, triethylammonium chloride, tetraethylammonium chloride, propylammonium chloride, tripropylammonium chloride, tetrapropylammonium chloride, butylammonium chloride, tributylammonium chloride, tetrabutylammonium chloride, anilinium chloride, N,N-dimethylanilinium chloride, pyridinium chloride, N-methylpyridinium chloride, pyrrolidinium chloride, piperidinium chloride, and other organic salts containing chloride anions.

Preferred organic bromide salts are exemplified with methylammonium bromide, dimethylammonium bromide, trimethylammonium bromide, tetramethylammonium bromide, triethylammonium bromide, tetraethylammonium bromide, tripropylammonium bromide, tributylammonium bromide, tetrabutylammonium bromide, pyridinium bromide, and other organic salts containing bromide anions.

Preferred organic iodide salts are exemplified with methylammonium iodide, dimethylammonium iodide, trimethylammonium iodide, tetramethylammonium iodide, triethylammonium iodide, tetraethylammonium iodide, tributylammonium iodide, tetrabutylammonium iodide, pyridinium iodide, and other organic salts containing iodide anions.

Preferred substituted and unsubstituted arenes are exemplified with benzene, toluene, xylene, mesitylene, durene, hexamethylbenzene, anisole, dimethoxybenzene, aniline, N,N-dimethylaniline, phenylenediamine, phenol, salts of phenol, hydrobenzoquinone, naphthalene, indene, anthracene, phenanthrene, pyrene, and so on.

Preferred substituted and unsubstituted heteroarenes are exemplified with pyridine, methylpyridine, dimethylpyridine, trimethylpyridine, fluoropyridine, chloropyridine, dichloropyridine, pyrrole, indole, quinoline, isoquinoline, carbazole, imidazole, pyrimidine, pyridazine, pyrazine, triazole, furan, benzofuran, thiophene, benzothiophene, thiazole, phenothiazine, phenoxazine, and so on.

Preferred substituted and unsubstituted unsaturated aliphatic hydrocarbons are exemplified with substituted and unsubstituted alkenes such as ethylene, propene, butene, isobutylene, 2-methyl-2-butene, 2,3-dimethyl-2-butene, 2,3-dimethyl-1-butene, butadiene, pentene, 2-methyl-1-pentene, 2-methyl-2-pentene, hexene, cyclohexene, 1-methyl-1-cyclohexene, 1,2-dimethyl-1-cyclohexene, 2-N,N-diethylamino-1-propene, 1-N,N-dimethylamino-1-cyclohexene, 1-N,N-diethylamino-1-cyclohexene, 1-pyrrolidino-1-cyclohexene, 1-pyrrolidino-1-cyclopentene, styrene, α- and β-methylstyrene, stilbene, 2-methoxy-1-propene, ethyl vinyl ether, 2,3-dihydrofuran, 2,3-dihydro-5-methylfuran, 3,4-dihydro-2H-pyran, and so on, and substituted and unsubstituted alkynes such as acetylene, propyne, phenylacetylene, diphenylacetylene, and so on.

Preferred substituted and unsubstituted nitrogen-containing aliphatic hydrocarbons are exemplified with methylamine, ethylamine, diethylamine, triethylamine, propylamine, butylamine, pyrrolidine, N-methylpyrrolidine, piperidine, N-methylpiperidine, morpholine, N-methylmorpholine, ethylenediamine, N,N,N′N′-tetramethylethylenediamine, triethylenediamine, urea, tetramethylurea, and so on.

Preferred salts or complexes of substituted or unsubstituted heteroarenes and hydrogen fluoride (HF), are exemplified with pyridine.HF, pyridine.2HF, pyridine.3HF, methylpyridine.HF, dimethylpyridine.HF, trimethylpyridine.HF, and so on.

Preferred salts or complexes of substituted or unsubstituted nitrogen-containing aliphatic hydrocarbons and hydrogen fluoride (HF), are exemplified with triethylamine.HF, triethylamine.2HF, triethylamine-3HF, trimethylamine.HF, and so on.

Preferred organic sulfur compounds are exemplified with organic sulfides, organic disulfides, organic polysulfides, organic sulfenyl halides, and organic thiols and their salts.

Preferred organic sulfides are exemplified with dimethyl sulfide, diethyl sulfide, dipropyl sulfide, dibutyl sulfide, di(tert-butyl) sulfide, tetrahydrothiophene, methyl phenyl sulfide, trimethylsilyl phenyl sulfide, diphenyl sulfide, bis(o, m, and p-methylphenyl) sulfides, bis(o, m, and p-fluorophenyl) sulfides, bis(o, m, and p-chlorophenyl) sulfide, bis(o, m, and p-bromophenyl) sulfide, bis(o, m, and p-nitrophenyl) sulfide, and so on.

Preferred organic disulfides are exemplified with dimethyl disulfide, diethyl disulfide, diisopropyl disulfide, di(tert-butyl) disulfide, diphenyl disulfide, bis(o, m, and p-methylphenyl) disulfides, bis(o, m, and p-ethylphenyl) disulfide, bis(o, m, and p-n-propylphenyl) disulfide, bis(o, m, and p-isopropylphenyl) disulfide, bis(o, m, and p-butylphenyl) disulfide, bis(o, m, and p-isobutylphenyl) disulfide, bis(o, m, and p-sec-butylphenyl) disulfide, bis(o, m, and p-tert-butylphenyl) disulfide, each isomer of bis(dimethylphenyl) disulfide, each isomer of bis(trimethylphenyl) disulfide, bis(4-tert-butyl-2,6-dimethylphenyl) disulfide, bis(o, m, and p-fluorophenyl) disulfides, bis(o, m, and p-chlorophenyl) disulfide, bis(o, m, and p-bromophenyl) disulfide, bis(o, m, and p-iodophenyl) disulfides, bis(o, m, and p-nitrophenyl) disulfide, and so on.

Preferred organic polysulfides are exemplified with diphenyl trisulfide, dimethyl trisulfide, and so on.

Preferred organic sulfenyl halides are exemplified with phenylsulfenyl fluoride, phenylsulfenyl chloride, o, m, and p-fluorophenylsulfenyl chloride, o, m, and p-chlorophenylsulfenyl chloride, o, m, and p-bromophenylsulfenyl chloride, o, m, and p-nitrophenylsulfenyl chloride, and so on.

Preferred organic thiols and their salts are exemplified with methanethiol, ethanethiol, propanethiol, isopropanethiol, butanethiol, sec-butanethiol, isobutanethiol, tert-butanethiol, thiophenol, o, m, and p-methylbenzenethiols, o, m, and p-ethylbenzenethiol, o, m, and p-n-propylbenzenethiol, o, m, and p-isopropylbenzenethiol, o, m, and p-butylbenzenethiol, o, m, and p-isobutylbenzenethiol, o, m, and p-sec-butylbenzenethiol, o, m, and p-tert-butylbenzenethiol, each isomers of dimethylbenzenethiol, each isomer of trimethylbenzenethiol, 4-tert-butyl-2,6-dimethylbenzenethiol, o, m, and p-chlorobenzenethiols, o, m, and p-fluorobenzenethiols, o, m, and p-bromobenzenethiols, o, m, and p-nitrobenzenethiol, and metal salts, ammonium salts, phosphonim salts of these organic thiols.

Preferred organic selenium compounds are exemplified with benzeneselenol, diphenyl selenide, diphenyl diselenide, and so on.

Preferred organic phosphorous compounds are exemplified with trimethylphosphine, triethylphosphine, tripropylphosphine, tributylphosphine, triphenylphosphine, trimethylphosphite, triethylphosphite, tripropylphosphite, tributylphosphite, triphenylphosphite, and so on.

Preferred reducing substances in general include: the elements such as alkali metals, alkali earth metals, transition metals, metals in Groups 13˜15 of the Periodic Table, and semi-metals, the inorganic compounds such as inorganic chloride salts, inorganic bromide salts, and inorganic iodide salts, and the organic compounds such as organic chloride salts, organic bromide salts, organic iodide salts, substituted and unsubstituted arenes, substituted and unsubstituted heteroarenes, substituted and unsubstituted unsaturated aliphatic hydrocarbons, substituted and unsubstituted nitrogen-containing aliphatic hydrocarbons, organic sulfur compounds, salts or complexes of substituted or unsubstituted heteroarenes and hydrogen fluoride, and salts or complexes of substituted or unsubstituted nitrogen-containing aliphatic hydrocarbons and hydrogen fluoride.

Among the reducing substances exemplified above, substituted or unsubstituted heteroarenes are preferable, and among these, substituted or unsubstituted pyridines such as pyridine, methylpyridine, dimethylpyridine, trimethylpyridine, chlororpyridine and other like pyridines are more preferable; pyridine is furthermore preferable due to its low cost, mild reactions, and high yields of arylsulfur trifluoride of formula (III) and of the final products of formula (I).

Process II can be carried out in the presence of one or more solvents or in the absence of solvent. In some cases, the use of solvent is preferable for mild and efficient reactions. When solvent is used, the preferable solvents will not substantially react with the starting materials and reagents, the intermediates, and/or the final products. Suitable solvents include the same solvents as were discussed in Process I above.

In order to obtain good yield of product in Process II, the reaction temperature is preferably selected in the range of about −100° C.˜+200° C.; more preferably, the reaction temperature is selected in the range of about −80° C.˜+150° C.; and furthermore preferably, the reaction temperature is selected in the range of about −80° C.˜+120° C.

Reaction conditions of Process II are optimized to obtain economically good yield of product. The amount of a reducing substance greatly varies dependent on the nature and reactivity of the reducing substance. However, in one illustrative embodiment, from about 0.1 mol to about 5 mol, more preferably, from about 0.15 mol to about 3 mol of a reducing substance can be selected against 1 mol of arylsulfur halotetrafluoride (formula IV) to obtain a good yield of arylsulfur trifluoride (formula III).

Note that the reaction time for Process II varies dependent upon reaction temperature, and the types and amounts of substrates, reagents, and solvents. As such, reaction time is generally determined as the amount of time required to complete a particular reaction, but can be from about 1 minute to several days, preferably, within a few days.

### Process Ia

Process Ia is the same as Process I except that arylsulfur trifluoride of formula (III) as used in Process Ia is prepared by Process II.

Illustrative oxygen-containing compounds, represented by formula (II), used in Process II are the same as exemplified in Process I above.

It is preferable that the arylsulfur trifluoride prepared by Process II can be used without isolation for the next process, Process Ia. Accordingly, the reaction mixture obtained in Process II can be used for the next process, Process Ia. In this case, the use of a silyl activating catalyst such as potassium fluoride and tetrabutylammonium fluoride is not necessary when an oxygen-containing compound of formula (II) (R^{x} and R^{y} each is a silyl group or R^{x} and R^{y} combine to form a silyl group) is used for this process (Process Ia).

R¹, R², R³, R⁴, R⁵, R⁶, R⁷, R⁸, R⁹, R¹⁰, R¹¹, R¹² and/or R¹³ of the products represented by the formula (I) may be different from R¹, R², R³, R⁴, R⁵, R⁶, R⁷, R⁸, R⁹, R¹⁰, R¹¹, R¹² and/or R¹³ of the starting materials represented by the formula (II). Thus, embodiments of this invention include transformation of the R¹, R², R³, R⁴, R⁵, R⁶, R⁷, R⁸, R⁹, R¹⁰, R¹¹, R¹² and/or R¹³ to different R¹, R², R³, R⁴, R⁵, R⁶, R⁷, R⁸, R⁹, R¹⁰, R¹¹, R¹² and/or R¹³ which may take place during the reaction of the present invention or under the reaction conditions as long as both the fluorination and the arylsulfinylation take place.

Embodiments of this invention also include transformation of R^{a}, R^{b}, R^{c}, R^{d}, and/or R^{e} of the starting materials of formulas (III) or (IV) to different R^{a}, R^{b}, R^{c}, R^{d}, and/or R^{e} of the products, which may take place during the reaction of the present invention or under the reaction conditions.

The present invention also provide a method (Scheme 3, Process III) for preparing a fluoroalkyl arylsulfinyl compound having a formula (I), which comprises reacting an oxygen-containing compound having a formula (II) with arylsulfur halotetrafluoride having a formula (IV) in the presence of a reducing substance that reduces the arylsulfur halotetrafluoride:

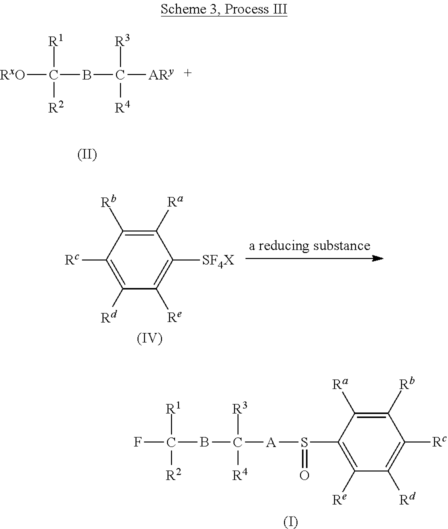

### Process III

Oxygen-containing compounds of formula (II) used for Process III are the same for Process I. Arylsulfur halotetrafluorides of formula (IV) used for Process III are the same as for Process II. Reducing substances used for Process III are the same as for Process II. Arylsulfur halotetrafluoride of the formula (IV) may be derived by an existing reducing substance to another compound(s) that can more effectively fluorinate an oxygen-containing compound having a formula (II). The compound(s) include any derived compounds that can fluorinate an oxygen-containing compound having a formula (II) to form the product (formula I). A preferrable derived compound is an arylsulfur trifluoride represented by the formula (III).

The reaction of an oxygen-containing compound of formula (II) with the arylsulfur halotetrafluoride in the presence of a reducing substance, wherein at least one of R^{x} and R^{y} is a hydrogen atom or a silyl group, or R^{x} and R^{y} combine to form a silyl group, may be conducted furthermore in the presence of a base or a silicon atom-activating agent. The base or silicon atom-activating agent is exemplified by the same as for Process I. The amount of the base or silicon atom-activating agent may be a catalytic amount to an amount in excess for the reaction of this invention, dependent on reaction conditions.

The reaction of an oxygen-containing compound of formula (II) with the arylsulfur halotetrafluoride in the presence of a reducing substance, wherein A is an oxygen atom, may also be conducted furthermore in the presence of hydrogen fluoride, a mixture of hydrogen fluoride and an amine(s), or a tetraalkylammonium fluoride-hydrogen fluoride. The hydrogen fluoride may be in situ generated in the same manner as described for Process I. The mixture of hydrogen fluoride and an amine(s), or the tetraalkylammonium fluoride-hydrogen fluoride, is exemplified as in Process I. The amount of the hydrogen fluoride, a mixture of hydrogen fluoride and an amine(s), or tetraalkylammonium fluoride-hydrogen fluoride, may be a catalytic amount to an amount in excess for the reaction of this invention, dependent on reaction conditions.

Process III can be carried out in the presence of one or more solvent(s) or in the absence of solvent. The use of solvent is preferable for mild and efficient reactions. When solvent is used, the preferable solvents will not substantially react with the starting materials and reagents, the intermediates, and/or the final product(s). Suitable solvents include the same solvents as were described in Process I above.

In order to obtain good yield of product in Process III, the reaction temperature can preferably be selected in the range of about −100° C.˜+200° C.; more preferably, the reaction temperature can be selected in the range of about −80° C.˜+150° C.; and furthermore preferably, the reaction temperature can be selected in the range of about −80° C.˜+120° C.

Reaction conditions of Process III are optimized to obtain economically good yield of product. In one illustrative embodiment, from about 0.5 mol to about 2 mol, more preferably, from about 0.8 mol to about 1.5 mol, furthermore preferably, from about 0.9 mol to about 1.2 mol of arylsulfur halotetrafluoride (formula IV) are combined with 1 mol of oxygen-containing compound (formula II) to obtain a good yield of fluoroalkyl arylsulfinyl compound (formula I).

The amount of a reducing substance greatly varies on the nature and reactivity of the reducing substance used. However, in one illustrative embodiment, from about 0.1 mol to about 5 mol, more preferably, from about 0.15 mol to 3 mol of a reducing substance can be selected against 1 mol of arylsulfur halotetrafluoride (formula IV) to obtain a good yield of the product (formula I).

Note that the reaction time for Process III varies dependent upon reaction temperature, and the types and amounts of substrates, reagents, and solvents. As such, reaction time is generally determined as the amount of time required to complete a particular reaction, but can be from about 1 minute to several days, preferably, within a few days.

R¹, R², R³, R⁴, R⁵, R⁶, R⁷, R⁸, R⁹, R¹⁰, R¹¹, R¹² and/or R¹³ of the products represented by the formula (I) may be different from the R¹, R², R³, R⁴, R⁵, R⁶, R⁷, R⁸, R⁹, R¹⁰, R¹¹, R¹² and/or R¹³ of the starting materials represented by the formula (II). Thus, embodiments of this invention include transformation of the R¹, R², R³, R⁴, R⁵, R⁶, R⁷, R⁸, R⁹, R¹⁰, R¹¹, R¹² and/or R¹³ to different R¹, R², R³, R⁴, R⁵, R⁶, R⁷, R⁸, R⁹, R¹⁰, R¹¹, R¹² and/or R¹³ which may take place during the reaction of the present invention or under the reaction conditions as long as both the fluorination and the arylsulfinylation take place.

Embodiments of this invention also include transformation of R^{a}, R^{b}, R^{c}, R^{d}, and/or R^{e} of the starting materials of formula (IV) to different R^{a}, R^{b}, R^{c}, R^{d}, and/or R^{e} of the products, which may take place during the reaction of the present invention or under the reaction conditions.

The fluoroalkyl arylsulfinyl compounds of formula (I) can be used as useful fluorinated compounds, intermediates, and building blocks (fluorinated synthons) for preparation of other useful fluorine-containing compounds (for example, see Examples 72˜83). Fluorine-containing compounds have wide application in medical, agricultural, and electronic materials as well as other like industries because the fluoro compounds show specific biological activity or physical properties based on the presence of one or more fluorine atoms [see Chemical & Engineering News, June 5, pp 13-22 (2006); Angew. Chem. Int. Ed., Vol. 39, pp 4216-4235 (2000)].

The fluoroalkyl arylsulfinyl compounds have an arylsulfinyl group, ArS(O)—, which is an excellent protecting group of a hydroxy or an amino group in organic synthesis. The arylsulfinyl group can easily be deprotected under mild conditions without altering other functional groups in a molecule (for example, see Examples 82 and 83). Accordingly, the arylsulfinyl compounds produce useful fluorine-containing alcohols having formula (V) or amines having formula (VI) as shown in Scheme 4 (which shows examples of the fluoroalkyl arylsulfinyl compounds of formula I).

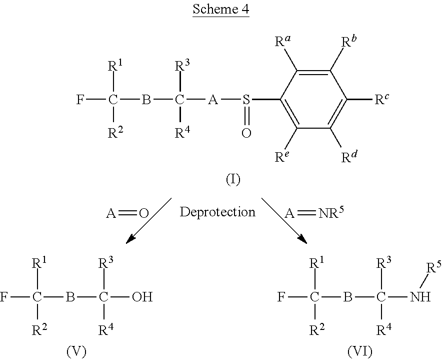

In Formulas (I), (V), and (VI), R¹, R², R³, R⁴, A, B, R⁵, R^{a}, R^{b}, R^{c}, R^{d}, and R^{e} are the same as defined above.

In particular, 3-fluoropyrrolidine or its salt as shown in Example 83, is an important fluorine-containing compound [see, Bioorganic & Medicial Chemistry Letters, Vol. 15, pp. 4770-4773 (2005) and Vol. 14, pp. 1265-1268 (2004); Synlett, 1995, pp. 55-57]. The present invention shows a surprisingly useful preparation of 3-fluoropyrrolidine (or its salt) from 3-pyrrolidinol via the compound and preparative methods of the present invention (see Examples 44, 45, and 83). A conventional preparative method of 3-fluoropyrrolidine is that 3-pyrrolidinol includes conversion with benzyl halide to N-benzylpyrrolidinol, which is then reacted with p-toluenesulfonyl chloride to give N-benzyl-3-(p-toluenesulfonyloxy)pyrrolidine, and treated with spray-dried potassium fluoride to give N-benzyl-3-fluoropyrrolidine, which is then deprotected by hydrogenation at 45 psi in the presence of 10% Pd/C catalyst to produce 3-fluoropyrrolidine or its salt (see Synlett, 1995, pp. 55-57). However, the conventional method for 3-fluoropyrrolidine is a multi-step method and provides a more costly product. As such, embodiments of the present invention provide significantly improved methodologies for preparation of, for example, 3-fluoropyrrolidine.

Furthermore, the arylsulfinyl group can easily be transformed to another functional group such as arylsulfonyl group, ArSO₂—. As shown in Scheme 5 and Examples 72˜79, a fluoroalkyl arylsulfinyl compounds having a formula (I) is converted to a fluoroalkyl arenesulfonate having a formula (VII) or fluoroalkyl arenesulfonamide having a formula (VIII), which are useful fluorinated intermediates and building blocks (synthons) for the preparation of useful fluorine-containing compounds. For example, a fluoroalkyl arenesulfonate is typically transformed to another fluoroalkyl derivative (see Examples 80 and 81).

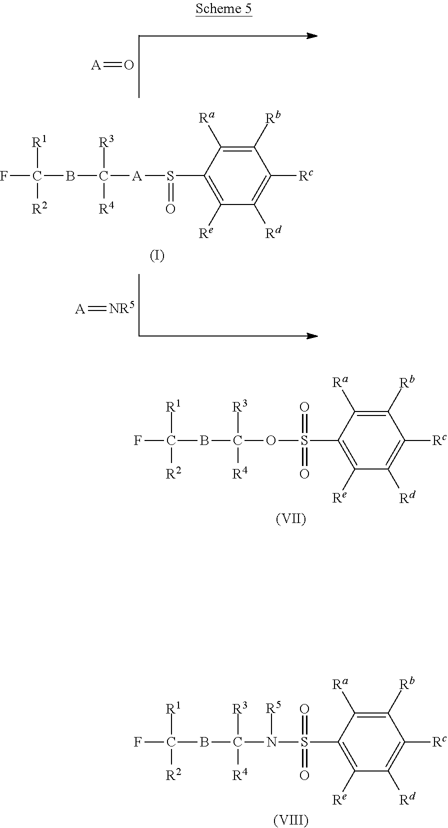

In Formulas (I), (VII), and (VIII), R¹, R², R³, R⁴, R⁵, A, B, R^{a}, R^{b}, R^{c}, R^{d}, and R^{e} are the same as defined above.

The compounds of the invention may comprise one or more chiral centers so that the compounds may exist as stereoisomers, including diasteroisomers, enantiomers, and rotamers (rotational isomers). All such compounds are within the scope of the present invention, including all such stereoisomers, and mixtures thereof, including racemates.

Another embodiment of the present invention provides novel useful fluoroalkyl arylsulfinyl compounds represented by formula (Ia):

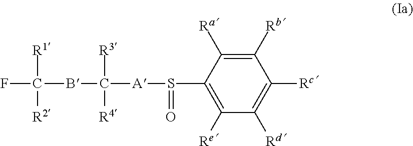

in which:
R^{1′}, R^{2′}, R^{3′}, and R^{4′} each is independently a hydrogen atom, a substituted or unsubstituted alkyl group having 1 to 20 carbon atoms, preferably 1 to 14 carbon atoms, a substituted or unsubstituted alkenyl group having 2 to 20 carbon atoms, preferably 2 to 14 carbon atoms, a substituted or unsubstituted aryl group having 6 to 20 carbon atoms, preferably 6 to 14 carbon atoms, a substituted or unsubstituted alkoxycarbonyl group having 2 to 20 carbon atoms, preferably 2 to 14 carbon atoms, a substituted or unsubstituted aryloxycarbonyl group having 7 to 20 carbon atoms, preferably 7 to 14 carbon atoms, or a cyano group;
R^{a′}, R^{b′}, R^{c′}, R^{d′}, and R^{e′} each is independently a hydrogen atom, a halogen atom, a substituted or unsubstituted alkyl group having 1 to 10 carbon atoms, preferably 1 to 4 carbon atoms, or a nitro group;
A′ is an oxygen atom or NR^{5′} in which R^{5′} is a hydrogen atom, a substituted or unsubstituted alkyl group having 1 to 20 carbon atoms, preferably 1 to 14 carbon atoms, a substituted or unsubstituted aryl group having 6 to 20 carbon atoms, preferably 6 to 14 carbon atoms;
B′ is

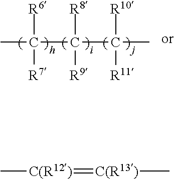

in which:
R^{6′}, R^{7′}, R^{8′}, R^{9′}, R^{10′}, R^{11′}, R^{12′} and R^{13′} each is independently a hydrogen atom, a halogen atom, a substituted or unsubstituted alkyl group having 1 to 20 carbon atoms, preferably 1 to 14 carbon atoms, a substituted or unsubstituted alkenyl group having 2 to 20 carbon atoms, preferably 2 to 14 carbon atoms, a substituted or unsubstituted aryl group having 6 to 20 carbon atoms, preferably 6 to 14 carbon atoms, a substituted or unsubstituted alkoxy group having 1 to 20 carbon atoms, preferably 1 to 14 carbon atoms, a substituted or unsubstituted aryloxy group having 6 to 20 carbon atoms, preferably 6 to 14 carbon atoms, a substituted or unsubstituted alkoxycarbonyl group having 2 to 20 carbon atoms, preferably 2 to 14 carbon atoms, a substituted or unsubstituted aryloxycarbonyl group having 7 to 20 carbon atoms, preferably 7 to 14 carbon atoms, a nitro group, or a cyano group;
h, i, and j each is independently 0 or 1; and
two or more of R^{1′}, R^{2′}, R^{3′}, R^{4′}, R^{5′}, R^{6′}, R^{7′}, R^{8′}, R^{9′}, R^{10′}, and R^{11′} or two or more of R^{1′}, R^{2′}, R^{3′}, R^{4′}, R^{5′}, R^{12′}, and R^{13′} may be linked with or without a heteroatom(s) to form any ring structure.

A preferable total number of h, i, and j of formula (Ia) is 2 or less, more preferably, 1 or 0, as based on availability and cost considerations.

The present invention also provides a method (Scheme 6; Process I′) for preparing a fluoroalkyl arylsulfinyl compound having a formula (Ia), which comprises reacting an oxygen-containing compound having a formula (IIa) with an arylsulfur trifluoride having a formula (IIIa):

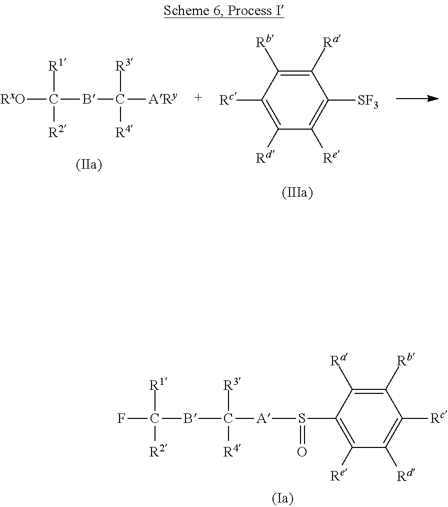

For the compounds represented by formulas (Ia), (IIa), and (IIIa), R^{1′}, R^{2′}, R^{3′}, R^{4′}, R^{x}, R^{y}, A′, B′, R^{a′}, R^{b′}, R^{c′}, R^{d′}, and R^{e′} are the same as described above.

### Process I′

Process I′ is the same as Process I except that compounds (IIa) and (IIIa) are used instead of compounds (II) and (III), respectively. Illustrative compounds of formulas (IIa) and (IIIa) are exemplified in Process I above.

The present invention also includes a method wherein the arylsulfur trifluoride of formula (IIIa) used for the Process 1′ (Scheme 6) is prepared by the method comprising reacting arylsulfur halotetrafluoride of formula (IVa) with a reducing substance. Thus, embodiments of the present invention provide a method (Scheme 7, Processes II′ and Ia′) for preparing a fluoroalkyl arylsulfinyl compound having a formula (Ia), which comprises (Process II′) reacting an arylsulfur halotetrafluoride having a formula (IVa) with a reducing substance that reduces the arylsulfur halotetrafluoride and (Process Ia′) reacting a resulting arylsulfur trifluoride having a formula (IIIa) with an oxygen-containing compound having a formula (IIa):

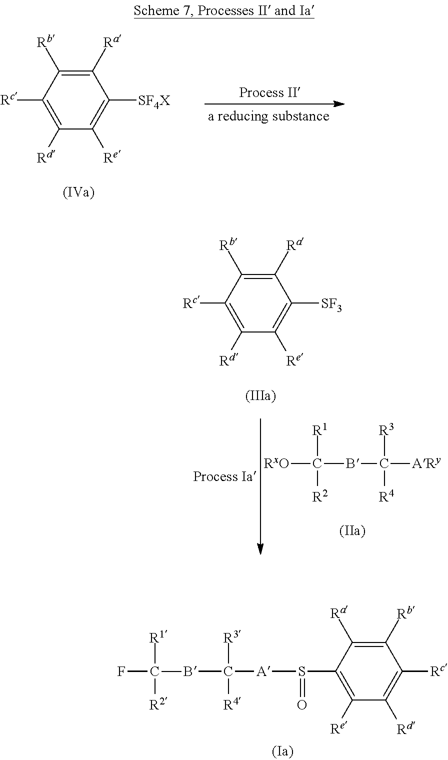

For the compounds represented by formulas (Ia), (IIa), (IIIa), and (IVa), R^{1′}, R^{2′}, R^{3′}, R^{4′}, R^{x}, R^{y}, A′, B′, R^{a′}, R^{b′}, R^{c′}, R^{d′}, R^{e′}, and X are the same as described above.

### Process II′

Process II′ is the same as Process II except that compound (IVa) is used instead of compound (IV). Illustrative compounds of formula (IVa) are exemplified in Process II above.

### Process Ia′

Process Ia′ is the same as Process Ia except that compounds (IIa) and (IIIa) are used instead of compounds (II) and (III), respectively. Illustrative compounds of formula (IIa) are exemplified in Process I above.

The present invention also provides a method (Scheme 8, Process III′) for preparing a fluoroalkyl arylsulfinyl compound having a formula (Ia), which comprises reacting an oxygen-containing compound having a formula (IIa) with arylsulfur halotetrafluoride having a formula (IVa) in the presence of a reducing substance that reduces the arylsulfur halotetrafluoride:

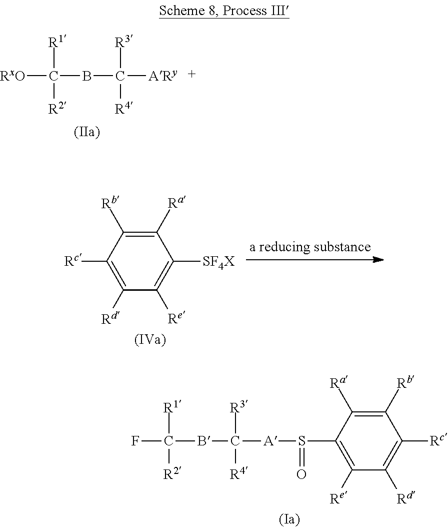

For the compounds represented by formulas (Ia), (IIa), and (IVa), R^{1′}, R^{2′}, R^{3′}, R^{4′}, R^{x}, R^{y}, A′, B′, R^{a′}, R^{b′}, R^{c′}, R^{d′}, R^{e′}, and X are the same as described above.

### Process III′

Process III′ is the same as Process III except that compounds (IIa) and (IVa) are used instead of compounds (II) and (IV), respectively. Illustrative compounds of formulas (IIa) and (IVa) are exemplified in Processes I and II above, respectively.

Embodiments of the present invention also provide useful fluoroalkyl arylsulfinyl compounds represented by formula (Ib) as follows:

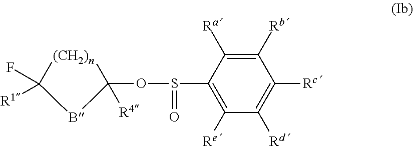

in which:

B″ is:

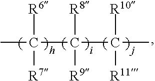

R^{1″} and R^{4″} each is independently a hydrogen atom, a substituted or unsubstituted alkyl group having 1 to 20 carbon atoms, preferably 1 to 14 carbon atoms, a substituted or unsubstituted aryl group having 6 to 20 carbon atoms, preferably 6 to 14 carbon atoms, a substituted or unsubstituted alkoxycarbonyl group having 2 to 20 carbon atoms, preferably 2 to 14 carbon atoms, a substituted or unsubstituted aryloxycarbonyl group having 7 to 20 carbon atoms, preferably 7 to 14 carbon atoms, or a cyano group;
R^{6″}, R^{7″}, R^{8″}, R^{9″}, R^{10″} and R^{11″} each is independently a hydrogen atom, a substituted or unsubstituted alkyl group having 1 to 20 carbon atoms, preferably 1 to 14 carbon atoms, or a substituted or unsubstituted aryl group having 6 to 20 carbon atoms, preferably 6 to 14 carbon atoms;
h, i and j each is independently 0 or 1;
n is 1, 2, 3, or 4; and
R^{a′}, R^{b′}, R^{c′}, R^{d′}, and R^{e′} are the same as described above.

Preferable total number of h, i, and j of formula (Ib) is 2 or less, more preferably 1 or 0, most preferably 0, as based on availability.

The fluoroalkyl arylsulfinyl compound of formula (Ib) can be prepared according to Process I′, Processes II′ and Ia′, or Process III′.

Embodiments of the present invention provide useful fluoroalkyl arylsulfinyl compounds represented by formula (Ic) as follows:

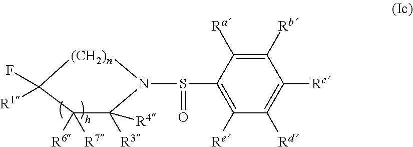

in which:
R^{1″}, R^{3″} and R^{4″} each is independently a hydrogen atom, a substituted or unsubstituted alkyl group having 1 to 20 carbon atoms, preferably 1 to 14 carbon atoms, a substituted or unsubstituted aryl group having 6 to 20 carbon atoms, preferably 6 to 14 carbon atoms, a substituted or unsubstituted alkoxycarbonyl group having 2 to 20 carbon atoms, preferably 2 to 14 carbon atoms, a substituted or unsubstituted aryloxycarbonyl group having 7 to 20 carbon atoms, preferably 7 to 14 carbon atoms, or a cyano group;
R^{6″} and R^{7″} each is independently a hydrogen atom, a substituted or unsubstituted alkyl group having 1 to 20 carbon atoms, preferably 1 to 14 carbon atoms, a substituted or unsubstituted aryl group having 6 to 20 carbon atoms, preferably 6 to 14 carbon atoms;
h is 0 or 1;
n is 1, 2, 3, or 4; and
R^{a′}, R^{b′}, R^{c′}, R^{d′}, and R^{e′} are the same as described above.

The fluoroalkyl arylsulfinyl compound of formula (Ic) can be prepared according to Process I′, Processes II′ and Ia′, or Process III′.

Embodiments of the present invention provide a fluoroalkyl arylsulfinyl compound having a formula (Id) as follows:

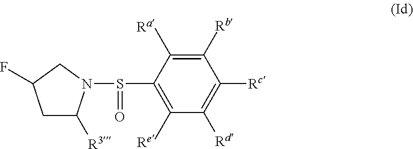

in which:
R^{3′″} is a hydrogen atom, a substituted or unsubstituted alkoxycarbonyl group having 2 to 20 carbon atoms, preferably 2 to 14 carbon atoms, a substituted or unsubstituted aryloxycarbonyl group having 7 to 20 carbon atoms, preferably 7 to 14 carbon atoms, or a cyano group; and
R^{a′}, R^{b′}, R^{c′}, R^{d′}, and R^{e′} are the same as described above.

The fluoroalkyl arylsulfinyl compound of formula (Id) can be prepared according to Process I′, Processes II′ and Ia′, or Process III′.

The fluoroalkyl arylsulfinyl compound (formula (I)) of the present invention is also prepared by reaction of a fluorine-containing compound having a formula (IX) with an arylsulfinyl fluoride having a formula (X) in the presence of a base, as shown in Scheme 9 (Process IV) (also, see Examples 70 and 71).

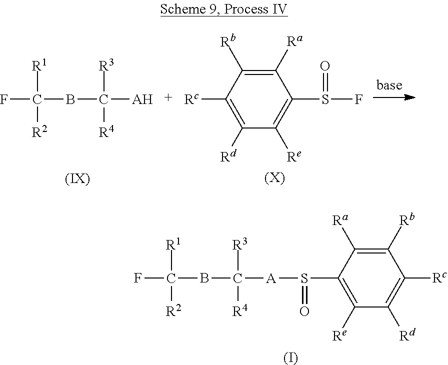

For the compounds represented by formulas (I), (IX), and (X), R¹, R², R³, R⁴, A, B, R^{a}, R^{b}, R^{c}, R^{d}, and R^{e} are the same as described above.

The arylsulfinyl fluoride (formula (X)) is prepared in a high yield from arylsulfur trifluoride (formula (III)) by reaction with a carboxylic acid, as shown in Example 69.

A fluoroalkyl arylsulfinyl compound having a formula (Ie) can also be prepared by Process V (Scheme 10) (also, see Example 31). Thus, the present invention provides a method (Scheme 10, Process V) for preparing a fluoroalkyl arylsulfinyl compound having a formula (Ie), which comprises reacting an oxygen-containing compound having a formula (IIb) with arylsulfur trifluoride having a formula (III):

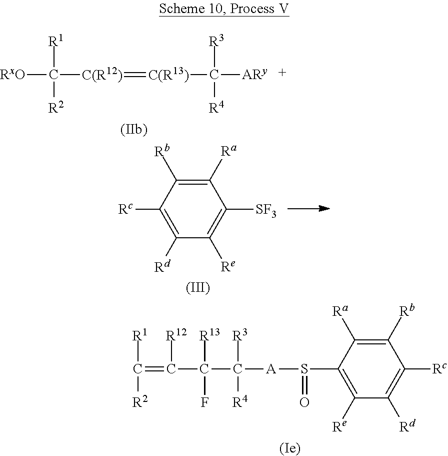

Compound (IIb) is compound (II) wherein the B is —C(R¹²)═C(R¹³)—.

For the compounds represented by formulas (IIb), (III), and (Ie), R¹, R², R³, R⁴, R¹², R¹³, A, R^{x}, R^{y}, R^{a}, R^{b}, R^{c}, R^{d}, and R^{e} are the same as described above.

### Process V

Process V is the same as Process I except that compound (IIb) is substituted for compound (II). Illustrative compounds of formula (IIb) are exemplifed in Process I above.

The present invention includes a method wherein the arylsulfur trifluoride of formula (III) used in Process V is prepared by the method comprising reacting arylsulfur halotetrafluoride of formula (IV) with a reducing substance. These processes are the same as Process II and Process Ia except that compound (IIb) is used instead of compound (II).

The present invention also provides a method for preparation of a compound of formula (Ie), which comprises reacting a compound of formula (IIb) with arylsulfur halotetrafluoride of formula (IV) in the presence of a reducing substance that reduces the arylsulfur halotetrafluoride. This process is the same as Process III except that compound (IIb) is used instead of compound (II).

A fluoroalkyl arylsulfinyl compound having a formula (If) can also be prepared by Process VI (scheme 11) (also, see Example 22). Thus, the present invention provide a method (Scheme 11, Process VI) for preparing a fluoroalkyl arylsulfinyl compound having a formula (If), which comprises reacting an oxygen-containing compound having a formula (IIc) with arylsulfur trifluoride having a formula (III):

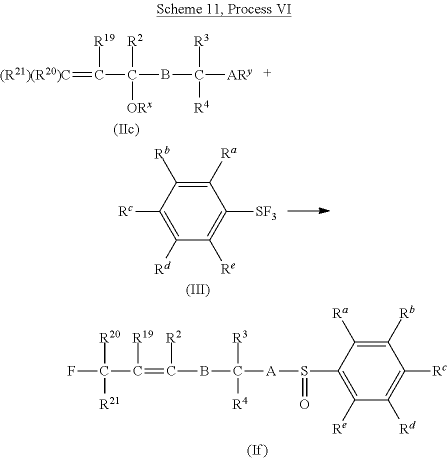

Compound (IIc) is the same as compound (II) wherein R¹ is —C(R¹⁹)═C(R²⁰)(R²¹).

For the compounds represented by formulas (IIc), (III), and (If), R², R³, R⁴, A, B, R^{x}, R^{y}, R^{a}, R^{b}, R^{c}, R^{d}, and R^{e} are the same as described above. R¹⁹, R²⁰, and R²¹ each is the same as defined for R⁶⁻¹³ described above.

### Process VI

Process VI is the same as Process I except that compound (IIc) is substituted for compound (II). Illustrative compounds of formula (IIc) are exemplified in Process I above.

The present invention includes a method wherein the arylsulfur trifluoride of formula (III) used in Process VI is prepared by the method comprising reacting arylsulfur halotetrafluoride of formula (IV) with a reducing substance. These processes are the same as Process II and Process Ia except that compound (IIc) is used instead of compound (II).

The present invention also provides a method for preparation of a compound of formula (If), which comprises reacting a compound of formula (IIc) with arylsulfur halotetrafluoride of formula (IV) in the presence of a reducing substance that reduces the arylsulfur halotetrafluoride. This process is the same as Process III except that compound (IIc) is used instead of compound (II).

The present invention provides preparative methods for fluoroalkyl arylsulfinyl compounds having a formula (I) and in addition, provides novel fluoroalkyl arylsulfinyl compounds having a formula (Ia), (Ib), (Ic) and/or (Id). The present invention also provides preparative methods for fluoroalkyl arylsulfinyl compounds having a formula (Ie) and/or (If). The preparative methods herein provide surprisingly good results, timely and convenient processes, improved safety, enhanced yields, and lower costs as compared to other conventional methods. The fluoroalkyl arylsulfinyl compounds are useful fluorinated compounds, fluoro intermediate compounds, or fluorinated building blocks, i.e., useful for the preparation of a desired fluoro compound.

The following examples will illustrate the present invention in more details, but it should be understood that the present invention is not deemed to be limited thereto.

### EXAMPLES

The following examples are provides for illustrative purposes only and are not intended to limit the scope of the invention.

### Example 1

### Preparation of 2-fluoroethyl benzenesulfinate (I-1)

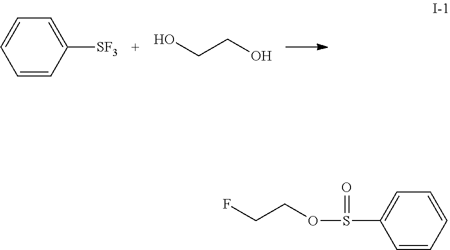

Phenylsulfur trifluoride (414 mg, 2.49 mmol) was taken in a vessel made of fluoropolymer (PFA) and dissolved in 2.5 mL of dry dichloromethane. The solution was cooled to −78° C., and into the solution was slowly added a solution of 155 mg (2.49 mmol) of ethylene glycol in 2.5 mL of dry dichloromethane during about 20 minutes. The reaction mixture was allowed to warm to room temperature and stirred at room temperature for 3 hours. An aqueous sodium carbonate solution was added to the reaction mixture. The organic layer was separated, dried over anhydrous magnesium sulfate, and filtered. Removal of solvent at reduced pressure gave 2-fluoroethyl benzenesulfinate (I-1), which was further purified by chromatography on silica gel: Yield 80%; ¹H-NMR (CDCl₃) δ 3.5-3.9 (m, 1H), 4.05-4.30 (m, 1H), 4.3-4.65 (dm, 2H, J=50 Hz), 7.4-7.55 (m, 3H), 7.6-7.7 (m, 2H); ¹⁹F-NMR (CDCl₃) δ −224.33 (m, 1F); ¹³C-NMR (CDCl₃) 63.03 (d, J=20 Hz), 81.86 (d, J=172 Hz), 125.34, 129.24, 132.51, 144.22.

### Example 2˜12

### Preparation of 2-fluoroethyl benzenesulfinate derivatives (I-1)˜(I-7)

Fluoroethyl benzenesulfinate derivates (I-1)˜(I-7) were prepared by reaction of arylsulfur trifluoride (III) with ethylene glycol, its salts or silyl derivatives in the same manner as described in Example 1. In Example 3, the reaction was carried out in the presence of a base (triethylamine). The results and reaction conditions are shown in Table 1 together with those of Example 1. When a silyl derivative was used as in Examples 5˜12, a solution of catalytic amount (0.1 mmol) of tetrabutylammonium fluoride (a silicon atom-activating agent) (1.0 M) in tetrahydrofuran (THF) was added to a solution of a silyl derivative and arylsulfur trifluoride.

TABLE 1
Preparation of fluoroalkyl arenesulfinates (I-1)~(I-7)

| Ex. | (II) | (III) | Solvent | Conditions | (I) | Yield |
|---|---|---|---|---|---|---|
| 1 | HOCH₂CH₂OH 2.49 mmol | 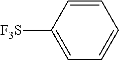 2.49 mmol | CH₂Cl₂ 5 mL | −78° C. −> r.t. 3 h | 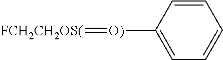 I-1 | 80% |
| 2 | HOCH₂CH₂OH 3.0 mmol | 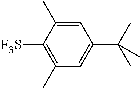 3.0 mmol | CH₂Cl₂ 5 mL | r.t. 3 h | 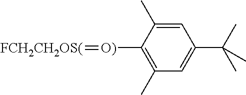 I-2 | 72% |
| 3 | HOCH₂CH₂OH 3.96 mmol | 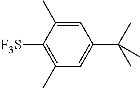 3.96 mmol | CH₂Cl₂ 4 mL | 0° C. −> r.t. 1 h r.t., 15 h Et₃N 7.72 mmol | 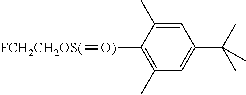 I-2 | 91% |
| 4 | NaOCH₂CH₂ONa 11.9 mmol | 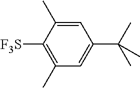 11.9 mmol | Tetrahydrofuran 30 mL | −78° C. −> r.t. 1 h r.t., 15 h | 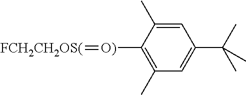 I-2 | 53% |
| 5 | Me₃SiOCH₂CH₂OSiMe₃ 2.98 mmol | 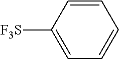 2.98 mmol | CH₂Cl₂ 4 mL | r.t. 2 h Bu₄NF (cat.) | 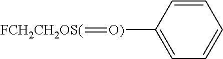 I-1 | 84% |
| 6 | Me₃SiOCH₂CH₂OSiMe₃ 2.29 mmol | 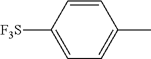 2.29 mmol | CH₂Cl₂ 8 mL | r.t. 2 h Bu₄NF (cat.) | 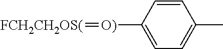 I-3 | 90% |
| 7 | Me₃SiOCH₂CH₂OSiMe₃ 3.80 mmol | 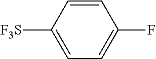 3.80 mmol | CH₂Cl₂ 8 mL | r.t. 2 h Bu₄NF (cat.) | 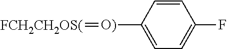 I-4 | 85% |
| 8 | Me₃SiOCH₂CH₂OSiMe₃ 4.86 mmol | 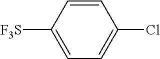 4.86 mmol | CH₂Cl₂ 8 mL | r.t. 2 h Bu₄NF (cat.) | 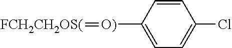 I-5 | 84% |
| 9 | Me₃SiOCH₂CH₂OSiMe₃ 4.80 mmol | 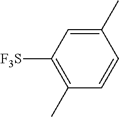 4.80 mmol | CH₂Cl₂ 8 mL | r.t. 2 h Bu₄NF (cat.) | 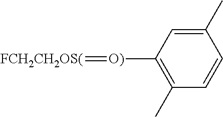 I-6 | 92% |
| 10 | Me₃SiOCH₂CH₂OSiMe₃ 5.80 mmol | 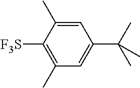 5.80 mmol | CH₂Cl₂ 10 mL | r.t. 2 h Bu₄NF (cat.) | 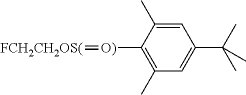 I-2 | 85% |
| 11 | Me₃SiOCH₂CH₂OSiMe₃ 3.70 mmol | 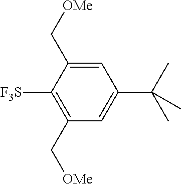 3.70 mmol | CH₂Cl₂ 8 mL | r.t. 2 h Bu₄NF (cat.) | 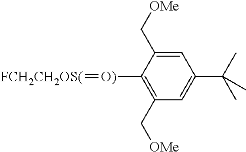 I-7 | 85% |
| 12 | 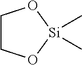 2.0 mmol | 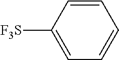 2.0 mmol | CH₂Cl₂ 5 mL | r.t. 3 h Bu₄NF (cat.) | 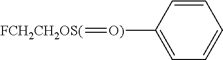 I-1 | 60% |

The properties and spectral data of the products are shown in the following:

2-fluoroethyl 4-tert-butyl-2,6-dimethylbenzenesulfinate (I-2); ¹H-NMR (CDCl₃) δ 1.28 (s, 9H), 2.62 (s, 6H), 4.17-4.42 (m, 2H), 4.64 (dt, 2H, J=47, 4 Hz), 7.04 (s, 2H); ¹⁹F-NMR (CDCl₃) δ −224.14 (m, 1F); ¹³C-NMR (CDCl₃) δ 19.49, 31.09, 34.74, 67.15 (d, J=20 Hz), 82.24 (d, J=172 Hz), 127.27, 137.60, 137.91, 155.26. Elemental analysis calculated for C₁₄H₂₁FO₂S: C, 61.97%; H, 7.77%. Found; C, 61.97%; H, 7.73%.

2-Fluoroethyl 4-methylbenzenesulfinate (I-3): ¹H-NMR (CDCl₃) δ 2.38 (s, 3H), 3.75 (dq, 1H), 4.18 (dq, 1H), 4.50 (dm, 2H, J=47.5 Hz), 7.35 (d, 2H, J=8.3 Hz), 7.56 (d, 2H, J=8.3 Hz); ¹⁹F-NMR (CDCl₃) δ −224.16 (tt, 1F, J=47.6, 28.1 Hz); ¹³C-NMR (CDCl₃): δ 21.58, 62.62 (d, J=20.2 Hz), 81.90 (d, J=171.9 Hz), 125.35, 129.89, 141.23, 143.21.

2-Fluoroethyl 4-fluorobenzenesulfinate (I-4): ¹H-NMR (CDCl₃) δ 3.74 (dq, 1H), 4.16 (dq, 1H), 4.46 (dm, 2H, J=47.0 Hz), 7.12 (m, 2H), 7.63 (m, 2H); ¹⁹F-NMR (CDCl₃) δ −106.35 (s, 1F), −224.18 (tt, 1F, J=47.6, 28.1 Hz); ¹³C-NMR (CDCl₃) δ 63.28 (d, J=20.2 Hz), 81.78 (d, J=171.2 Hz), 116.44 (d, J=22.4 Hz), 127.85 (d, J=9.4 Hz), 140.15 (d, J=2.8 Hz), 165.10 (d, J=252.9 Hz).

2-Fluoroethyl 4-Chlorobenzenesulfinate (I-5): ¹H-NMR (CDCl₃) δ 3.75 (dq, 1H), 4.17 (dq, 1H), 4.48 (dm, 2H, J=47.0 Hz), 7.42 (d, 2H, J=8.6 Hz), 7.58 (d, 2H, J=8.6 Hz); ¹⁹F-NMR (CDCl₃) δ −224.07 (tt, 1F, J=47.6, 28.1 Hz); ¹³C-NMR (CDCl₃) δ 63.40 (d, J=20.2 Hz), 81.76 (d, J=171 Hz), 126.88, 129.49, 138.78, 142.83.

2-Fluoroethyl 2,5-dimethylbenzenesulfinate (I-6): ¹H-NMR (CDCl₃) δ 3.73 (dq, 1H), 4.18 (dq, 1H), 4.50 (dm, 2H, J=47.1 Hz), 7.09 (d, 1H, J=6.9 Hz), 7.21 (d, 1H, J=6.9 Hz), 7.66 (s, 1H); ¹⁹F-NMR (CDCl₃) δ −223.87 (tt, 1F, J=47.0, 28.0 Hz); ¹³C-NMR (CDCl₃) δ 17.54, 21.04, 63.08 (d, J=21.0 Hz), 81.84 (d, J=171.9 Hz), 124.59, 131.40, 133.27, 133.72, 136.38, 140.94.

2-Fluoroethyl 2,6-bis(methoxymethyl)-4-tert-butylbenzenesulfinate (I-7): ¹H-NMR (CDCl₃) δ 1.32 (s, 9H), 3.39 (s, 6H), 4.15-4.40 (dm, 2H), 4.60 (dt, 2H, J=46.7 HZ), 4.84 (d, AB type, 4H), 4.89 (s, 2H); ¹⁹F-NMR (CDCl₃) (major isomer) δ −223.95 (tt, 1F, J=47.7, 28.1 Hz).

### Examples 13-33

### Preparation of Fluoroalkyl Arenesulfinates (I-8)˜(I-32)

Various fluoroalkyl arenesulfinates were prepared by reaction of arylsulfur trifluoride (III) with different diols and silyl derivates in the same manner as in Example 1. The reactions were conducted in the presence or absence of a base or a silicon atom-activating agent. The results and reaction conditions are shown in Table 2.

TABLE 2
Preparation of fluoroalkyl arenesulfinates (I-8)~(I-32)

| Ex. | (II) | (III) | Solvent | Conditions | (I) | Yield |
|---|---|---|---|---|---|---|
| 13 | HOCH₂CH(CH₃)OH 2.1 mmol | 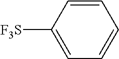 2.1 mmol | CH₂Cl₂ 5 mL | r.t. 3 h | 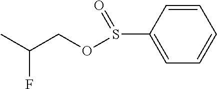 I-8 | 74% |
| 14 | HOCH₂CH(CH₃)OH 5.86 mmol | 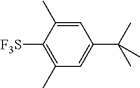 5.86 mmol | CH₂Cl₂ 10 mL | −78° C. −> r.t. 4 h pyridine (12 mmol) | 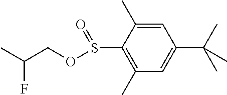 I-9 | 45% |
| 15 | HOCH₂CH(n-CH₁₄H₂₉)OH 2.0 mmol | 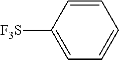 2.0 mmol | CH₂Cl₂ 5 mL | r.t. 24 h | 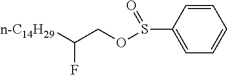 I-10 | 85% |
| 16 | HOCH₂CH(n-CH₁₄H₂₉)OH 2.0 mmol | 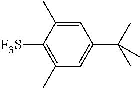 2.0 mmol | ClCH₂CH₂Cl 5 mL | reflux, 3 h | 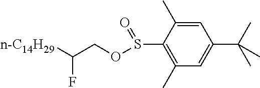 I-11 | 70% |
| 17 | 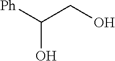 4.27 mmol | 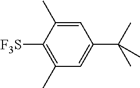 4.27 mmol | CH₂Cl₂ 10 mL | −78° C. −> r.t. 4 h pyridine (8.56 mmol) | 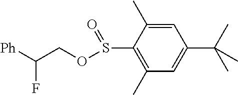 I-12 | 50% |
| 18 | 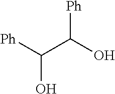 6.21 mmol | 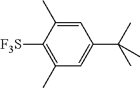 6.21 mmol | CH₂Cl₂ 10 mL | −78° C. −> r.t. 4 h pyridine (13.4 mmol) | 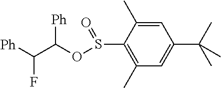 I-13 | 40% |
| 19 | ClCH₂CH(OH)CH₂OH 2.30 mmol | 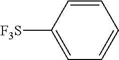 2.30 mmol | CH₂Cl₂ 5 mL | r.t. 3 h | 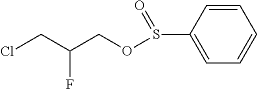 I-14 | 20% |
| | | | | | 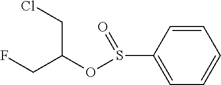 I-15 | 40% |
| 20 | CH₃OCH₂CH(OH)CH₂OH 2.30 mmol | 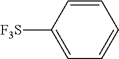 2.30 mmol | CH₂Cl₂ 5 mL | r.t. 3 h | 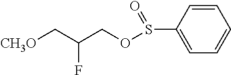 I-16 | 37% |
| | | | | | 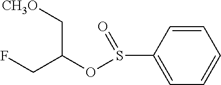 I-17 | 22% |
| 21 | PhOCH₂CH(OH)CH₂OH 2.45 mmol (Ph = phenyl) | 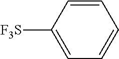 2.45 mmol | CH₂Cl₂ 5 mL | r.t. 3 h | 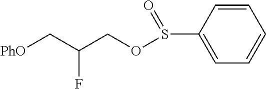 I-18 | 27% |
| | | | | | 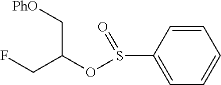 I-19 | 27% |
| 22 | CH₂═CHCH(OH)CH₂OH 2.85 mmol | 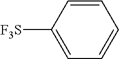 2.85 mmol | CH₂Cl₂ 5 mL | r.t. 3 h | 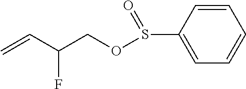 I-20 | 26% |
| | | | | | 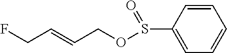 I-21 | 16% |
| 23 | CH₂═CH(CH₂)₃CH₂CH(OH)CH₂OH 2.85 mmol | 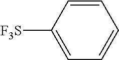 2.85 mmol | CH₂Cl₂ 5 mL | r.t. 3 h | 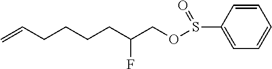 I-22 | 53% |
| 24 | HOCH₂CH₂CH₂OH 2.8 mmol | 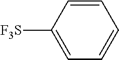 2.8 mmol | CH₂Cl₂ 5 mL | r.t., 3 h | 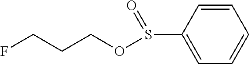 I-23 | 72% |
| 25 | HOCH₂CH₂CH₂OH 4.80 mmol | 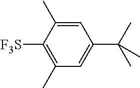 4.80 mmol | CH₂Cl₂ 5 mL | −78° C. −> r.t. 3 h | 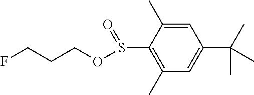 I-24 | 50% |
| 26 | 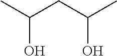 3.43 mmol | 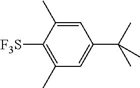 3.43 mmol | CH₂Cl₂ 10 mL | −78° C. −> r.t. 4 h pyridine (6.86 mmol) | 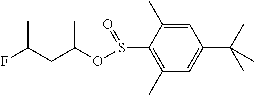 I-25 | 45% |
| 27 | 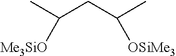 5.18 mmol | 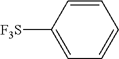 5.18 mmol | CH₂Cl₂ 8 mL | r.t., 2 h Bu₄NF (cat.) | 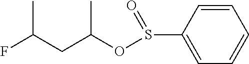 I-26 | 90% |
| 28 | 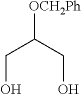 2.30 mmol | 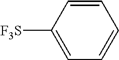 2.30 mmol | CH₂Cl₂ 5 mL | r.t., 4 h | 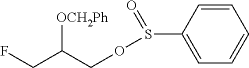 I-27 (Ph = phenyl) | 54% |
| 29 | 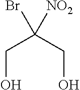 2.5 mmol | 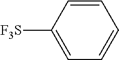 2.5 mmol | ClCH₂CH₂Cl 5 mL | reflux 3 h | 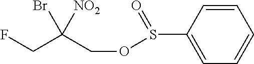 I-28 | 84% |
| 30 | 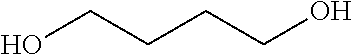 2.0 mmol | 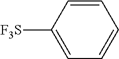 2.0 mmol | CH₂Cl₂ 5 mL | r.t., 24 h | 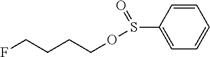 I-29 | 60% |
| 31 | 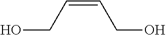 3.45 mmol | 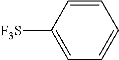 3.45 mmol | CH₂Cl₂ 5 mL | r.t., 4 h | 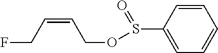 I-30 | 19% |
| | | | | | 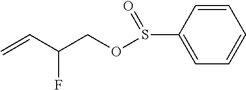 I-20 | 17% |
| 32 | 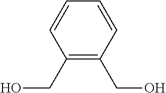 1.99 mmol | 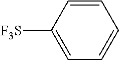 1.99 mmol | CH₂Cl₂ 5 mL | r.t., 4 h | 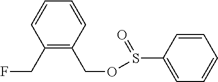 I-31 | 82% |
| 33 | 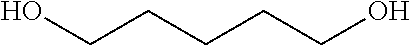 2.8 mmol | 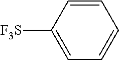 2.8 mmol | ClCH₂CH₂Cl 5 mL | reflux 3 h | 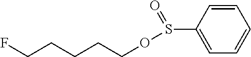 I-32 | 50% |

The properties and spectral data of the products (I-8)˜(I-32) are shown in the following:

2-Fluoropropyl benzenesulfinate (I-8): ¹H-NMR (CDCl₃) δ 1.2-1.42 (m, 3H), 3.4-4.20 (m, 2H), 4.6-5.0 (dm, 1H), 7.50-7.74 (m, 5H); ¹⁹F-NMR (CDCl₃) δ −180.45 (m, 1F).

2-Fluoropropyl 4-tert-butyl-2,6-dimethylbenzenesulfinate (I-9): ¹H-NMR (CDCl₃) δ 1.20-1.40 (overlapped singlet with multiplet, 12H), 2.62 (s, 6H), 3.50-4.25 (m, 2H), 4.65-5.05 (m, 1H), 7.03 (s, 2H); ¹⁹F-NMR (CDCl₃) δ −180.48 (m, 1F); ¹³C-NMR (CDCl₃) δ 17.21 (d, J=22 Hz), 19.48, 31.09, 34.72, 71.08 (d, J=22 Hz), 88.90 (d, J=17 Hz), 127.22, 137.52, 138.12, 155.15.

2-Fluorohexadecyl benzenesulfinate (I-10): ¹H-NMR (CDCl₃) δ 0.86 (t, J=6.5 Hz, 3H, CH₃), 1.15-1.70 (br.m, 26H, (CH₂)₁₃, 3.4-3.8 (m, 1H, CH), 3.95-4.25 (m, 1H, CH), 4.4-4.8 (d m, J=51 Hz, 1H, CHF), 7.45-7.60 (m, 3H, ArH), 7.20-7.80 (m, 2H, ArH); ¹⁹F-NMR (CDCl₃) δ −186.56 (m, 1F, CF); ¹³C-NMR (CDCl₃) δ 14.23, 22.78, 24.74, 24.90, 28.37, 29.47, 29.60, 29.75, 31.11, 31.38, 21.02, 65.60 (d, J=23 Hz), 91.8 (d, J=173 Hz), 125.37, 129.19, 132.42, 144.46

2-Fluorohexadecyl 4-tert-butyl-2,6-dimethylbenzenesulfinate (I-11): ¹H-NMR (CDCl₃) δ 0.88 (m, 3H, CH₃), 1.2-1.35 (m, 35H, 13×CH₂ & (CH₃)₃C), 2.63 (s, 6H, 2×CH₃), 4.02-4.26 (m, 2H, CH₂O), 4.67 (dm, J=47.8 Hz, CHF), 7.04 (s, 2H, ArH); ¹⁹F-NMR (CDCl₃) (a mixture of distereomers or rotamers) δ −186.99 (m, 0.75F), −186.36 (m, 0.25F); ¹³C-NMR (CDCl₃) (major stereoisomer) δ 14.23, 19.49, 22.80, 24.82, 29.43, 29.48, 29.51, 29.61, 29.76, 31.09, 32.03, 34.72, (d, J=22.3 Hz), 92.43 (d, J=173.2 Hz), 127.21, 137.59, 138.15, 155.13.

2-Fluoro-2-phenylethyl 4-tert-butyl-2,6-dimethylbenzenesulfinate (I-12): ¹H-NMR: (CDCl₃): δ 1.31 (s, 9H), 2.65 (s, 6H), 4.10-4.50 (m, 2H), 5.50-5.90 (m, 1H) 7.07 (s, 2H), 7.30-7.50 (m, 5H); ¹⁹F-NMR (CDCl₃) δ −183.86 (m, 1F); ¹³C-NMR (CDCl₃) δ 19.51, 31.12, 34.71, 21.31, 22.07, 44.93, (d, J=21 Hz), 73.23, 87.18 (d, J=165 Hz), 127.16, 136.98, 138.75, 154.81.

1,2-Diphenyl-2-fluoroethyl 4-tert-butyl-2,6-dimethylbenzenesulfinate (I-13): ¹H-NMR (CDCl₃) (as a mixture of two isomers of about 1:1 ratio) δ 1.29 and 1.31 (two singlet, 9H), 2.38 and 2.45 (two singlet, 6H), 5.40-5.55 (m, 1H), 5.75 (m, 1H), 6.9-7.4 (m, 12 H); ¹⁹F-NMR (CDCl₃) (as a mixture of two isomers of about 1:1 ratio): δ −180.45 (dd, 1F, J=44.8, 13.0 Hz) and δ −185.01 (dd, 1F, J=46.5, 16.2 Hz); ¹³C-NMR (CDCl₃) (a mixture of two isomers of about 1:1 ratio) δ 19.13, 19.33, 31.14, 34.76, 81.32 (d, J=26.7 Hz), 82.62 (d, J=28.2 Hz), 94.94 (d, J=180.9 Hz), 95.04 (d, J=180.6 Hz), 127.00, 127.08, 127.56, 127.62, 128.07, 128.32, 128.94, 135.12, 135.35, 135.63, 135.90, 137.44, 137.68, 138.06, 138.41, 139.44, 155.07, 155.22.

3-Chloro-2-fluoropropyl benzenesulfinate (I-14): ¹H-NMR (CDCl₃) δ 3.60-3.86 (m, 3H, CH₂Cl & CHO), 4.15-4.34 (m, 1H, CHO), 4.79 (dm, J=46.1 Hz, CHO), 7.57 (m, 3H, ArH), 7.73 (m, 2H, ArH); ¹⁹F-NMR (CDCl₃) δ −188.03 (m, CF); ¹³C-NMR (CDCl₃) δ 41.98 (d, J=25.3 Hz), 61.89 (d, J=24.5 Hz), 89.74 (d, J=180.5 Hz), 125.40, 129.36, 132.75, 143.87; GC-MS (m/e, intensity): 236 (M⁺, 5), 142 (PhSO₂+H, 35), 125 (M⁺-ClCH₂CHFCH₂O, 100), 77 (C₆H₅⁺. 80), 45 (MeOCH₂⁺, 100). Elemental analysis calculated for C₉H₁₀ClFO₃S: C, 45.67%; H, 4.26%. Found; C, 45.14%, H, 4.34%.

2-Fluoro-(1-chloromethyl)ethyl benzenesulfinate (I-15): ¹H-NMR (CDCl₃) δ 3.51 (m, 2H, CH₂Cl), 4.52-4.80 (m, 3H, CHO & CH₂F), 7.58 (m, 3H, ArH), 7.77 (m, 2H, ArH); ¹⁹F-NMR (CDCl₃) δ −231.93 (td, J=47.5 Hz, 15.6 Hz, CH₂F); ¹³C-NMR (CDCl₃) δ 41.57 (d, J=7.2 Hz), 74.72 (d, J=20.2 Hz), 82.11 (d, J=173.9 Hz), 125.21, 129.32, 132.93, 144.50; GC-MS (m/e, intensity) 236 (M⁺, 5), 142 (PhSO₂+H, 10), 125 (M⁺-ClCH₂CHFCH₂O, 100), 77 (C₆H₅⁺. 50).

2-Fluoro-3-methoxy-1-propyl benzenesulfinate (I-16): ¹H-NMR (CDCl₃) δ 3.38 (s, 3H, CH₃), 3.50-3.68 (m, 2H, CH₂O), 3.72-3.88 (m, 1H, CHOS), 4.15-4.30 (m, 1H, CHOS), 4.74 (dm, J=47.8 Hz, 1H, CHF), 7.57 (m, 3H, ArH), 7.74 (m, 2H, ArH); ¹⁹F-NMR (CDCl₃) δ −193.51 (d, quin, J=47.8, 22.4 Hz, CF); ¹³C-NMR (CDCl₃) δ 59.59, 62.99 (d, J=23.8 Hz), 71.25 (d, J=22.4 Hz), 90.32 (d, J=173.1 Hz), 125.38, 129.29, 132.56, 144.21; GC-MS (m/e, intensity): 232 (M⁺, 2), 125 (M⁺-MeOCH₂CHFCH₂O, 40), 91 (MeOCH₂CHFCH₂⁺, 75), 77 (C₆H₅⁺. 30), 45 (MeOCH₂⁺, 100). Elemental analysis calculated for C₁₀H₁₃FO₃S: C, 51.71%; H, 5.64%. Found; C, 51.58%, H, 5.64%.

2-Fluoro-(1-methoxymethyl)ethyl benzenesulfinate (I-17): ¹H-NMR (CDCl₃) δ 3.28 (s, 3H, CH₃), 3.40-3.49 (m, 2H, CH₂O), 4.40-4.78 (m, 3H, CH₂O & CHOS), 7.55 (m, 3H, ArH), 7.76 (m, 2H, ArH); ¹⁹F-NMR (CDCl₃) δ −230.55 (m, CH₂F); ¹³C-NMR (CDCl₃) δ 59.44, 70.91 (d, J=7.2 Hz), 75.49 (d, J=11.5 Hz), 82.86 (d, J=172.5 Hz), 125.26, 129.14, 132.53, 145.07; GC-MS (m/e, intensity): 232 (M⁺, 2), 125 (M⁺-MeOCH₂CHFCH₂O, 50), 91 (MeOCH₂CHFCH₂⁺, 90), 77 (C₆H₅⁺, 60), 45 (MeOCH₂⁺, 100).

2-Fluoro-3-phenoxy-propyl benzenesulfinate (I-18): ¹H-NMR (CDCl₃) δ 3.78-4.00 (m, 1H, CHOS), 4.08-4.24 (m, 2H, CH₂O), 4.24-4.44 (m, 1H, CHOS), 4.93 (dm, J=51.6 Hz, 1H, CHF), 6.88 (m, 2H, ArH), 6.99 (m, 1H, ArH), 7.29 (m, 2H, ArH), 7.56 (m, 3H, ArH), 7.73 (m, 2H, ArH); ¹⁹F-NMR (CDCl₃) δ −194.05 (d. quin, J=51.6, 20.7 Hz, CF); ¹³C-NMR (CDCl₃) δ 62.48 (d, J=24.5 Hz), 66.48 (d, J=24.5 Hz), 89.41 (d, J=177.5 Hz), 114.64, 121.61, 125.42, 129.32, 129.66, 132.64, 144.04, 158.14; GC-MS (m/e, intensity): 294 (M⁺, 5), 201 (M⁺-PhO, 10), 152 [(M⁺-(PhSO₂+H), 45], 125 (M⁺-PhOCH₂CHFCH₂O, 70), 107 (PhOCH₂⁺, 70), 77 (C₆H₅⁺, 100).

2-Fluoro-1-(phenoxymethyl)ethyl benzenesulfinate (I-19): ¹H-NMR (CDCl₃) δ 4.04 (m, 2H, CH₃O), 4.58-4.89 (m, 3H, CH₂F & CHOS), 6.77 (m, 2H, ArH), 6.96 (m, H, ArH), 7.25 (m, 2H, ArH), 7.58 (m, 2H, ArH), 7.79 (m, 2H, ArH); ¹⁹F-NMR (CDCl₃) 8-231.24 (dt, J=47.5 Hz, 19.0 Hz), CH₂F); ¹³C-NMR (CDCl₃) δ 65.84 (d, J=6.5 Hz), 74.32 (d, J=19.5 Hz), 82.74 (d, J=173.2 Hz), 114.54, 121.54, 125.38, 129.23, 129.63, 132.70, 144.80, 157.91; GC-MS (m/e, intensity): 294 (M⁺, 5), 201 (M⁺-PhO, 10), 152 [(M⁺-(PhSO₂+H), 45], 125 (M⁺-PhOCH₂CHFCH₂O, 70), 107 (PhOCH₂⁺, 70), 77 (C₆H₅⁺, 100).

2-Fluoro-3-butenyl benzenesulfinate (I-20): ¹H-NMR (CDCl₃) (8): 3.48-3.82 (m, 1H, CHO), 4.04-4.22 (m, 1H, CHO), 4.95-5.48 (m, 3H, CHF and CH₂═), 5.68-5.96 (m, 1H, CH═), 7.55 (m, 3H, ArH), 7.72 (m, 2H, ArH); ¹⁹F-NMR (CDCl₃) δ −185.48 (m, CHF).

4-Fluoro-2-trans-butenyl benzenesulfinate (I-21): ¹H-NMR (CDCl₃) δ 4.16 (m, 1H, CH═), 4.55 (m, 1H, CH═), 4.85 (dm, J=48 Hz, 2H, CH₂F), 5.86 (m, 2H, CH₂O), 7.56 (m, 3H, ArH), 7.73 (m, 2H, ArH); ¹⁹F-NMR (CDCl₃) δ −215.67 (tm, J=48 Hz. 1F, CHF); ¹³C-NMR (CDCl₃) δ 63.65, 82.18 (d, J=165.2 Hz), 125.35, 128.02 (d, J=11.5 Hz), 129.22, 129.45, 132.44, 144.48.

2-Fluoro-7-octenyl benzenesulfinate (I-22): ¹H-NMR (CDCl₃) δ 1.2-1.8 (m, 6H, (CH₂)₃), 2.02 (m, 2H, CH₂), 3.45-3.78 (m, 1H, ═CHH), 4.0-4.18 (m, 1H, ═CHU), 4.59 (br.d, J=50.5 Hz, 1H, CHF), 4.88-5.03 (m, 2H, CH₂O), 5.67-5.84 (m, 1H, CH═), 7.53 (m, 3H, ArH), 7.73 (m, 2H, ArH); ¹⁹F-NMR (CDCl₃) δ −186.76 (m, CF); ¹³C-NMR (CDCl₃) δ 24.23, 28.51, 31.21 (d, J=7.2 Hz), 33.51, 64.68, 64.98, 65.31, 65.61, 91.61 (d, J=173.3 Hz), 114.75, 125.32, 129.51, 132.41, 138.48, 144.40.

3-Fluoropropyl benzenesulfinate (I-23): ¹H-NMR (CDCl₃) δ 1.70-2.10 (m, 2H), 3.30-4.70 (m, 4H), 7.45-7.75 (m, 5H); ¹⁹F-NMR (CDCl₃) δ −222.65 (m, 1F); ¹³C-NMR (CDCl₃) δ 30.86 (d, J=19.5 Hz), 60.39 (d, J=5.05 Hz), 80.25 (d, J=165.4 Hz), 125.27, 129.20, 132.21, 136.36, 144.46.

3-Fluoropropyl 4-tert-butyl-2,6-dimethylbenzenesulfinate (I-24): m.p. 62-63° C.; ¹H-NMR (CDCl₃) δ 1.28 (s, 9H), 2.06-2.17 (dt, 2H, J=25.7, 5.9 Hz), 2.62 (s, 6H), 4.21 (t, 2H, J=6.2 Hz), 4.54 (dt, 2H, J=46.7, 5.8 Hz), 7.03 (s, 2H); ¹⁹F-NMR (CDCl₃) δ −222.69 (tt, 1F, J=58.5, 13 Hz); ¹³C-NMR (CDCl₃) δ 19.50, 31.10, 31.32 (d, J=20.2 Hz), 43.73, 64.65 (d, J=5.1 Hz), 80.29 (d, J=165.4 Hz), 127.23, 137.38, 138.19, 155.08. Elemental analysis calculated for C₁₅H₂₃FO₂S: C, 62.90%; H, 8.09%. Found; C, 63.18%; H, 8.36%.

3-Fluoro-1-methylbutyl 4-tert-butyl-2,6-dimethylbenzenesulfinate (I-25): ¹H-NMR (CDCl₃) δ 1.20-1.50 (singlet overlapped multiplets, 15H), 1.50-2.50 (m, 2H), 2.62 (s, 6H), 4.40-5.20 (m, 2H), 7.03 (m, 2H); ¹⁹F-NMR (CDCl₃) δ −175.08 (m, 1F); ¹³C-NMR (CDCl₃) δ 19.48, 31.10, 31.32 (d, J=20.2 Hz), 43.73, 64.65 (d, J=5.1 Hz), 80.29 (d, J=165.4 Hz), 127.23, 137.38, 138.19, 155.08. Elemental analysis calculated for C₁₇H₂₇FO₂S: C, 64.93%; H, 8.65%. Found; C, 64.50%; H, 8.80%.

3-Fluoro-1-methylbutyl benzenesulfinate (I-26): ¹H-NMR (CDCl₃) δ 0.50-2.0 (m, 8H), 4.2-4.80 (m, 2H), 7.30-7.80 (m, 5H); ¹⁹F-NMR (CDCl₃) (major isomer) δ −175.48 (dm, 1F);

3-Fluoro-2-(benzyloxy)propyl benzenesulfinate (I-27): ¹H-NMR (CDCl₃) δ 3.62-3.70 (m, 1H, CHOS), 3.80 (d.quint, J=18.9 Hz, 4.6 Hz, 1H, CHO), 4.08-4.16 (m, 1H, CHOS), 4.33-4.65 (m, 3H, CF₂F & CHOS), 7.30 (m, 5H, ArH), 7.51 (m, 3H, ArH), 7.69 (m, 2H, ArH); ¹⁹F-NMR (CDCl₃) δ −230.66 (t, d J=46.6 Hz, 19.0 Hz, CH₂F); ¹³C-NMR (CDCl₃) δ 61.71 (d, J=7.2 Hz), 72.38, 75.88 (d, J=20.2 Hz), 82.42 (d, J=171.1 Hz), 125.43, 127.89, 128.03, 128.58, 129.30, 132.56, 137.76, 144.18.

2-Bromo-3-fluoro-2-nitropropyl benzenesulfinate (I-28): ¹H-NMR (CDCl₃) δ 4.0-5.34 (m, 4H, CH₂), 7.5-7.8 (m, 5H, ArH); ¹⁹F-NMR (CDCl₃) δ −215.70 (m, 1F, CF); ¹³C-NMR (CDCl₃) δ 61.94, 81.12 (d, J=185.5 Hz), 87.52 (d, J=20.2 Hz), 125.31, 129.57, 133.30, 143.06.

4-Fluorobutyl benzenesulfinate (I-29): ¹H-NMR (CDCl₃) δ 1.74 [m, 4H, (CH₂)₂], 3.61 (m, 1H, CHO), 4.06 (m, 1H, CHO), 4.40 (dm, J=47 Hz, CF₂H), 7.52 (m, 3H, ArH), 7.68 (m, 2H, ArH); ¹⁹F-NMR (CDCl₃) δ: −218.98 (m, 1F, CH₂F); ¹³C-NMR (CDCl₃) δ 25.75 (d, J=4.3 Hz), 26.92 (d, J=20.2 Hz), 63.95, 83.44 (d, J=164.5 Hz), 125.27, 129.16, 132.26, 144.60; GC-MS (m/e, intensity) 216 (M⁺, 2), 142 (M+-FCH₂CH₂CH₂CH, 30), 125 (PhSO⁺, 50), 78 (C₆H₆⁺. 100), 55 (CH₂CH₂CH₂CH⁺, 80).

4-Fluoro-2-cis-butenyl benzenesulfinate (I-30): ¹H-NMR (CDCl₃) δ 4.2 (m, 1H, CH═), 4.58 (m, 1H, CH═), 4.87 (dd, J=47 Hz, 6.0 Hz, 2H, CH₂F), 5.75 (m, 2H, CF₂O), 7.57 (m, 3H, ArH), 7.73 (m, 2H, ArH); ¹⁹F-NMR (CDCl₃) δ −214.51 (t, d, J=47 Hz, 14 Hz, CH₂F); ¹³C-NMR (CDCl₃): δ: 59.30, 65.56 (d, J=23 Hz), 79.66, 91.11 (d, J=174.6 Hz), 125.38, 129.25, 132.55, 144.38.

[(o-Fluoromethyl)phenyl]methyl benzenesulfinate (I-31): ¹H-NMR (CDCl₃) δ 4.63 (d, J=11.7 Hz, 1H, CHO), 5.13 (d, J=11.7 Hz, 1H, CHO), 5.40 (dm, J=47.8 Hz, 2H, CH₂F), 7.35 (m, 4H, ArH), 7.56 (m, 3H, ArH), 7.74 (m, 2H, ArH); ¹⁹F-NMR (CDCl₃) δ −208.04 (t, J=47.8, CH₂F); ¹³C-NMR (CDCl₃) δ 62.95, 82.26 (d, J=165.3 Hz), 125.38, 129.02, 129.13, 129.22, 129.28, 129.36, 130.34, 132.53, 133.71, 135.14 (d, J=15.8 Hz), 144.33; GC-MS (m/e, intensity) 264 (M⁺, 2), 123 (PhSO₂⁺, 100), 77 (C₆H₅⁺, 10).

5-Fluoropentyl benzenesulfinate (I-32): ¹H-NMR (CDCl₃) δ 1.36-1.49 (m, 2H, CH₂), 1.54-1.73 (m, 4H, 2×CH₂), 3.58 (m, 1H, CHO), 4.02 (m, 1H, CHO), 4.38 (dt, J=47.4, 5.8 Hz, CH₂F), 7.52 (m, 3H, ArH), 7.68 (m, 2H, ArH); ¹⁹F-NMR (CDCl₃) δ −218.45 (tt, J=47.4, 25.2 Hz); ¹³C-NMR (CDCl₃) δ 21.68 (d, J=5 Hz), 29.32, 29.90 (d, J=19.5 Hz), 64.29, 83.84 (d, J=164.6 Hz), 125.29, 129.14, 132.20, 144.67; GC-MS (m/e, intensity) 230 (M⁺, 1), 143 (M⁺-F(CH₂)₄C, 100), 125 (PhSO⁺, 50), 78 (C₆H₆⁺, 70).

### Examples 34-36

### Preparation of a cyclic type of fluoroalkyl arenesulfinates (I-33)˜(I-35)

A cyclic type of fluoroalkyl arenesulfinate was prepared by reaction of arylsulfur trifluoride (III) with different cycloalkanediols or silyl derivatives in the same manner as in Example 1. The results and reaction conditions are shown in Table 3.

TABLE 3
Preparation of cyclic fluoroalkyl arenesulfinates (I-33)~(I-35)

| Ex. | (II) | (III) | Solvent | Conditions | (I) | Yield |
|---|---|---|---|---|---|---|
| 34 | 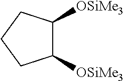 3.5 mmol | 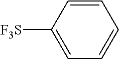 3.5 mmol | CH₂Cl₂ 5 mL | r.t., 3 h Bu₄NF (cat.) | 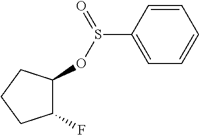 I-33 | 80% |
| 35 | 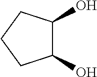 3.71 mmol | 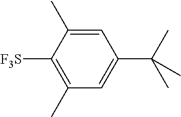 3.71 mmol | CH₂Cl₂ 10 mL | −78° C. −> r.t. 23 h pyridine (13.5 mmol) | 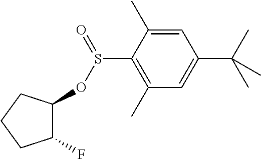 I-34 | 43% |
| 36 | 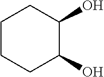 4.07 mmol | 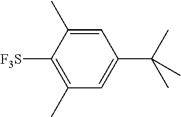 4.07 mmol | CH₂Cl₂ 10 mL | −78° C. −> r.t. 17 h pyridine (8.1 mmol) | 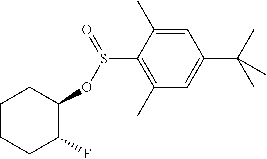 I-35 | 50% |

The properties and spectral data of the products (I-33)˜(I-35) are shown in the following:

1-Fluoro-2-(phenylsulfinyloxy)cyclopentane (I-33): ¹H-NMR (CDCl₃) δ 1.50-2.0 (m, 6H), 4.58-4.80 (m, 1H), 5.00 (dt, 1H, J=51.6, 2.4 Hz), 7.35-7.55 (m, 3H), 7.58-7.80 (m, 2H); ¹⁹F-NMR (CDCl₃) (major isomer) δ −179.68 (m, 1F); ¹³C-NMR (CDCl₃) δ 21.00, 30.25, 30.80, 81.36 (d. J=28.9 Hz), 98.32 (d. J=177.7 Hz), 125.80, 129.19, 132.44, 184.85.

1-Fluoro-2-(4-tert-butyl-2,6-dimethylphenylsulfinyloxy)cyclopentane (I-34): a mixture of two isomers (92:8); viscous liquid; spectral data of major isomer: ¹H-NMR (CDCl₃) δ 1.28 (s, 9H), 1.70-2.25 (m, 6H), 2.61 (s, 6H), 4.70-5.20 (m, 2H), 7.01 (s, 2H); ¹⁹F-NMR (CDCl₃) δ −179.7 (m, 1F); ¹³C-NMR (CDCl₃) δ 19.47, 21.11, 30.34 (d, J=21.0), 31.10, 34.73, 83.78 (d, J=27.5 Hz), 98.24 (d, J=178.4 Hz), 127.20, 137.26, 138.46, 155.09.

Elemental analysis calculated for C₁₇H₂₅FO₂S: C, 65.35%; H, 8.09%. Found; C, 64.99%; H, 8.19%.

1-Fluoro-2-(4-tert-butyl-2,6-dimethylphenylsulfinyloxy)cyclohexane (I-35): a mixture of two isomers (92:8): viscous liquid: Spectral data of major isomer: ¹H-NMR (CDCl₃) δ 1.20-1.33 (m, 11H), 1.35-2.38 (m, 6H), 2.62 (s, 6H), 4.20-4.27 (m, 2H), 7.02 (s, 2H); ¹⁹F-NMR (CDCl₃) δ −179.89 (br.d, 1F); ¹³C-NMR (CDCl₃) δ 19.42, 22.98 (d, J=10.1 Hz), 23.65, 30.80 (d, J=17.3 Hz), 31.12, −32.04, 34.70, 81.09 (d, J=17.3 Hz), 93.50 (d, J=179.2 Hz), 127.05, 137.22, 138.96.

### Examples 37-43

### Preparation of Fluoroalkyl Arenesulfinamide (I-36)˜(I-42)

General procedure for Examples 37˜42: An arylsulfur trifluoride was dissolved in dichloromethane in a vessel made of fluoropolymer (PFA) under nitrogen. A solution of a trimethylsilyl derivative of an amino alcohol in dichloromethane was slowly added, at room temperature. The mixture was stirred at room temperature for 1 hour, and poured on aqueous sodium carbonate solution and extracted with dichloromethane. The organic layer was dried over anhydrous magnesium sulfate and filtered. Removal of solvent at reduced pressure gave the desired product. The results and reaction conditions are shown in Table 4.

Procedure for Example 43; Both reactants were dissolved in equal volume of heptane (4 ml each). About 17 ml of heptane was taken in a fluoropolymer (PFA) vessel and heated to 100° C. Both reactants were taken in separate syringe connected with Teflon tube on the end. Both the Teflon tube ends were immersed in heptane in the PFA vessel. Addition was done simultaneously for 2 hours. After complete addition the reaction mixture was further stirred for 0.5 hour at 100° C. After the reaction, insoluble black resin was removed, and the solution was washed with aqueous sodium bicarbonate solution, dried on magnesium sulfate, and filtered. Removal of solvent at reduced pressure gave the product. The results and reaction conditions are shown in Table 4.

TABLE 4
Preparation of fluoroalkyl arenesulfinamides (I-36)~(I-42)

| Ex. | (II) | (III) | Solvent | Conditions | (I) | Yield |
|---|---|---|---|---|---|---|
| 37 | 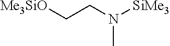 3.72 mmol | 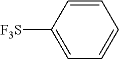 3.72 mmol | CH₂Cl₂ 5 mL | r.t., 1 h | 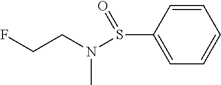 I-36 | 91% |
| 38 | 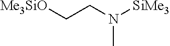 7.30 mmol | 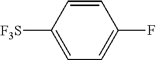 7.30 mmol | CH₂Cl₂ 5 mL | r.t., 1 h | 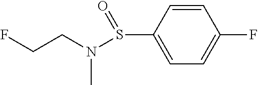 I-37 | 90% |
| 39 | 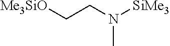 6.51 mmol | 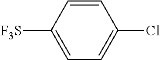 6.51 mmol | CH₂Cl₂ 5 mL | r.t., 1 h | 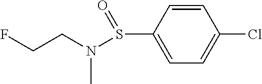 I-38 | 92% |
| 40 | 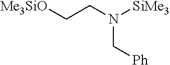 7.0 mmol | 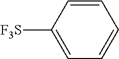 7.0 mmol | CH₂Cl₂ 5 mL | r.t., 1 h | 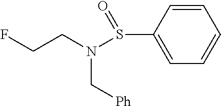 I-39 | 92% |
| 41 | 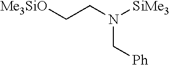 7.27 mmol | 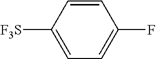 7.27 mmol | CH₂Cl₂ 5 mL | r.t., 1 h | 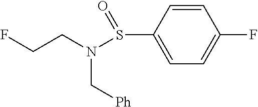 I-40 | 89% |
| 42 | 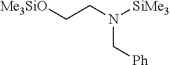 6.19 mmol | 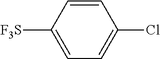 6.19 mmol | CH₂Cl₂ 5 mL | r.t., 1 h | 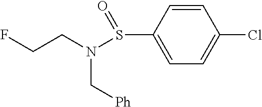 I-41 | 92% |
| 43 | 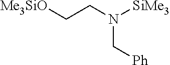 3.82 mmol | 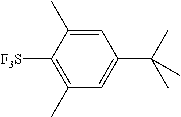 3.82 mmol | heptane 25 mL | 100° C., 0.5 h Addition method | 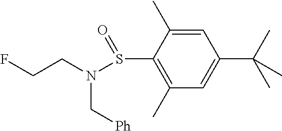 I-42 | <65% |

The properties and spectral data of the products are shown in the following:

N-(2-Fluoroethyl)-N-methyl-benzenesulfinamide (I-36): ¹H-NMR (CDCl₃) δ 2.54 (s, 3H), 3.2-3.4 (m, 2H), 4.40 (dm, 2H, J=44 Hz) 7.2-7.6 (m, 5H); ¹⁹F-NMR (CDCl₃) δ −220.91 (tt, 1F, J=45, 26 Hz); ¹³C-NMR: (CDCl₃): δ 33.84, 52.82 (d, J=21 Hz), 81.84 (d, J=170.5 Hz), 126.11, 128.93, 131.02, 143.57.

N-(2-Fluoroethyl)-N-methyl-p-fluorobenzenesulfinamide (I-37): ¹H-NMR (CDCl₃) δ 2.54 (s, 3H), 3.2-3.4 (m, 2H), 4.45 (dm, 2H, J=47.1 Hz) 7.08 (t, 2H, J=8.6 Hz), 7.54 (m, 2H); ¹⁹F-NMR (CDCl₃) δ −109.22 (m, 1F), −220.11 (tt, 1F, J=48.4, 24.2 Hz); ¹³C-NMR (CDCl₃) δ 33.50, 52.14 (d, J=20.2 Hz), 82.05 (d, J=170.5 Hz), 116.1 (d, J=22.4 Hz), 128.50 (d, J=8.7 Hz), 139.28 (d, J=2.8 Hz), 164.36 (d, J=251.4 Hz).

N-(2-Fluoroethyl)-N-methyl-p-chlorobenzenesulfinamide (I-38): ¹H-NMR (CDCl₃) δ 2.49 (s, 3H), 3.2-3.4 (m, 2H), 4.40 (dm, 2H, J=47.1 Hz) 7.20-7.50 (m, 4H); ¹⁹F-NMR (CDCl₃) δ −220.95 (tt, 1F, J=47.5, 26 Hz); ¹³C-NMR (CDCl₃) δ 33.52, 52.18 (d, J=21 Hz), 81.77 (d, J=170.5 Hz), 127.67, 129.13, 137.23, 142.24.

N-Benzyl-(2-fluoroethyl)-benzenesulfinamide (I-39): ¹H-NMR (CDCl₃) δ 3.2-3.4 (m, 2H), 4.1-4.4 (m, 4H), 7.1-7.8 (m, 10H), 1.28 (s, 9H), 2.62 (s, 6H), 4.56 (m, 2H), 4.64 (td, 2H, J=47, 4 Hz), 7.04 (s, 2H); ¹⁹F-NMR (CDCl₃) δ −220.51 (tt, 1F, J=47, 25.4 Hz); ¹³C-NMR (CDCl₃): δ 47.77 (d, J=21 Hz), 52.54, 82.12 (d, J=170.5 Hz), 126.27, 127.88, 128.73, 128.92, 129.07, 131.24, 136.41, 144.09.

N-Benzyl-(2-fluoroethyl)-p-fluorobenzenesulfinamide (I-40): ¹H-NMR (CDCl₃) δ 3.1-3.4 (m, 2H), 4.1-4.5 (m, 4H), 7.1-7.3 (m, 7H), 7.6-7.7 (m, 2H); ¹⁹F-NMR (CDCl₃) δ −108.69 (s, 1F), −220.77 (tt, 1F, J=47, 25.5 Hz); ¹³C-NMR (CDCl₃) δ 47.81 (d, J=20.2 Hz), 52.17, 81.89 (d, J=170.5 Hz), 116.24 (d, J=22.4 Hz), 127.94, 128.58, 128.71, 128.77, 128.86, 136.20, 139.68 (d, J=2.2 Hz), 164.47 (d, J=251.4 Hz).

N-Benzyl-(2-fluoroethyl)-p-chlorobenzenesulfinamide (I-41): ¹H-NMR (CDCl₃) δ 3.11-3.45 (m, 2H), 4.1-4.52 (m, 4H), 7.1-7.4 (m, 5H), 7.44 (d, 2H, J=8.3 Hz), −7.65 (d, 2H, J=8.3 Hz); ¹⁹F-NMR (CDCl₃) δ −220.73 (tt, 1F, J=47, 25 Hz); ¹³C-NMR (CDCl₃) δ 47.90 (d, J=21 Hz), 52.34, 81.89 (d, J=170.5 Hz), 127.84, 127.99, 128.81, 128.89, 129.32, 136.12, 137.53.

N-Benzyl-(2-fluoroethyl)-4-tert-butyl-2,6-dimethylbenzenesulfinamide (I-42): ¹H-NMR (CDCl₃) δ 1.30 (s, 9H), 2.59 (s, 6H), 3.42 (td, 2H, J=24.4, 5.5 Hz), 4.23-4.51 (m, 4H) 7.04 (s, 2H), 7.21-7.31 (m, 5H); ¹⁹F-NMR (CDCl₃) δ −219.68 (m, 1F); ¹³C-NMR (CDCl₃) δ 20.34, 31.18, 34.58, 48.66 (d, J=21.6 Hz), 53.27, 82.24 (d, J=170.5 Hz), 127.74, 127.83, 128.65, 128.88, 138.03, 153.89.

### Example 44

### Preparation of 3-fluoro-1-phenylsulfinyl-pyrrolidine (I-43)

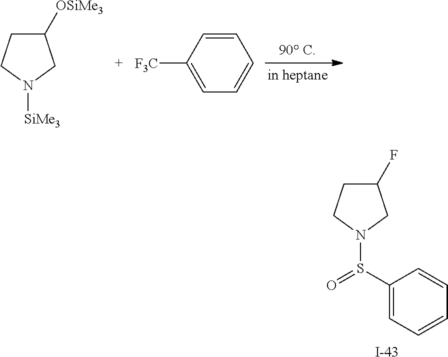

Each of two reactants (4.63 mmol each) was dissolved in equal volume of heptane (2 ml each). Each solution of the reactants was separately and simultaneously added through a Teflon tube with a syringe pump to a stirred 20 mL dry heptane in a fluoropolymer (PFA) vessel heated on 90° C. oil bath. The addition took 1 hour. After addition, the reaction mixture was further stirred for 0.5 hour at 90° C. After cooling, insoluble black resin was removed, and the solution was washed with aqueous sodium bicarbonate solution, dried on magnesium sulfate, and filtered. Removal of solvent at reduced pressure gave 3-fluoro-1-phenylsulfinylpyrroline (I-43) as a 1:0.88 mixture of stereoisomers: Yield, about 60%; ¹H-NMR (CDCl₃) δ 2.10-2.29 (m, 2H), 3.0-3.65 (m, 4H), 5.20 (dm, 1H), 7.25-7.80 (m, 5H); ¹⁹F-NMR (CDCl₃) δ −174.74 (m, 1F); ¹³C-NMR (CDCl₃) δ 43.40, 50.34 (d, J=20 Hz), 52.27 (d, J=20 Hz), 93.15 (d, J=176.8 Hz), 125.49, 128.97, 130.71, 136.58,

### Example 45

### Preparation of 3-fluoro-1-(4-tert-butyl-2,6-dimethylphenylsulfinyl)-pyrrolidine (I-44)

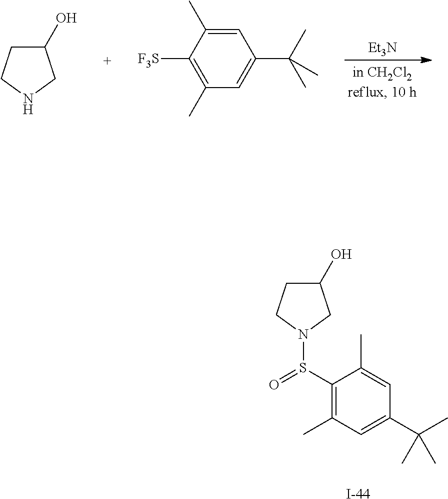

4-tert-Butyl-2,6-dimethylphenylsulfur trifluoride (6.0 mmol) was taken in a Teflon vessel and dissolved in 20 ml of dry dichloromethane. Triethylamine (12.1 mmol) was added at room temperature followed by the addition of a solution of (±)-3-pyrrolidinol (6.0 mmol) in 5 ml of dry dichloromethane. The reaction mixture was refluxed for 10 hours. After cooling to room temperature, the reaction mixture was poured in aqueous sodium carbonate solution. The mixture was extracted with dichloromethane, and the organic layer was separated and dried on magnesium sulfate. After filtration, the filtrate was evaporated to dryness and the residue was column-chromatographed on silica gel using a 2:1 mixture of ether and hexane to give 1-(4-tert-butyl-2,6-dimethylphenylsulfinyl)-3-fluoropyrrolidine (I-44): Yield 72%. ¹⁹F NMR showed that the product was a 1:0.66 mixture of two stereoisomers. ¹⁹F-NMR (CDCl₃) δ −173.28 (m) and −174.44 (m) in an integral ratio of 1:0.66; ¹H-NMR (CDCl₃) δ 1.27 (s, 9H), 2.10-2.20 (m, 2H), 2.53 (s, 6H), 3.10-3.70 (m, 4H), 4.90-5.50 (m, 1H), 6.98 (s, 2H); ¹³C-NMR (CDCl₃) major isomer: δ 19.98, 31.17, 34.48, 43.95, 53.43, (d, J=23.8 Hz), 92.97 (d, J=178.4 Hz) 127.48, 134.63, 137.44, 153.32, minor isomer: δ 19.98, 31.13, 34.48, 46.24, 51.60 (d, J=23.8 Hz), 93.22 (d, J=178.4 Hz) 127.48, 134.94, 137.35, 153.32. Elemental analysis calculated for C₁₆H₂₄FNOS: C, 64.61%; H, 8.13%; N, 4.71%. Found; C, 64.08%; H, 8.21%; N, 4.53%.

### Example 46

### Preparation of 2-fluoromethyl-1-(phenylsulfinyl)pyrrolidine (I-45)

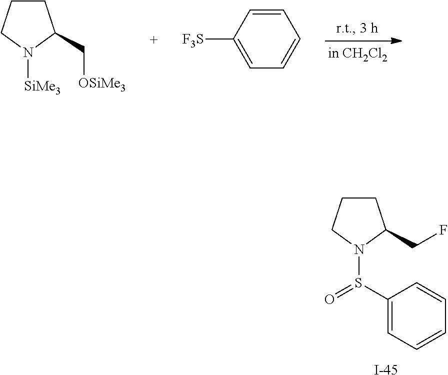

A solution of 3.55 mmol of 1-trimethylsilyl-2-[trimethylsilyloxy)methyl]-pyrrolidine in 2.5 mL of dry dichloromethane was slowly added to a solution of 3.55 mmol of phenylsulfur trifluoride in 2.5 mL of dry dichloromethane at room temperature. 1-Trimethylsilyl-2-[trimethylsilyloxy)methyl]-pyrrolidine was prepared from (s)-(+)-2-pyrrolidinemethanol by a usual method. The reaction mixture was stirred at room temperature for 3 hours, and poured on aqueous sodium carbonate solution and extracted with dichloromethane. The organic layer was dried over anhydrous magnesium sulfate and filitered. Romovel of solvent at reduced pressure gave 2-fluoromethyl-(1-phenylsulfinyl)-pyrrolidine (I-45): Yield 78%. The product was a 6.25:1 mixture of two stereoisomers. Spectral data for a major isomer are shown in the following: ¹H-NMR (CDCl₃) δ 1.60-1.90 (m, 4H), 3.2-4.04 (m, 3H), 3.2-3.4 (m, 2H), 4.20-4.50 (dm, 2H, J=51.5 Hz) 7.00-7.70 (m, 5H); ¹⁹F-NMR (CDCl₃) δ −224 (dt, 1F, J=47.5, 17.3 Hz); ¹³C-NMR (CDCl₃) δ 24.94, 28.40, 42.12, 60.51 (d, J=20 Hz), 84.83 (d, J=174 Hz), 125.83, 128.94, 130.72, 144.38.

### Example 47

### Preparation of Phenylsulfur Chlorotetrafluoride (IV-1)

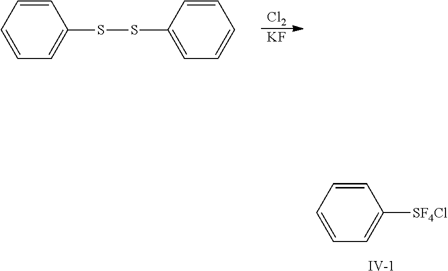

A 500 mL round bottom glassware flask was charged with diphenyl disulfide (33.0 g, 0.15 mol), dry potassium fluoride (KF) (140 g, 2.4 mol) and 300 mL of dry acetonitrile. The stirred reaction mixture was cooled on an ice/water bath under a flow of nitrogen (N₂) (18 mL/min). After N₂ was stopped, chlorine (Cl₂) was bubbled into a reaction mixture at the rate of about 70 mL/min. The Cl₂ bubbling took about 6.5 hours. The total amount of Cl₂ used was about 1.2 mol. After Cl₂ was stopped, the reaction mixture was stirred for additional 3 hours. N₂ was then bubbled through for 2 hours to remove an excess of Cl₂. The reaction mixture was then filtered with 100 mL of dry hexane in air. About 1 g of dry KF was added to the filtrate. The KF restrains possible decomposition of the product. The filtrate was evaporated under vacuum and the resulting residue was distilled at reduced pressure to give a colorless liquid (58.0 g, 88%) of phenylsulfur chlorotetrafluoride (IV-1): b.p. 80° C./20 mmHg; ¹H NMR (CD₃CN) 7.79-7.75 (m, 2H, aromatic), 7.53-7.49 (m, 3H, aromatic); ¹⁹F NMR (CD₃CN) 136.7 (s, SF₄Cl). The NMR analysis showed phenylsulfur chlorotetrafluoride obtained is a trans isomer.

### Examples 48˜62

### Preparation of Arylsulfur Chlorotetrafluorides (IV-1˜12)

Substituted and unsubstituted arylsulfur chlorotetrafluorides (IV-1˜12) were synthesized from sulfur compounds shown in Table 5 by a similar procedure as in Example 47. Table 5 shows the synthesis of the substituted and unsubstituted arylsulfur chlorotetrafluorides (IV-1˜12). Table 5 also shows the starting materials and other chemicals, solvents, reaction conditions, and results, together with those of Example 47.

TABLE 5
Preparation of arylsulfur chlorotetrafluorides (IV-1)~(IV-12)

| Ex. | Starting material | Halogen | Metal fluoride | Solvent | Conditions | (IV) | Yield |
|---|---|---|---|---|---|---|---|
| 47 | 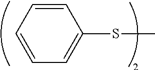 33.0 g (0.15 mol) | Cl₂ ~1.2 mol | KF 140 g (2.4 mol) | CH₃CN 300 mL | 0~5° C. ~9.5 h | 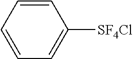 58 g IV-1 | 88% |
| 48 | 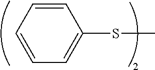 21.8 g (0.1 mol) | Cl₂ 0.68 mol | CsF 243 g (1.6 mol) | CH₃CN 200 mL | 0~5° C. 4 h and r.t. overnight | 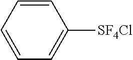 36.3 g IV-1 | 83% |
| 49 | 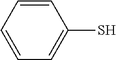 10 g (0.1 mol) | Cl₂ 0.68 mol | KF 47.5 g (0.81 mol) | CH₃CN 100 mL | 6~10° C. 3.7 h | 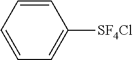 16.6 g IV-1 | 83% |
| 50 | 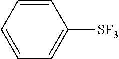 5.0 g (30.1 mmol) | Cl₂ 65.5 mol | CsF 8.74 g (150 mmol) | CH₃CN 20 mL | 6~9° C. 0.7 h | 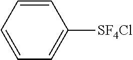 5.62 g IV-1 | 84% |
| 51 | 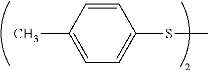 123 g (0.5 mol) | Cl₂ 0.73 mol | KF 464 g (8 mol) | CH₃CN 1L | 0° C. 10.5 h | 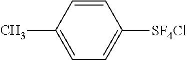 170 g IV-2 | 73% |
| 52 | 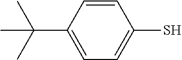 10 g (0.060 mol) | Cl₂ 0.45 mol | CsF 91.6 g (0.602 mol) | CH₃CN 150 mL | 5~10° C. 3.5 h and r.t. 24 h | 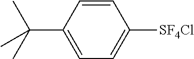 14 g IV-3 | 84% |
| 53 | 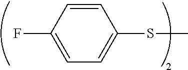 10.0 g (0.039 mol) | Cl₂ 0.28 mol | KF 36 g (0.63 mol) | CH₃CN 100 mL | 0~5° C. 2.5 h and r.t. overnight | 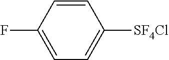 12.5 g IV-4 | 67% |
| 54 | 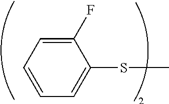 10.0 g (0.039 mol) | Cl₂ 0.31 mol | KF 36.5 g (0.63 mol) | CH₃CN 100 mL | 0~5° C. 1.8 h and r.t. overnight | 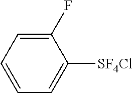 14.9 g IV-5 | 80% |
| 55 | 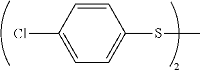 25 g (0.087 mol) | Cl₂ 0.57 mol | KF 86 g (1.48 mol) | CH₃CN 200 mL | 5~8° C. 3.5 h | 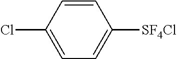 39.5 g IV-6 | 88% |
| 56 | 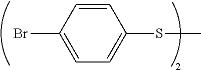 37.6 g (0.1 mol) | Cl₂ 0.72 mol | KF 94 g (1.6 mol) | CH₃CN 200 mL | 0~5° C. 4.5 h and r.t. overnight | 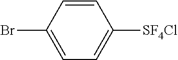 46.2 g IV-7 | 77% |
| 57 | 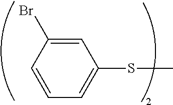 47.7 g (0.127 mol) | Cl₂ 0.88 mol | KF 118 g (2.0 mol) | CH₃CN 250 mL | 0~5° C. 5.5 h and r.t. overnight | 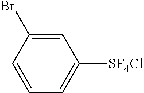 65.7 g IV-8 | 86% |
| 58 | 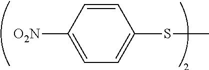 30.8 g (0.1 mol) | Cl₂ 0.72 mol | KF 94 g (1.6 mol) | CH₃CN 200 mL | 0~5° C. 4.5 h and r.t. overnight | 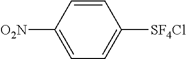 32 g IV-9 | 60% |
| 59 | 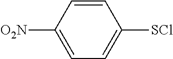 5.0 g (26.4 mmol) | Cl₂ 0.72 mol | KF 15.3 g (264 mol) | CH₃CN 40 mL | 5~11° C. 1.1 h | 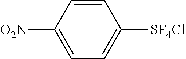 4.69 g IV-9 | 76% |
| 60 | 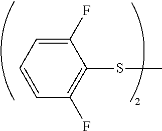 29.1 g (0.1 mol) | Cl₂ ~1.02 mol | CsF 279 g (1.83 mol) | CH₃CN 200 mL | 0~5° C. 5 h and r.t. overnight | 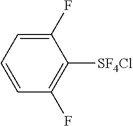 42.3 g IV-10 | 82% |
| 61 | 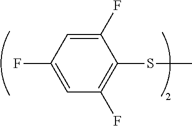 22.9 g (0.07 mol) | Cl₂ ~1.08 mol | KF 90 g (1.55 mol) | CH₃CN 300 mL | 0~5° C. 6 h and r.t. overnight | 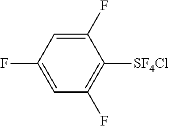 25.8 g IV-11 | 67% |
| 62 | 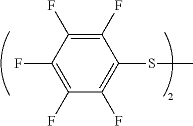 26.1 g (0.065 mol) | Cl₂ ~1 mol | KF 82 g (1.41 mol) | CH₃CN 300 mL | 0~5° C. 5 h and r.t. overnight | 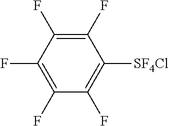 34.9 g IV-12 | 86% |

The properties and spectral data of the product (IV-1) obtained by Examples 47-50 are shown in Example 47. The properties and spectral data of the products obtained by Examples 51-62 are shown by the following

p-Methylphenylsulfur chlorotetrafluoride (IV-2): b.p. 74-75° C./5 mmHg; ¹H NMR (CD₃CN) δ 7.65 (d, 2H, aromatic), 7.29 (d, 2H, aromatic), 2.36 (s, 3H, CH₃); ¹⁹F NMR (CD₃CN) δ 137.66 (s, SF₄Cl); High resolution mass spectrum; found 235.986234 (34.9%) (calcd for C₇H₇F₄S³⁷Cl; 235.986363), found 233.989763 (75.6%) (calcd for C₇H₇F₄S³⁵Cl; 233.989313). The NMR shows that p-methylphenylsulfur chlorotetrafluoride obtained is a trans isomer.

p-(tert-Butyl)phenylsulfur chlorotetrafluoride (IV-3): b.p. 98° C./0.3 mmHg; m.p. 93° C.; ¹H NMR (CDCl₃) δ 1.32 (s, 9H, C(CH₃)₃), 7.43 (d, J=9.2 Hz, 2H, aromatic), 7.64 (d, J=9.2 Hz, 2H, aromatic); ¹⁹F NMR δ 138.3 (s, SF₄Cl). High resolution mass spectrum; found 278.034576 (8.8%) (calcd for C₁₀H₁₃³⁷ClF₄S; 278.033313), found 276.037526 (24.7%) (calcd for C₁₀H₁₃³⁵ClF₄S; 276.036263). Elemental analysis; Calcd for C₁₀H₁₃ClF₄S; C, 43.40%; H, 4.74%. Found; C, 43.69%, H, 4.74%. The NMR showed that p-(t-butyl)phenylsulfur chlorotetrafluoride was obtained as a trans isomer.

p-Fluorophenylsulfur chlorotetrafluoride (IV-4): b.p. 60° C./8 mmHg; ¹H NMR (CD₃CN) 7.85-7.78 (m, 2H, aromatic), 7.25-7.15 (m, 2H, aromatic); ¹⁹F NMR (CD₃CN) 137.6 (s, SF₄Cl), −108.3 (s, CF); High resolution mass spectrum; found 239.961355 (37.4%) (calcd for C₆H₄F₅S³⁷Cl; 239.961291), found 237.964201(100%) (calcd for C₆H₄F₅S³⁵Cl; 237.964241). The NMR shows that p-fluorophenylsulfur chlorotetrafluoride obtained is a trans isomer.

o-Fluorophenylsulfur chlorotetrafluoride (IV-5): b.p. 96-97° C./20 mmHg; ¹H NMR (CD₃CN) 7.77-7.72 (m, 1H, aromatic), 7.60-7.40 (m, 1H, aromatic), 7.25-7.10 (m, 2H, aromatic); ¹⁹F NMR (CD₃CN) 140.9 (d, SF₄Cl), −107.6 (s, CF); High resolution mass spectrum; found 239.961474 (25.4%) (calcd for C₆H₄F₅S³⁷Cl; 239.961291), found 237.964375 (69.8%) (calcd for C₆H₄F₅S³⁵Cl; 237.964241). The NMR shows that o-fluorophenylsulfur chlorotetrafluoride obtained is a trans isomer.

p-Chlorophenylsulfur chlorotetrafluoride (IV-6): b.p. 65-66° C./2 mmHg; ¹H NMR (CDCl₃) δ 7.38 (d, 2H, J=9.1 Hz), 7.65 (d, 2H, J=9.1 Hz); ¹⁹F NMR (CDCl₃) 137.4 (s, 4F, SF₄Cl). High resolution mass spectrum; found 257.927507 (13.3%) (calcd for C₆H₄F₄S³⁷Cl₂; 257.928790), found 255.930746 (68.9%) (calcd for C₆H₄F₄S³⁷Cl³⁵Cl; 255.931740), found 253.933767 (100.0%) (calcd for C₆H₄F₄S³⁵Cl₂; 253.934690). The NMR showed that p-chlorophenylsulfur chlorotetrafluoride obtained is a trans isomer.

p-Bromophenylsulfur chlorotetrafluoride (IV-7): m.p. 58-59° C.; ¹H NMR (CD₃CN) δ 7.67 (s, 4H, aromatic); ¹⁹F NMR (CD₃CN) δ 136.56 (s, SF₄Cl); High resolution mass spectrum; found 301.877066 (16.5%) (calcd for C₆H₄⁸¹Br³⁷ClF₄S; 301.879178), found 299.880655 (76.6%) (calcd for C₆H₄⁸¹Br³⁵ClF₄S; 299.881224 and calcd for C₆H₄⁷⁹Br³⁷ClF₄S; 299.882128), found 297.882761 (77.4%) (calcd for C₆H₄⁷⁹Br³⁵ClF₄S; 297.884174).

Elemental analysis; calcd for C₆H₄BrClF₄S; C, 24.06%; H, 1.35%; found, C, 24.37%; H, 1.54%. The NMR showed that p-bromophenylsulfur chlorotetrafluoride was obtained as a trans isomer.

m-Bromophenylsulfur chlorotetrafluoride (IV-8): b.p. 57-59° C./0.8 mmHg; ¹H NMR (CD₃CN) 7.90-7.88 (m, 1H, aromatic), 7.70-7.50 (m, 2H, aromatic), 7.40-7.30 (m, 1H, aromatic); ¹⁹F NMR (CD₃CN) 136.74 (s, SF₄Cl). High resolution mass spectrum; found 301.878031 (29.1%) (calcd for C₆H₄⁸¹Br³⁷ClF₄S; 301.879178), found 299.881066 (100%) (calcd for C₆H₄⁸¹Br³⁵ClF₄S; 299.881224 and calcd for C₆H₄⁷⁹Br³⁷ClF₄S; 299.882128), found 297.883275 (77.4%) (calcd for C₆H₄⁷⁹Br³⁵ClF₄S; 297.884174). The NMR showed that m-bromophenylsulfur chlorotetrafluoride obtained was a trans isomer.

p-Nitrophenylsulfur chlorotetrafluoride (IV-9): m.p. 130-131° C.; ¹H NMR (CD₃CN) 8.29 (d, J=7.8 Hz, 2H, aromatic), 8.02 (d, J=7.8 Hz, 2H, aromatic); ¹⁹F NMR (CD₃CN) 134.96 (s, SF₄Cl); High resolution mass spectrum; found 266.956490 (38.4%) (calcd for C₆H₄³⁷ClF₄NO₂S; 266.955791), found 264.959223 (100%) (calcd for C₆H₄³⁵ClF₄NO₂S; 264.958741). Elemental analysis; calcd for C₆H₄ClF₄NO₂S; C, 27.13%; H, 1.52%; N, 5.27%; found, C, 27.16%; H, 1.74%; N, 4.91%. The NMR shows that p-nitrophenylsulfur chlorotetrafluoride obtained is a trans isomer.

2,6-Difluorophenylsulfur chlorotetrafluoride (IV-10): The product (b.p. 120-122° C./95-100 mmHg) obtained from Example 14 is a 6:1 mixture of trans- and cis-isomers of 2,6-difluorophenylsulfur chlorotetrafluoride. The trans-isomer was isolated as pure form by crystallization; mp. 47.6-48.3° C.; ¹⁹F NMR (CDCl₃) δ 143.9 (t, J=26.0 Hz, 4F, SF₄), −104.1 (quintet, J=26.0 Hz, 2F, 2,6-F): ¹H NMR (CDCl₃) δ 6.97-7.09 (m, 2H, 3,5-H), 7.43-7.55 (m, 1H, 4-H); ¹³C NMR (CDCl₃) δ 157.20 (d, J=262.3 Hz), 133.74 (t, J=11.6 Hz), 130.60 (m), 113.46 (d, J=14.6 Hz); high resolution mass spectrum; found 257.950876 (37.6%) (calcd for C₆H₃³⁷ClF₆S; 257.951869), found 255.955740 (100%) (calcd for C₆H₃³⁵ClF₆S; 255.954819); elemental analysis; calcd for C₆H₃ClF₆S; C, 28.08%, H, 1.18%; found; C, 28.24%, H, 1.24%. The cis-isomer was assigned in the following; ¹⁹F NMR (CDCl₃) δ 158.2 (quartet, J=161.8 Hz, 1F, SF), 121.9 (m, 2F, SF₂), 76.0 (m, 1F, SF). The ¹⁹F NMR assignment of aromatic fluorine atoms of the cis-isomer could not be done because of possible overlapping of the peaks of the trans-isomer.

2,4,6-Trifluorophenylsulfur chlorotetrafluoride (IV-11): trans-isomer; m.p. 55.8-56.7° C.; ¹⁹F NMR (CDCl₃) δ 144.07 (t, J=26.0 Hz, 4F, SF₄), −99.80 (t, J=26.0 Hz, 2F, o-F), −100.35 (s, 1F, p-F); ¹H NMR (CDCl₃) δ 6.79 (t, J=17.5 Hz, m-H); ¹³C NMR (CDCl₃) δ 164.16 (dt, J=164.2 Hz, 15.2 Hz, 4-C), 158.18 (dm, J=260.7 Hz, 2-C), 127.7 (m, 1-C), 102.1 (tm, J=27.8 Hz, 3-C). Elemental analysis; calcd for C₆H₂ClF₇S; C, 26.24%; H, 0.73%; found, C, 26.23%; H, 1.01%. The NMR shows that 2,4,6-trifluorophenylsulfur chlorotetrafluoride obtained is a trans isomer.

2,3,4,5,6-Pentafluorophenylsulfur chlorotetrafluoride (IV-12): The product (b.p. 95-112° C./100 mmHg) obtained from Experiment 16 was a 1.7:1 mixture of trans and cis isomers of 2,3,4,5,6-pentafluorophenylsulfur chlorotetrafluoride. The isomers were assigned by ¹⁹F NMR: The trans isomer; ¹⁹F NMR (CDCl₃) δ 144.10 (t, J=26.0 Hz, 4F, SF₄), −132.7 (m, 2F, 2,6-F), −146.6 (m, 1F, 4-F), −158.9 (m, 2F, 3,5-F); ¹³C NMR (CDCl₃) δ 143.5 (dm, J=265.2 Hz), 141.7 (dm, J=263.7 Hz), 128.3 (m). The cis isomer; ¹⁹F NMR (CDCl₃) δ 152.39 (quartet, J=158.9 Hz, 1F, SF), 124.32 (m, 2F, SF₂), 79.4 (m, 1F, SF), −132.7 (m, 2F, 2,6-F), −146.6 (m, 1F, 4-F), −158.9 (m, 2F, 3,5-F). High resolution mass spectrum of a 1.7:1 mixture of the trans and cis isomers; found 311.923124 (15.5%) (calcd for C₆³⁷ClF₉S; 311.923604), found 309.926404 (43.1%) (calcd for C₆³⁵ClF₉S; 309.926554).

### Example 63

### Preparation of Fluoroalkyl Benzenesulfinate (I-1) by Two-Step Method from Phenylsulfur Chlorotetrafluoride by Action of Pyridine as a Reducing Substance

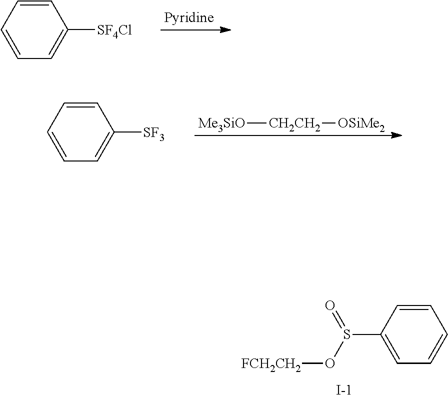

Into a solution of 2.04 mmol of phenylsulfur chlorotrifluoride in 5 mL of dry dichloromethane in a fluoropolymer vessel, was added 4.08 mmol of pyridine. The reaction mixture was stirred at room temperature for 1.5 hours. ¹⁹F NMR showed complete consumption of phenylsulfur chlorotetrafluoride and formation of phenylsulfur trifluoride. Into the reaction mixture, a solution of 2.00 mmol of bis(trimethylsilyl ether) of ethylene glycol in 1 mL of dry dichloromethane was slowly added during about 20 minutes. The reaction mixture was stirred for 2 hours and poured on aqueous sodium carbonate solution. The organic layer was washed with water and dried over anhydrous magnesium sulfate and filtered. Removal of solvent at reduced pressure gave 2-fluoroethyl phenylsulfinate (I-1).

### Examples 64-67

### Preparation of Fluoroalkyl Arenesulfinate by a Two-Step Method from arylsulfur Halotetrafluoride by Action of a Reducing Substance

Various fluoroalkyl arenesufinates were prepared in a similar manner as described in Example 63. The results and reaction conditions are shown in Table 6 together wth the results and reaction conditions of Example 63.

TABLE 6
Preparation of fluoroalkyl arenesulfinates by two-step methods from arylsulfur halotetrafluorides and a reducing substance

| | Step 1 | | | | Step 2 | | | |
|---|---|---|---|---|---|---|---|---|
| | | | Solvent | | | Soovent | | |
| | | | Temp- | | | Temp- | | |
| | | Reduding | erature | | | erature | | |
| Ex. | (IV) | substancce | Time | (III) | (II) | Time | (I) | Yield |
| 63 | 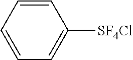 2.04 mmol | Pyridine 4.08 mmol | r.t 1.5 h CH₂Cl₂ 5 mL | 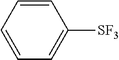 | 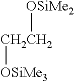 4.13 mmol | r.t. 2 h CH₂Cl₂ 6 mL | 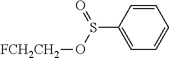 I-1 | 80% |
| 64 | 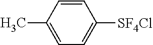 5.27 mmol | Pyridine 10.5 mmol | r.t 1.5 h CH₂Cl₂ 5 mL | 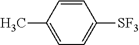 | 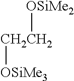 5.20 mmol | r.t. 2 h CH₂Cl₂ 6 mL | 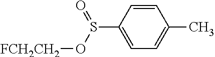 I-3 | 72% |
| 65 | 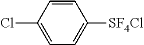 4.93 mmol | Pyridine 9.87 mmol | r.t 1.5 h CH₂Cl₂ 5 mL | 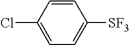 | 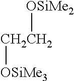 5.20 mmol | r.t. 2 h CH₂Cl₂ 6 mL | 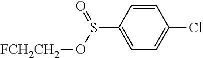 I-5 | 76% |
| 66 | 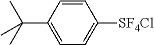 4.44 mmol | Pyridine 8.88 mmol | r.t 1.5 h CH₂Cl₂ 5 mL | 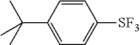 | 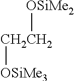 4.3 mmol | r.t. 2 h CH₂Cl₂ 6 mL | 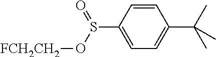 I-46 | 70% |
| 67 | 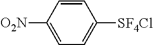 4.24 mmol | Pyridine 8.28 mmol | r.t 1.5 h CH₂Cl₂ 5 mL | 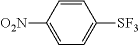 | 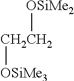 4.20 mmol | r.t. 2 h CH₂Cl₂ 6 mL | 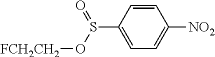 I-47 | 75% |

Spectral data of I-1, I-3 and I-5 are as shown before. Data of I-46 and I-47 are shown in the following:

2-Fluoroethyl p-(tert-butyl)benzenesulfinate (I-46): ¹H-NMR (CDCl₃) δ 3.75 (m, 1H), 4.20 (m, 1H), 4.48 (dm, 2H, J=47 Hz), 7.51 (m, 3H), 7.63 (m, 2H); ¹⁹F-NMR (CDCl₃) δ −224.15 (tt, J=47.6 Hz, 28.4 Hz, 1F, CH₂F); ¹³C-NMR (CDCl₃) δ: 31.10, 35.30, 63.0 (d, J=20.0 Hz), 81.95 (d, J=172 Hz), 125.19, 126.28, 128.25, 141.18.

2-Fluoroethyl p-nitrophenylsulfinate (I-47): ¹H-NMR (CDCl₃) δ 3.64-3.75 (m, 1H), 3.82-4.0 (m, 1H), 4.20-4.40 (m, 1H), 4.44-4.70 (m, 1H), 7.0-8.0 (m, 2H), 8.35-8.47 (m, 2H); ¹⁹F-NMR (CDCl₃) δ −224.75 (tt, 1F, J=45.5, 28.2 Hz); ¹³C-NMR (CDCl₃) δ 64.59 (d, J=20.2 Hz), 80.58 (q, J=172.5 Hz), 124.48, 124.64, 126.90, 129.47, 150.37, 180.58.

### Example 68

### Preparation of 2-fluoroethyl p-nitrobenzenesulfinate (I-47) by a reaction of Me₃SiOCH₂CH₂OSiMe₃ and p-nitrophenylsulfur chlorotetrafluoride in the presence of a reducing substance (pyridine)

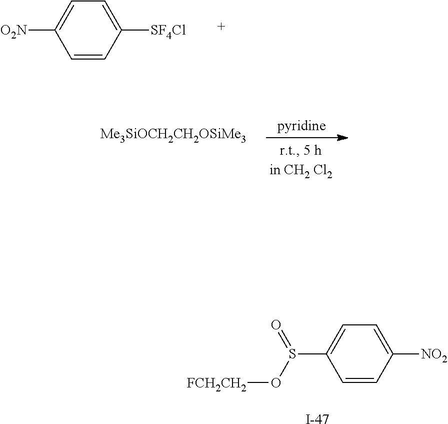

p-Nitrophenylsulfur chlorotetrafluoride (2.0 mmol) and Me₃SiOCH₂CH₂OSiMe₃ (2.0 mmol) were dissolved in 4 ml of dichloromethane in a fluoropolymer (PFA) vessel. Into the solution, pyridine (4.0 mmol) was added at room temperature. The reaction mixture was stirred at room temperature for 5 hours and washed with water. The organic dichloromethane layer is dried over anhydrous magnesium sulfate and filtered. Removal of solvent gave a crude product, which was chromatographed using a 1:8 mixture of ether and hexane as an eluent to give 2-fluoroethyl p-nitrophenylsulfinate (I-47): Yield 60%. The spectral data are shown in Example 67.

### Example 69

### Preparation of 4-tert-butyl-2,6-dimethylphenylsulfinyl fluoride

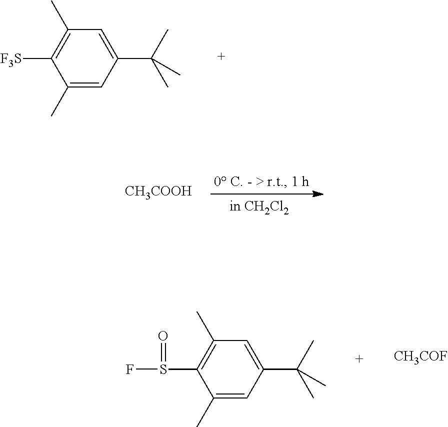

Acetic acid (240 mg, 4 mmol) was added to a stirred solution of 4-tert-butyl-2,6-dimethylphenylsulfur trifluoride (1.00 g, 4.00 mmol) in 5 mL of dichloromethane in a fluoropolymer vessel cooled on an ice bath. The bath was removed after 0.5 hour and the reaction mixture was stirred at room temperature for additional 0.5 hour. ¹⁹F-NMR analysis of the reaction mixture showed that 4-tert-butyl-2,6-dimethylphenylsulfinyl fluoride was produced in 89% yield. After the reaction, the reaction mixture was evaporated to dryness by vacuum pump. 4-tert-Butyl-2,6-dimethylphenylsulfinyl fluoride was obtained as a solid and used for the next reactions (Examples 70 and 71) without further purification. Another product, CH₃COF, was easily removed by vacuum pump because of its low boiling point (bp 20-21° C.).

### Example 70

### Preparation of (2S,4S)—N-(4-tert-butyl-2,6-dimethylphenylsulfinyl)-2-cyano-4-fluoropyrrolidine (I-48)

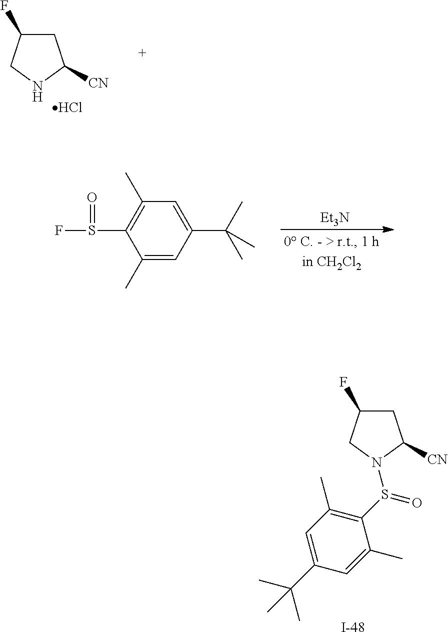

A solution of (2S,4S)-2-cyano-4-fluoropyrrolidine hydrochloride (527 mg, 3.5 mmol) and triethylamine (3.5 mmol) in 2 mL of dichloromethane was slowly added to a stirred solution of 4-tert-Butyl-2,6-dimethylphenylsulfinyl fluoride (799 mg, 3.5 mmol), prepared in Example 53a, and triethylamine (3.5 mmol) in 3 mL of dichloromethane in a fluoropolymer vessel cooled on an ice bath. The bath was removed after 0.5 hour and the reaction mixture was stirred at room temperature for additional 0.5 hour. Into the reaction mixture, were added 15 mL of dichloromethane and 40 mL of aqueous saturated K₂CO₃ solution, and the reaction mixture was stirred at room temperature for 0.5 hour. The organic layer was separated and washed with water, dried with anhydrous magnesium sulfate, and filtered. Removal of the solvent from the filtrate gave a solid, which was then mixed with pentane and stirred for 15 minutes. Pentane was decanted off to obtain an insoluble solid which was the product. The product was further purified by preparative thin-layered chromatography using a 1:1 mixture of pentane and dichloromethane to give 902 mg (80%) of (2S,4S)—N-(4-tert-butyl-2,6-dimethylphenylsulfinyl)-2-cyano-4-fluoropyrrolidine (I-48): ¹⁹F NMR (CDCl₃) (a mixture of stereoisomers) δ −174 to 176 (m, 1F); ¹H-NMR (CDCl₃) δ 1.28 (s, 9H), 2.20-2.8 (m, 8H), 3.0-4.0 (m, 2H), 4.68 (m, 1H), 5.26 (dm, 1H, J=53 Hz), 7.10 (s, 2H); ¹³C-NMR (CDCl₃) (a major stereoisomer) δ 19.93, 31.11, 34.64, 39.30 (d, J=21 Hz), 49.20, 35.06, 55.21 (d, J=23.8 Hz), 92.40 (d, J=180.5 Hz), 117.91, 128.12, 133.01, 137.66, 154.88; (a minor stereoisomer) δ 19.86, 31.11, 34.64, 38.9 (d, J=21 Hz), 49.51, 55.21 (d, J=23.8 Hz), 92.30 (d, J=180.5 Hz), 118.67, 127.94, 133.32, 137.74, 154.46.

### Example 71

### Preparation of (2S,4S)—N-(4-tert-butyl-2,6-dimethylphenylsulfinyl)-4-fluoro-2-(methoxycarbonyl)pyrrolidine (I-49)

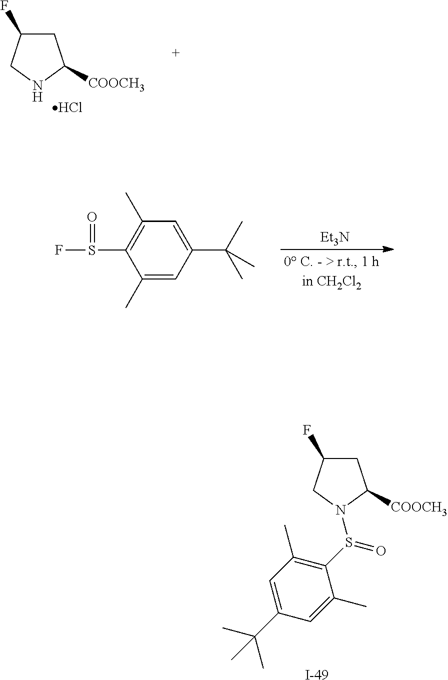

A solution of (2S,4S)-4-fluoro-2-(methoxycarbonyl)pyrrolidine hydrochloride (3.31 g, 18 mmol) and triethylamine (18 mmol) in 10 mL of dichloromethane was slowly added to a stirred solution of 4-tert-Butyl-2,6-dimethylphenylsulfinyl fluoride (5.33 g, 21.3 mmol), prepared in Example 53a, and triethylamine (19 mmol) in 15 mL of dichloromethane in a fluoropolymer vessel cooled on an ice bath. The bath was removed after 0.5 hour and the reaction mixture was stirred at room temperature for additional 0.5 hour. Into the reaction mixture, were added 25 mL of dichloromethane and 50 mL of aqueous saturated K₂CO₃ solution, and the mixture was stirred at room temperature for 0.5 hour. The organic layer was separated and washed with water, dried with anhydrous magnesium sulfate, and filtered. Removal of the solvent from the filtrate gave a solid, which was then washed with pentane to give 5.0 g (78%) of (2S,4S)—N-(4-tert-butyl-2,6-dimethylphenylsulfinyl)-4-fluoro-2-(methoxycarbonyl)pyrrolidine (I-49) as off-white solid: ¹⁹F-NMR (CDCl₃) δ −174.53 (m): ¹H-NMR (CDCl₃) δ 1.27 (s, 9H), 2.20-2.8 (m with s, 8H), 3.0-3.2 (m, 1H), 3.7-4.0 (m with s, 4H), 4.53 (dm, J=10 Hz, 1H), 5.22 (dm, J=52 Hz, 1H), 6.99 (s, 2H): ¹³C-NMR (CDCl₃) δ 19.94, 31.15, 34.53, 38.04 (d, J=21.7 Hz), 50.23 (d, J=23.1 Hz), 52.49, 61.24, 92.93 (d, J=179.9 Hz), 127.75, 134.17, 137.84, 153.84, 172.09.

### Examples 72

### Preparation of 2-fluoroethyl benzenesulfonate by oxidation of 2-fluoroethyl benzenesulfinate

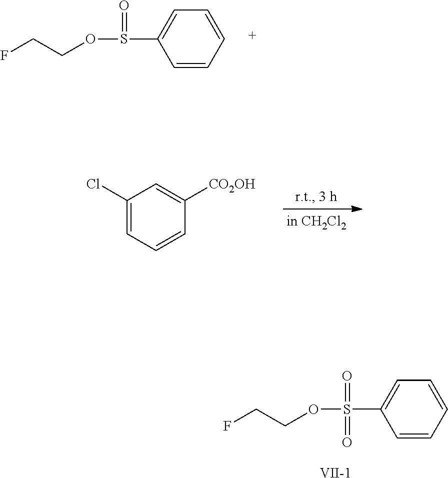

3-Chloroperbenzoic acid (3.7 mmol) was added into a solution of 2-fluoroethyl phenylsulfinae (3.64 mmol) in 10 ml of anhydrous dichloromethane. The mixture was stirred at room temperature for 3 hours. During this period insoluble 3-chlorobenzoic acid is formed. Additional 5 ml of dichloromethane was added to the reaction mixture and the mixture was washed with saturated sodium carbonate aqueous solution and then water and dried over anhydrous MgSO₄. Filtration and removal of solvent at reduced pressure gave 2-fluoroethyl phenylsulfonate (VII-1). Yield 87%; ¹H-NMR (CDCl₃) δ 5.58 (dt, 2H, J=47 Hz, J=2.4 Hz), 4.29 (dt, 2H, J=16 Hz, J=2.4 Hz), 7.5-7.75 (m, 3H), 7.85-8.0 (m, 2H); ¹⁹F-NMR (CDCl₃) δ −224.52 (tt, 1F, J=45, 28 Hz); ¹³C-NMR (CDCl₃) δ 68.77 (d, J=21.0 Hz), 80.60 (d, J=174.0 Hz), 128.01, 129.44, 134.16, 135.72.

### Examples 73-79

### Synthesis of Various Fluoroalkyl Arenesulfonates and Arenesulfonamides from Fluoroalkyl Arenesulfinates and Arenesulfinamides

Various fluoroalkyl arenesulfonates and arenesulfonamides were synthesized by oxidation of the corresponding fluoroalkyl arenesulfinates and arenesulfinamides in the same manner as in Example 72. The results and reaction conditions are shown in Table 7 together with Example 72.

TABLE 7
Preparation of fluoroalkyl arenesulfonates and arenesulfonamides from fluoroalkyl arenesulfinates and arenesulfinamides

| Ex. | (I) | oxidizer | Solvent | Conditions | Sulfonate or sulfonamide | Yield |
|---|---|---|---|---|---|---|
| 72 | 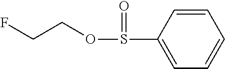 3.64 mmol | 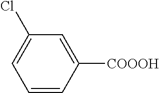 3.7 mmol | CH₂Cl₂ 10 mL | r.t., 3 h | 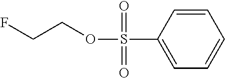 VII-1 | 87% |
| 73 | 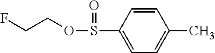 1.50 mmol | 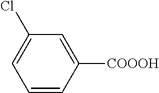 1.60 mmol | CH₂Cl₂ 10 mL | r.t., 3 h | 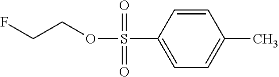 VII-2 | 88% |
| 74 | 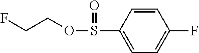 1.72 mmol | 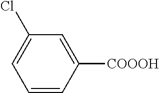 1.82 mmol | CH₂Cl₂ 10 mL | r.t., 3 h | 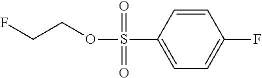 VII-3 | 90% |
| 75 | 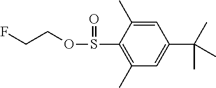 3.33 mmol | 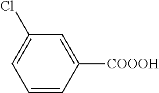 3.43 mmol | CH₂Cl₂ 10 mL | r.t., 3 h | 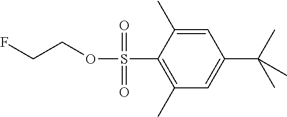 VII-4 | 85% |
| 76 | 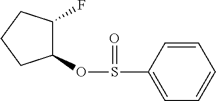 2.5 mmol | 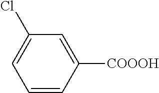 2.6 mmol | CH₂Cl₂ 10 mL | r.t., 3 h | 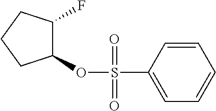 VII-5 | 82% |
| 77 | 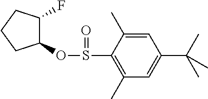 2.9 mmol | 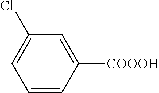 3.1 mmol | CH₂Cl₂ 10 mL | r.t., 3 h | 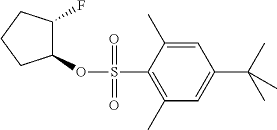 VII-6 | 80% |
| 78 | 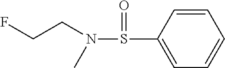 3.57 mmol | 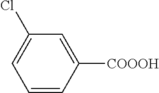 3.67 mmol | CH₂Cl₂ 10 mL | r.t., 3 h | 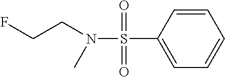 VIII-1 | 90% |
| 79 | 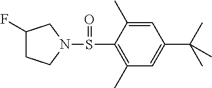 1.0 mmol | 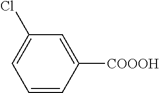 1.1 mmol | CH₂Cl₂ 10 mL | r.t., 3 h | 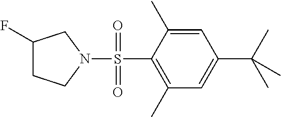 VIII-2 | 85% |

Spectral data of the products are shown in the following:

2-Fluoroethyl p-methylbenzenesulfonate (VII-2): ¹H-NMR (CDCl₃) δ 3.36 (s, 3H), 4.18 (dm, 2H), 4.52 (dt, 2H, J=47.1, 4.1 Hz), 7.30 (d, 2H, J=8.6 Hz), 7.73 (d, 2H, J=8.3 Hz); ¹⁹F-NMR (CDCl₃) δ −224.49 (tt, 1F, J=47.7, 28.1 Hz); ¹³C-NMR (CDCl₃): δ 21.59, 68.94 (d, J=20.2 Hz), 80.74 (d, J=173.4 Hz), 127.94, 130.10, 132.50, 145.41.

2-Fluoroethyl p-fluorobenzenesulfonate (VII-3): ¹H-NMR (CDCl₃) δ 4.24 (dt, 2H, J=27.9, 4.1 Hz), 4.52 (dt, 2H, J=47.1, 4.1 Hz), 7.10-7.30 (m, 2H), 7.80-7.94 (m, 2H); ¹⁹F-NMR (CDCl₃) δ −224.54 (tt, 1F, J=45.5, 28.1 Hz); ¹³C-NMR (CDCl₃) δ 69.24 (d, J=20.2 Hz), 80.68 (d, J=172.7 Hz), 116.67, 116.97, 130.87 (d, J=10.1 Hz), 131.63 (d, J=3.6 Hz).

2-Fluoroethyl 4-tert-butyl-2,6-dimethylbenzenesulfonate (VII-4): ¹H-NMR (CDCl₃) δ 1.30 (s, 9H), 2.66 (s, 6H), 4.22 (dt, 2H, J=29, 4.1 Hz), 4.60 (dt, 2H, J=47, 4.1 Hz), 7.1f (s, 1H); ¹⁹F-NMR (CDCl₃) δ −223.94 (tt, 1F, J=43.4, 26 Hz); ¹³C-NMR (CDCl₃): δ 23.00, 30.95, 34.79, 67.56 (d, J=21 Hz), 80.70 (d, J=173.3 Hz), 128.31, 130.24, 139.87, 156.40.

trans-1-(Phenylsulfonyloxy)-2-fluorocyclopentane (VII-5): ¹H-NMR (CDCl₃) δ 1.50-2.20 (m, 6H), 4.80-5.20 (m, 2H), 7.30-8.20 (m, 5H); ¹⁹F-NMR (CDCl₃) δ −180.13 (m, 1F); ¹³C-NMR (CDCl₃): δ 21.00, 30.25 (d, J=21.0 Hz), 30.27, 85.49 (d, J=31 Hz), 96.69 (d, J=178.8 Hz), 127.91, 129.43, 134.03.

trans-1-(4-tert-butyl-2,6-dimethylphenysulfonyloxy)-2-fluorocyclopentane (VII-6): ¹H-NMR (CDCl₃) δ 1.30 (s, 9H), 1.70-1.85 (, m, 6H), 2.65 (s, 6H), 4.70-8.20 (m, 2H), 7.15 (s, 2H); ¹⁹F-NMR (CDCl₃) δ −179.88 (m, 1F); ¹³C-NMR (CDCl₃) δ 21.10, 23.08, 30.31 (d, J=23.1 Hz), 30.96, 34.78, 84.33 (d, J=30.3 Hz), 97.16 (d, J=177.7 Hz), 128.38, 131.15, 139.60, 156.30.

N-(2-Fluoroethyl)-N-methylbenzenesulfonamide (VIII-1): ¹H-NMR (CDCl₃) δ 2.74 (s, 3H), 3.25 (dt, 2H, J=25.8, 24.8 Hz), 4.45 (dt, 2H, J=47.1, 4.8 Hz), 7.30-7.78 (m, 5H); ¹⁹F-NMR (CDCl₃) δ −220.75 (tt, 1F, J=47.7, 24 Hz); ¹³C-NMR (CDCl₃): δ 36.25, 50.31 (d, J=21.0 Hz), 82.59 (q, J=170.4 Hz), 127.27, 129.30, 132.94, 137.44.

1-(4-tert-Butyl-2,6-dimethylphenylsulfonyl)-3-fluoropyrrolidine (VIII-2): ¹H-NMR (CDCl₃): δ 1.29 (s, 9H), 1.90-2.4 (m, 2H), 3.15-3.65 (m, 4H), 5.25 (dt, 1H, J=53.5, 2.0 Hz); ¹⁹F-NMR (CDCl₃) δ −175.0 (m, 1F); ¹³C-NMR (CDCl₃): δ 23.30. 31.00, 32.85 (d, J=21.7 Hz), 34.66, 44.68, 53.05 (d, J=23.1 Hz), 36.25, 50.31 (d, J=21.0 Hz), 92.64 (d, J=178.4 Hz), 128.44, 132.65, 139.94, 155.40.

### Example 80

### Reaction of 2-fluoroethyl benzenesulfonate with sodium 2-naphtholate

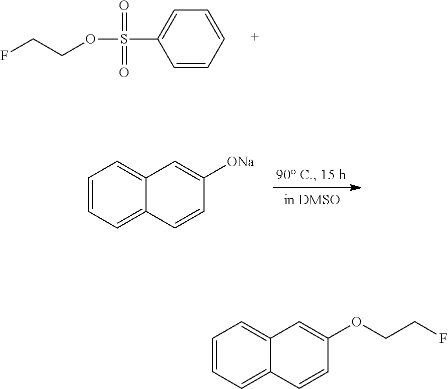

Sodium 2-naptholate was prepared in the following way: Sodium hydride (1.50 mmol, 60% dispersion in mineral oil) was placed in dry flask and washed with 5 ml of anhydrous hexane to remove oil. Anhydrous DMSO (5 ml) was added followed by the addition of 2-naphthol (1.50 mmol) and the mixture was stirred at room temperature for 1 hour. Into the solution of sodium 2-naphtholate, was added a solution of 2-fluoroethyl benzenesulfonate in 1 ml of DMSO. The reaction mixture was heated at 90° C. for 15 hours and cooled to room temperature. After water was added to it, the mixture was extracted with a 1:1 mixture of hexane and ether (25 ml). The extract was washed and dried over anhydrous magnesium sulfate and filtered. Removal of solvent at reduced pressure gave β-(2-fluoroethoxy)naphthaene as white powder solid: Yield 92%; ¹H-NMR (CDCl₃) δ 3.34 (dt, 2H, J=27.9, 4.14 Hz), 4.85 (dt, 2H, J=47.1, 3.0 Hz), 7.10-7.22 (m, 2H), 7.30-7.51 (m, 2H), 7.70-7.84 (m, 3H); ¹⁹F-NMR (CDCl₃) δ −223.50 (tt, 1F, J=47.6, 28.2 Hz); ¹³C-NMR (CDCl₃) δ 67.16 (d, J=20.2 Hz), 82.01 (d, J=170.5 Hz), 106.83, 118.94, 123.98, 126.57, 126.88, 127.79, 129.29, 129.71, 134.48, 156.46.

### Example 81

### Reaction of 2-fluoroethyl 4-tert-butyl-2,6-dimethylbenzenesulfonate with sodium 2-naphtholate

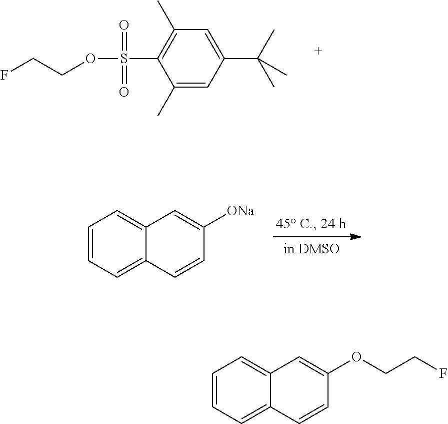

A solution of sodium naphtholate (2 mmol) in DMSO (5 mL) was prepared in the same manner as in Example 80. Into the solution of sodium naphtholate, was added a solution of 2-fluoroethyl 4-tert-butyl-2,6-dimethylbenzenesulfonate (2.0 mmol) in DMSO (5 mL). The reaction mixture was heated at 45° C. for 24 hours and cooled to room temperature. A 1:1 mixture of hexane and ether (25 ml) was added to the reaction mixture and the mixture was washed with water (50 ml). The organic layer was separated out, washed with water (20 ml×2), and dried over anhydrous magnesium sulfate. After filtration, removal of solvent at reduced pressure gave 2-(2-fluoroethoxy)naphthol as white powder solid which was crystallized from hexane: Yield: 75%. The spectral data of the product was shown in Example 80.

### Example 82

### Deprotection of N-benzyl-N-(2-fluoroethyl)benzenesulfinamide

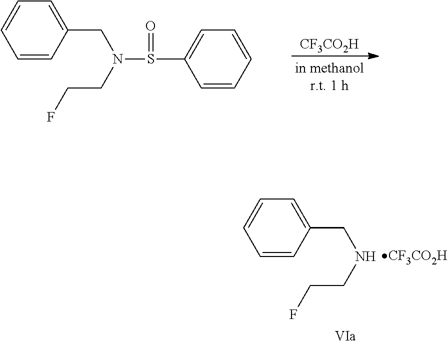

Trifluoroacetic acid (8.0 mmol) was added slowly to a solution of N-benzyl-N-(2-fluoroethyl)benzenesulfinamide in 3 ml of methanol. The reaction mixture was stirred at room temperature for 1 hour. The mixture was concentrated and the residue was column-chromatographed on silica gel (short column). It was eluted with a 3:7 mixture of ethyl acetate and hexane (50 ml) and then methanol (50 ml). The amine salt was eluted with methanol. Removal of methanol at reduced pressure gave a trifluoroacetic acid salt of N-(2-fluoroethyl)benzylamine as a solid powder: Yield 78%; ¹H-NMR (D₂O) δ 3.40 (dt, 2H, J=27.2, 4.5 Hz), 4.61 (dt, 2H, J=50.0, 4.8 Hz), 7.35 (s, 5H); ¹⁹F-NMR (D₂O) δ −75.51 (s, 3F), 224.53 (tt, 1F, J=45.5, 28.1 Hz); ¹³C-NMR (D₂O) δ 46.99 (d, J=56.0 Hz), 51.16, 79.32 (d, J=165.4 Hz), 117.0 (q, J=230 Hz), 129.30, 129.78, 129.85, 120.43, 163.26.

### Example 83

### Deprotection of 1-(4-tert-butyl-2,4-dimethylphenylsulfinyl)-3-fluoroprrolidine; Preparation of 3-fluoropyrrolidine salt

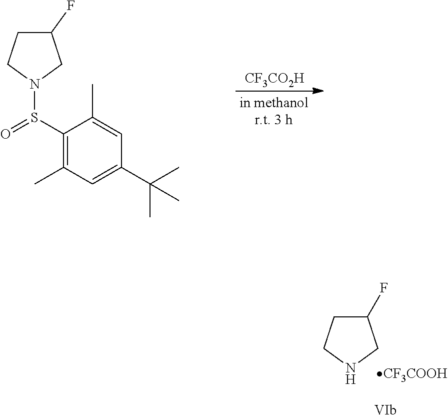

Into a solution of 1-(4-tert-butyl-2,6-dimethylphenylsulfinyl)-3-fluoropyrrolidine (1.0 mmol) in 3 ml of methanol, was added trifluoroacetic acid (8.0 mmol). After it was stirred at room temperature for 3 hours, the reaction mixture was concentrated. The residue was chromatographed on silica gel (short column). It was eluted with a 3:7 mixture of ethyl acetate and hexane (50 ml) and then with methanol (50 ml). The amine salt was eluted with methanol (50 ml). Removal of methanol at reduced pressure gave a trifluoroacetic acid salt of 3-fluoropyrrolidine as a solid powder: Yield 70%; ¹⁹F-NMR (D₂O) δ −75.51 (s, 3F), −176.79 (m, 1F); ¹H-NMR (D₂O) δ 1.90-2.40 (m, 2H), 3.15-3.65 (m, 4H), 5.35 (dt, 1H, J=51.6, 3.8 Hz); ¹³C-NMR (D₂O) δ 31.08 (d, J=21.7 Hz), 43.71, 51.45 (d, J=23.1 Hz), 92.55 (d, J=174.1 Hz), 116.36 (q, J=291.1 Hz), 162.98 (q, J=35.4 Hz).

It will be clear that the invention is well adapted to attain the ends and advantages mentioned as well as those inherent therein. While presently preferred embodiments have been described for purposes of this disclosure, various changes and modifications may be made which are well within the scope of the invention. Numberous other changes may be made which will readily suggest themselves to those skilled in the art and which are encompassed in the spirit of the invention disclosed herein and as defined in the appended claims.

All references including publications and patents are incorporated by reference herein for all purposes.

1. A process for preparing a fluoroalkyl arylsulfinyl compound having a formula (I) as follows: 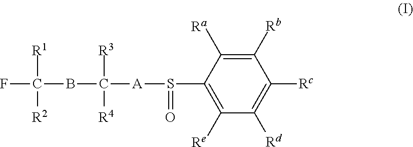
the process comprising reacting an oxygen-containing compound having a formula (II) with arylsulfur trifluoride having a formula (III) or with arylsulfur halotetrafluoride having a formula (IV), the reaction of the oxygen-containing compound of formula (II) and arylsulfur halotetrafluoride of formula (IV) being in the presence of a reducing substance that reduces the arylsulfur halotetrafluoride: 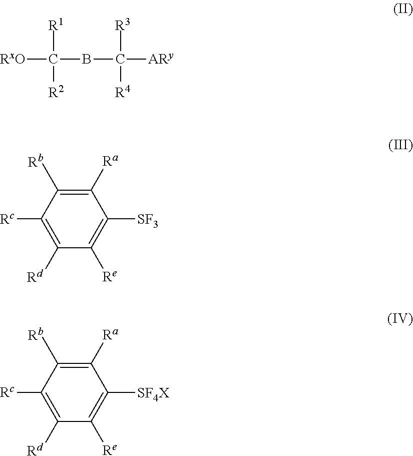
in which:
R¹, R², R³, and R⁴ each is independently a hydrogen atom, a substituted or unsubstituted alkyl group having 1 to 20 carbon atoms, a substituted or unsubstituted alkenyl group having 2 to 20 carbon atoms, a substituted or unsubstituted alkynyl group having 2 to 20 carbon atoms, a substituted or unsubstituted aryl group having 6 to 20 carbon atoms, a substituted or unsubstituted heterocyclyl group having 1 to 20 carbon atoms, a substituted or unsubstituted alkoxy group having 1 to 20 carbon atoms, a substituted or unsubstituted acyl group having 1 to 20 carbon atoms, a substituted or unsubstituted alkoxycarbonyl group having 2 to 20 carbon atoms, a substituted or unsubstituted aryloxycarbonyl group having 7 to 20 carbon atoms, a substituted or unsubstituted (heterocyclyl)oxycarbonyl group having 2 to 20 carbon atoms, or a cyano group;
R^{a}, R^{b}, R^{c}, R^{d}, and R^{e} each is independently a hydrogen atom, a halogen atom, a substituted or unsubstituted alkyl group having 1 to 10 carbon atoms, a nitro group, or a cyano group;
A is an oxygen atom or NR⁵ in which R⁵ is a hydrogen atom, a substituted or unsubstituted alkyl group having 1 to 20 carbon atoms, a substituted or unsubstituted aryl group having 6 to 20 carbon atoms, a substituted or unsubstituted heterocyclyl group having 1 to 20 carbon atoms, a substituted or unsubstituted acyl group having 1 to 20 carbon atoms, a substituted or unsubstituted alkoxycarbonyl group having 2 to 20 carbon atoms, a substituted or unsubstituted aryloxycarbonyl group having 7 to 20 carbon atoms, a substituted or unsubstituted (heterocyclyl)oxycarbonyl group having 2 to 20 carbon atoms, a substituted or unsubstituted alkylsulfonyl group having 1 to 20 carbon atoms, a substituted or unsubstituted arylsulfonyl group having 6 to 20 carbon atoms, a substituted or unsubstituted (heterocyclyl)sulfonyl group having 1 to 20 carbon atoms, or a R¹⁴R¹⁵R¹⁶Si group in which R¹⁴, R¹⁵, and R¹⁶ each is independently an alkyl group having 1 to 10 carbon atoms, an aralkyl group having 7 to 10 carbon atoms, or an aryl group having 6 to 10 carbon atoms;
B is: 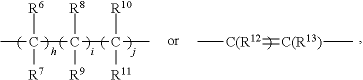
in which:
R⁶, R⁷, R⁸, R⁹, R¹⁰, R¹¹, R¹², and R¹³ each is independently a hydrogen atom, a halogen atom, a substituted or unsubstituted alkyl group having 1 to 20 carbon atoms, a substituted or unsubstituted alkenyl group having 2 to 20 carbon atoms, a substituted or unsubstituted alkynyl group having 2 to 20 carbon atoms, a substituted or unsubstituted aryl group having 6 to 20 carbon atoms, a substituted or unsubstituted heterocyclyl group having 1 to 20 carbon atoms, a substituted or unsubstituted alkoxy group having 1 to 20 carbon atoms, a substituted or unsubstituted aryloxy group having 6 to 20 carbon atoms, a substituted or unsubstituted (heterocyclyl)oxy group having 1 to 20 carbon atoms, a substituted or unsubstituted acyl group having 1 to 20 carbon atoms, a substituted or unsubstituted acyloxy group having 1 to 20 carbon atoms, a substituted or unsubstituted alkoxycarbonyl group having 2 to 20 carbon atoms, a substituted or unsubstituted aryloxycarbonyl group having 7 to 20 carbon atoms, a substituted or unsubstituted (heterocyclyl)oxycarbonyl group having 2 to 20 carbon atoms, a substituted or unsubstituted amino group having 20 or less carbon atoms, a substituted or unsubstituted carbamoyl group having 1 to 20 carbon atoms, a substituted or unsubstituted alkylthio group having 1 to 20 carbon atoms, a substituted or unsubstituted arylthio group having 6 to 20 carbon atoms, a substituted or unsubstituted (heterocyclyl)thio group having 1 to 20 carbon atoms, a substituted or unsubstituted alkylsulfinyl group having 1 to 20 carbon atoms, a substituted or unsubstituted arylsulfinyl group having 6 to 20 carbon atoms, a substituted or unsubstituted (heterocyclyl)sulfinyl group having 1 to 20 carbon atoms, a substituted or unsubstituted alkylsulfonyl group having 1 to 20 carbon atoms, a substituted or unsubstituted arylsulfonyl group having 6 to 20 carbon atoms, a substituted or unsubstituted (heterocyclyl)sulfonyl group having 1 to 20 carbon atoms, a nitro group, or a cyano group;
h, i, and j each is independently 0 or 1;
R^{x} and R^{y} each is independently a hydrogen atom, a silyl group, a metal atom, an ammonium moiety, or a phosphonium moiety, or R^{x} and R^{y} combine to form a metal atom or a silyl group;
X is a chlorine atom, bromine atom, or iodine atom; and
two or more of R¹, R², R³, R⁴, R⁵, R⁶, R⁷, R⁸, R⁹, R¹⁰, and R¹¹ or two or more of R¹, R², R³, R⁴, R⁵, R¹², and R¹³ may be linked with or without a heteroatom(s) to form any ring structure.

2. The process of claim 1 wherein at least one of R^{x} and R^{y} is a hydrogen atom or a silyl group, or R^{x} and R^{y} combine to form a silyl group, in which the reaction of the oxygen-containing compound of formula (II) with the arylsulfur trifluoride of formula (III) is conducted in the presence of a base or a silicon atom-activating agent.

3. The process of claim 1, wherein the arylsulfur trifluoride of formula (III) is first prepared by a process comprising:
reacting arylsulfur halotetrafluoride of formula (IV) with a reducing substance: 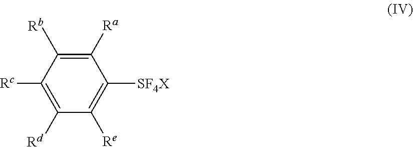
in which:
R^{a}, R^{b}, R^{c}, R^{d}, and R^{e} each is independently a hydrogen atom, a halogen atom, a substituted or unsubstituted alkyl group having 1 to 10 carbon atoms, a nitro group, or a cyano group; and
X is a chlorine atom, bromine atom, or iodine atom.

4. The process of claim 3, wherein X is a chlorine atom.

5. The process of claim 3, wherein the reducing substance is a substituted or unsubstituted heteroarene.

6. (canceled)

7. The process of claim 1, wherein X is a chlorine atom.

8. The process of claim 1, wherein the reducing substance is a substituted or unsubstituted heteroarene.

9.-10. (canceled)

11. A process for preparing a fluoroalkyl arylsulfinyl compound having a formula (Ia) as follows: 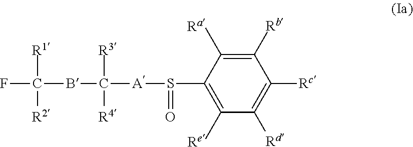
the process comprising reacting an oxygen-containing compound having a formula (IIa) with arylsulfur trifluoride having a formula (IIIa) or with arylsulfur halotetrafluoride having a formula (IVa), the reaction of the oxygen-containing compound of formula (IIa) and arylsulfur halotetrafluoride of formula (IVa) being in the presence of a reducing substance that reduces the arylsulfur halotetrafluoride: 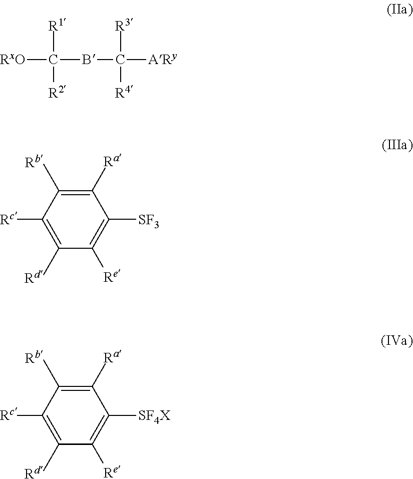
in which:
R^{1′}, R^{2′}, R^{3′}, and R^{4′} each is independently a hydrogen atom, a substituted or unsubstituted alkyl group having 1 to 20 carbon atoms, a substituted or unsubstituted alkenyl group having 2 to 20 carbon atoms, a substituted or unsubstituted aryl group having 6 to 20 carbon atoms, a substituted or unsubstituted alkoxycarbonyl group having 2 to 20 carbon atoms, a substituted or unsubstituted aryloxycarbonyl group having 7 to 20 carbon atoms, or a cyano group;
R^{a′}, R^{b′}, R^{c′}, R^{d′}, and R^{e′} each is independently a hydrogen atom, a halogen atom, a substituted or unsubstituted alkyl group having 1 to 10 carbon atoms, or a nitro group;
A′ is an oxygen atom or NR^{5′} in which R^{5′} is a hydrogen atom, a substituted or unsubstituted alkyl group having 1 to 20 carbon atoms, or a substituted or unsubstituted aryl group having 6 to 20 carbon atoms;
B′ is 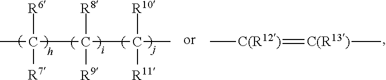
in which:
R^{6′}, R^{7′}, R^{8′}, R^{9′}, R^{10′}, R^{11′}, R^{12′} and R^{13′} each is independently a hydrogen atom, a halogen atom, a substituted or unsubstituted alkyl group having 1 to 20 carbon atoms, a substituted or unsubstituted alkenyl group having 2 to 20 carbon atoms, a substituted or unsubstituted aryl group having 6 to 20 carbon atoms, a substituted or unsubstituted alkoxy group having 1 to 20 carbon atoms, a substituted or unsubstituted aryloxy group having 6 to 20 carbon atoms, a substituted or unsubstituted alkoxycarbonyl group having 2 to 20 carbon atoms, a substituted or unsubstituted aryloxycarbonyl group having 7 to 20 carbon atoms, a nitro group, or a cyano group;
h, i, and j each is independently 0 or 1;
R^{x} and R^{y} each is independently a hydrogen atom, a silyl group, a metal atom, an ammonium moiety, or a phosphonium moiety; or R^{x} and R^{y} combine to form a metal atom or a silyl group;
X is a chlorine atom, bromine atom, or iodine atom; and
two or more of R^{1′}, R^{2′}, R^{3′}, R^{4′}, R^{5′}, R^{6′}, R^{7′}, R^{8′}, R^{9′}, R^{10′} and R^{11′} or two or more of R^{1′}, R^{2′}, R^{3′}, R^{4′}, R^{5′}, R^{12′}, and R^{13′} may be linked with or without a heteroatom(s) to form any ring structure.

12. The process of claim 11, wherein the arylsulfur trifluoride of formula (IIIa) is first prepared by a process comprising reacting arylsulfur halotetrafluoride of formula (IVa) with a reducing substance: 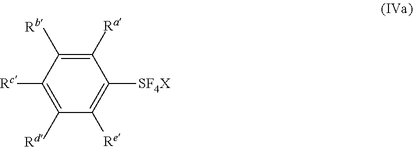
in which:
R^{a′}, R^{b′}, R^{c′}, R^{d′}, and R^{e′} each is independently a hydrogen atom, a halogen atom, a substituted or unsubstituted alkyl group having 1 to 10 carbon atoms, or a nitro group; and
X is a chlorine atom, bromine atom, or iodine atom.

13. The process of claim 12, wherein X is a chlorine atom.

14. The process of claim 12, wherein the reducing substance is a substituted or unsubstituted heteroarene.

15. (canceled)

16. The process of claim 11, wherein X is a chlorine atom.

17. The process of claim 11, wherein the reducing substance is a substituted or unsubstituted heteroarene.

18.-20. (canceled)

21. A process for preparing a fluoroalkyl arylsulfinyl compound having a formula (Ie) as follows: 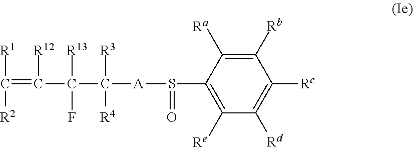
the process comprising reacting an oxygen-containing compound having a formula (IIb) with arylsulfur trifluoride having a formula (III) or with arylsulfur halotetrafluoride having a formula (IV), the reaction of the oxygen-containing compound of formula (IIb) and arylsulfur halotetrafluoride of formula (IV) being in the presence of a reducing substance that reduces the arylsulfur halotetrafluoride: 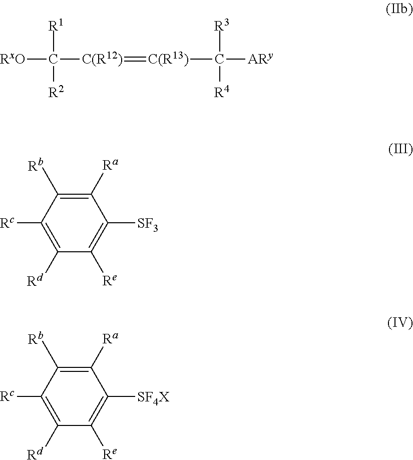
in which:
R¹, R², R³, and R⁴ each is independently a hydrogen atom, a substituted or unsubstituted alkyl group having 1 to 20 carbon atoms, a substituted or unsubstituted alkenyl group having 2 to 20 carbon atoms, a substituted or unsubstituted alkynyl group having 2 to 20 carbon atoms, a substituted or unsubstituted aryl group having 6 to 20 carbon atoms, a substituted or unsubstituted heterocyclyl group having 1 to 20 carbon atoms, a substituted or unsubstituted alkoxy group having 1 to 20 carbon atoms, a substituted or unsubstituted acyl group having 1 to 20 carbon atoms, a substituted or unsubstituted alkoxycarbonyl group having 2 to 20 carbon atoms, a substituted or unsubstituted aryloxycarbonyl group having 7 to 20 carbon atoms, a substituted or unsubstituted (heterocyclyl)oxycarbonyl group having 2 to 20 carbon atoms, or a cyano group;
R^{a}, R^{b}, R^{c}, R^{d}, and R^{e} each is independently a hydrogen atom, a halogen atom, a substituted or unsubstituted alkyl group having 1 to 10 carbon atoms, a nitro group, or a cyano group;
A is an oxygen atom or NR⁵ in which R⁵ is a hydrogen atom, a substituted or unsubstituted alkyl group having 1 to 20 carbon atoms, a substituted or unsubstituted aryl group having 6 to 20 carbon atoms, a substituted or unsubstituted heterocyclyl group having 1 to 20 carbon atoms, a substituted or unsubstituted acyl group having 1 to 20 carbon atoms, a substituted or unsubstituted alkoxycarbonyl group having 2 to 20 carbon atoms, a substituted or unsubstituted aryloxycarbonyl group having 7 to 20 carbon atoms, a substituted or unsubstituted (heterocyclyl)oxycarbonyl group having 2 to 20 carbon atoms, a substituted or unsubstituted alkylsulfonyl group having 1 to 20 carbon atoms, a substituted or unsubstituted arylsulfonyl group having 6 to 20 carbon atoms, a substituted or unsubstituted (heterocyclyl)sulfonyl group having 1 to 20 carbon atoms, or a R¹⁴R¹⁵R¹⁶Si group in which R¹⁴, R¹⁵, and R¹⁶ each is independently an alkyl group having 1 to 10 carbon atoms, an aralkyl group having 7 to 10 carbon atoms, or an aryl group having 6 to 10 carbon atoms;
R¹² and R¹³ each is independently a hydrogen atom, a halogen atom, a substituted or unsubstituted alkyl group having 1 to 20 carbon atoms, a substituted or unsubstituted alkenyl group having 2 to 20 carbon atoms, a substituted or unsubstituted alkynyl group having 2 to 20 carbon atoms, a substituted or unsubstituted aryl group having 6 to 20 carbon atoms, a substituted or unsubstituted heterocyclyl group having 1 to 20 carbon atoms, a substituted or unsubstituted alkoxy group having 1 to 20 carbon atoms, a substituted or unsubstituted aryloxy group having 6 to 20 carbon atoms, a substituted or unsubstituted (heterocyclyl)oxy group having 1 to 20 carbon atoms, a substituted or unsubstituted acyl group having 1 to 20 carbon atoms, a substituted or unsubstituted acyloxy group having 1 to 20 carbon atoms, a substituted or unsubstituted alkoxycarbonyl group having 2 to 20 carbon atoms, a substituted or unsubstituted aryloxycarbonyl group having 7 to 20 carbon atoms, a substituted or unsubstituted (heterocyclyl)oxycarbonyl group having 2 to 20 carbon atoms, a substituted or unsubstituted amino group having 20 or less carbon atoms, a substituted or unsubstituted carbamoyl group having 1 to 20 carbon atoms, a substituted or unsubstituted alkylthio group having 1 to 20 carbon atoms, a substituted or unsubstituted arylthio group having 6 to 20 carbon atoms, a substituted or unsubstituted (heterocyclyl)thio group having 1 to 20 carbon atoms, a substituted or unsubstituted alkylsulfinyl group having 1 to 20 carbon atoms, a substituted or unsubstituted arylsulfinyl group having 6 to 20 carbon atoms, a substituted or unsubstituted (heterocyclyl)sulfinyl group having 1 to 20 carbon atoms, a substituted or unsubstituted alkylsulfonyl group having 1 to 20 carbon atoms, a substituted or unsubstituted arylsulfonyl group having 6 to 20 carbon atoms, a substituted or unsubstituted (heterocyclyl)sulfonyl group having 1 to 20 carbon atoms, a nitro group, or a cyano group;
R^{x} and R^{y} each is independently a hydrogen atom, a silyl group, a metal atom, an ammonium moiety, or a phosphonium moiety, or R^{x} and R^{y} combine to form a metal atom or a silyl group;
X is a chlorine atom, bromine atom, or iodine atom; and
two or more of R¹, R², R³, R⁴, R⁵, R¹², and R¹³ may be linked with or without a heteroatom(s) to form any ring structure.

22. The process of claim 21, wherein the arylsulfur trifluoride of formula (III) is prepared by the process comprising reacting arylsulfur halotetrafluoride of formula (IV) with a reducing substance: 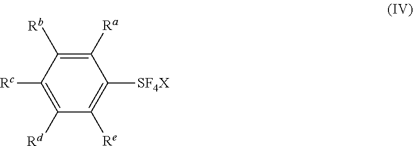
in which:
R^{a}, R^{b}, R^{c}, R^{d}, and R^{e} each is independently a hydrogen atom, a halogen atom, a substituted or unsubstituted alkyl group having 1 to 10 carbon atoms, a nitro group, or a cyano group; and
X is a chlorine atom, bromine atom, or iodine atom.

23. (canceled)

24. A process for preparing a fluoroalkyl arylsulfinyl compound having a formula (If) as follows: 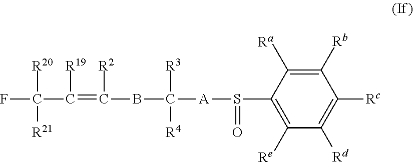
the process comprising reacting an oxygen-containing compound having a formula (IIc) with arylsulfur trifluoride having a formula (III) or with arylsulfur halotetrafluoride having a formula (IV), the reaction of the oxygen-containing compound of formula (IIc) and arylsulfur halotetrafluoride of formula (IV) being in the presence of a reducing substance that reduces the arylsulfur halotetrafluoride: 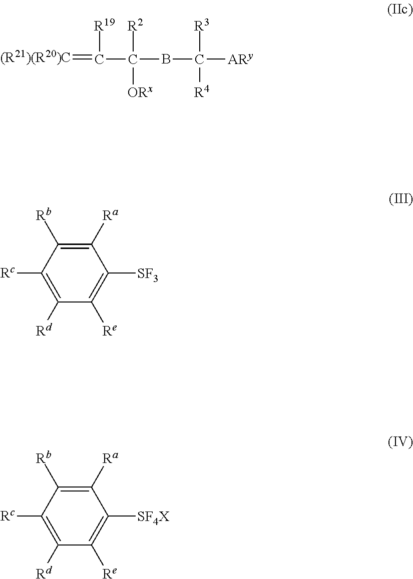
in which:
R², R³, and R⁴ each is independently a hydrogen atom, a substituted or unsubstituted alkyl group having 1 to 20 carbon atoms, a substituted or unsubstituted alkenyl group having 2 to 20 carbon atoms, a substituted or unsubstituted alkynyl group having 2 to 20 carbon atoms, a substituted or unsubstituted aryl group having 6 to 20 carbon atoms, a substituted or unsubstituted heterocyclyl group having 1 to 20 carbon atoms, a substituted or unsubstituted alkoxy group having 1 to 20 carbon atoms, a substituted or unsubstituted acyl group having 1 to 20 carbon atoms, a substituted or unsubstituted alkoxycarbonyl group having 2 to 20 carbon atoms, a substituted or unsubstituted aryloxycarbonyl group having 7 to 20 carbon atoms, a substituted or unsubstituted (heterocyclyl)oxycarbonyl group having 2 to 20 carbon atoms, or a cyano group;
R^{a}, R^{b}, R^{c}, R^{d}, and R^{e} each is independently a hydrogen atom, a halogen atom, a substituted or unsubstituted alkyl group having 1 to 10 carbon atoms, a nitro group, or a cyano group;
A is an oxygen atom or NR⁵ in which R⁵ is a hydrogen atom, a substituted or unsubstituted alkyl group having 1 to 20 carbon atoms, a substituted or unsubstituted aryl group having 6 to 20 carbon atoms, a substituted or unsubstituted heterocyclyl group having 1 to 20 carbon atoms, a substituted or unsubstituted acyl group having 1 to 20 carbon atoms, a substituted or unsubstituted alkoxycarbonyl group having 2 to 20 carbon atoms, a substituted or unsubstituted aryloxycarbonyl group having 7 to 20 carbon atoms, a substituted or unsubstituted (heterocyclyl)oxycarbonyl group having 2 to 20 carbon atoms, a substituted or unsubstituted alkylsulfonyl group having 1 to 20 carbon atoms, a substituted or unsubstituted arylsulfonyl group having 6 to 20 carbon atoms, a substituted or unsubstituted (heterocyclyl)sulfonyl group having 1 to 20 carbon atoms, or a R¹⁴R¹⁵R¹⁶Si group in which R¹⁴, R¹⁵, and R¹⁶ each is independently an alkyl group having 1 to 10 carbon atoms, an aralkyl group having 7 to 10 carbon atoms, or an aryl group having 6 to 10 carbon atoms;
B is: 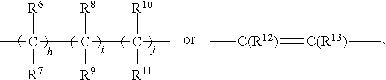
in which:
R⁶, R⁷, R⁸, R⁹, R¹⁰, R¹¹, R¹², R¹³, R¹⁹, R²⁰ and R²¹ each is independently a hydrogen atom, a halogen atom, a substituted or unsubstituted alkyl group having 1 to 20 carbon atoms, a substituted or unsubstituted alkenyl group having 2 to 20 carbon atoms, a substituted or unsubstituted alkynyl group having 2 to 20 carbon atoms, a substituted or unsubstituted aryl group having 6 to 20 carbon atoms, a substituted or unsubstituted heterocyclyl group having 1 to 20 carbon atoms, a substituted or unsubstituted alkoxy group having 1 to 20 carbon atoms, a substituted or unsubstituted aryloxy group having 6 to 20 carbon atoms, a substituted or unsubstituted (heterocyclyl)oxy group having 1 to 20 carbon atoms, a substituted or unsubstituted acyl group having 1 to 20 carbon atoms, a substituted or unsubstituted acyloxy group having 1 to 20 carbon atoms, a substituted or unsubstituted alkoxycarbonyl group having 2 to 20 carbon atoms, a substituted or unsubstituted aryloxycarbonyl group having 7 to 20 carbon atoms, a substituted or unsubstituted (heterocyclyl)oxycarbonyl group having 2 to 20 carbon atoms, a substituted or unsubstituted amino group having 20 or less carbon atoms, a substituted or unsubstituted carbamoyl group having 1 to 20 carbon atoms, a substituted or unsubstituted alkylthio group having 1 to 20 carbon atoms, a substituted or unsubstituted arylthio group having 6 to 20 carbon atoms, a substituted or unsubstituted (heterocyclyl)thio group having 1 to 20 carbon atoms, a substituted or unsubstituted alkylsulfinyl group having 1 to 20 carbon atoms, a substituted or unsubstituted arylsulfinyl group having 6 to 20 carbon atoms, a substituted or unsubstituted (heterocyclyl)sulfinyl group having 1 to 20 carbon atoms, a substituted or unsubstituted alkylsulfonyl group having 1 to 20 carbon atoms, a substituted or unsubstituted arylsulfonyl group having 6 to 20 carbon atoms, a substituted or unsubstituted (heterocyclyl)sulfonyl group having 1 to 20 carbon atoms, a nitro group, or a cyano group;
h, i, and j each is independently 0 or 1;
R^{x} and R^{y} each is independently a hydrogen atom, a silyl group, a metal atom, an ammonium moiety, or a phosphonium moiety, or R^{x} and R^{y} combine to form a metal atom or a silyl group;
X is a chlorine atom, bromine atom, or iodine atom; and
two or more of R², R³, R⁴, R⁵, R⁶, R⁷, R⁸, R⁹, R¹⁰, R¹¹, R¹², R¹³, R¹⁹, R²⁰ and R²¹ or two or more of R², R³, R⁴, R⁵, R¹², R¹³, R¹⁹, R²⁰ and R²¹ may be linked with or without a heteroatom(s) to form any ring structure.

25. The process of claim 24, wherein the arylsulfur trifluoride of formula (III) is first prepared by the process comprising reacting arylsulfur halotetrafluoride of formula (IV) with a reducing substance: 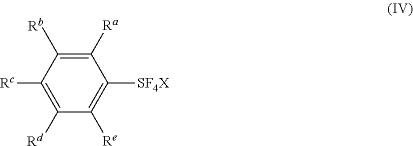
in which:
R^{a}, R^{b}, R^{c}, R^{d}, and R^{e} each is independently a hydrogen atom, a halogen atom, a substituted or unsubstituted alkyl group having 1 to 10 carbon atoms, a nitro group, or a cyano group; and
X is a chlorine atom, bromine atom, or iodine atom.

26. (canceled)

27. The process of claim 11 wherein at least one of R^{x} and R^{y} is a hydrogen atom or a silyl group, or IV and R^{y} combine to form a silyl group, in which the reaction of the oxygen-containing compound of formula (IIa) with the arylsulfur trifluoride of formula (IIIa) is conducted in the presence of a base or a silicon atom-activating agent.

28. The process of claim 21 wherein at least one of R^{x} and R^{y} is a hydrogen atom or a silyl group, or R^{x} and R^{y} combine to form a silyl group, in which the reaction of the oxygen-containing compound of formula (IIb) with the arylsulfur trifluoride of formula (III) is conducted in the presence of a base or a silicon atom-activating agent.

29. The process of claim 24 wherein at least one of R^{x} and R^{y} is a hydrogen atom or a silyl group, or R^{x} and R^{y} combine to form a silyl group, in which the reaction of the oxygen-containing compound of formula (IIc) with the arylsulfur trifluoride of formula (III) is conducted in the presence of a base or a silicon atom-activating agent.
